(19) United States
(12) Patent Application Publication
(10) Pub. No.: US 2005/0009820 A1
(43) Pub. Date: Jan. 13, 2005

(54) Thiochromenones used to combat painful conditions and neurodegenerative diseases

(76) Inventors: Heinrich Meier, Leverkusen (DE); Swen Allerheilingen, Essen (DE); Michael Gerisch, Wuppertal (DE); Rudolfd Schohe-Loop, Wuppertal (DE); Arnd Voerste, Koln (DE); Frank Mauler, Overath (DE); Jean Marie Victor de Vry, Rosrath (DE); Thomas Muller, Wuppertal (DE); Christoph Methfessel, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/479,189

(22) PCT Filed: May 21, 2002
(86) PCT No.: PCT/EP02/05538

(30) Foreign Application Priority Data
May 31, 2001 (DE) 101 26 434.8

(51) Int. Cl. A61K 31/5377; A61K 31/382; C07D 495/02

(52) U.S. Cl. 514/232.2; 514/432; 549/23; 544/145

(57) ABSTRACT

The invention relates to novel thiochromenones and processes for their preparation, to their use for the treatment and/or prophylaxis of diseases, especially for the treatment and/or prophylaxis of states of pain and neurodegenerative disorders.

The invention relates to novel thiochromenones and processes for their preparation, to their use for the treatment and/or prophylaxis of diseases, especially for the treatment and/or prophylaxis of states of pain and neurodegenerative disorders.

The amino acid L-glutamate is the principal excitatory neurotransmitter in the brain. Glutamate receptors can be divided into two large classes: 1. ionotropic receptors which control ion channels directly, and 2. metabotropic receptors (mGluRs).

Metabotropic glutamate receptors are a heterogeneous class of G protein-coupled receptors which, activated by glutamate, are able to activate various second messenger cascades. The second messenger cascades culminate in the modulation of numerous intracellular processes, including regulation of presynaptic glutamate release and regulation of postsynaptic ionotropic glutamate receptors.

At present, 8 different. subtypes of metabotropic glutamate receptors differing in second messenger cascade, pharmacology and localization in the brain are known (review: Ann. Rev. Pharnacol. Toxicol. 1997, 37, 205).

U.S. Pat. No. 4,221,800 and U.S. Pat. No. 4,571,405 describe the antiallergic effect of thioxanthen-9-ones.

The preparation and the antischistosomal effect of thioxanthen-9-ones and 2,3-cyclopentathiochromones are disclosed in U.S. Pat. No. 3,312,598, GB 803,803, GB 804,689, GB 805,870, Chem. Abstr. 54, 7740d (DE 1024981), Chem. Abstr. 62, 11763h and J. Med. Chem. 1967, 10, 867-876.

The synthesis of 2-chloro-6,7,8,9,10,10a-hexahydrocyclohepta[b]thiochromen-11(5aH)-one is described in Liebigs Ann. 1964,680,40-51.

2,3-Cyclopentathiochromones are disclosed as analgesics and antiinflanmatory agents in CAPLUS 1984, 610989 (JP 59112983).

The present invention relates to compounds of the general formula (I) 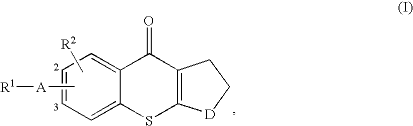
in which
the radical R¹-A- is located at either of positions 2 or 3 of the thiochromenone ring,
R¹ is (C₆-C₁₀)-aryl or 5- to 10-membered heteroaryl, where aryl and heteroaryl are optionally substituted identically or differently by radicals selected from the group of halogen, formyl, carbamoyl, cyano, hydroxyl, trifluoromethoxy, nitro, —NR³R⁴, tetrazolyl, (C₁-C₆)-alkoxycarbonyl and optionally hydroxyl-, morpholinyl-, (C₁-C₆)-acyloxy- or halogen-substituted (C₁-C₆)-alkyl, (C₁-C₆)-alkoxy, (C₁-C₆)-acyl and (C₁-C₆)-alkylthio,
   in which
   R³ and R⁴ are, independently of one another, hydrogen, (C₁-C₆)-alkyl or (C₁-C₆)-acyl,
      is 3- to 12-membered carbocyclyl or 4- to 12-membered heterocyclyl, where carbocyclyl and heterocyclyl are optionally substituted identically or differently by radicals selected from the group of (C₁-C₆)-alkyl, (C₁-C₆)-alkoxy, (C₁-C₆)-acyl, (C₁-C₆)-alkoxycarbonyl or oxo,
   or
   is a group of the formula R⁵-E-,
   in which
   E is optionally unsaturated (C₁-C₁₀)-alkanediyl, and
   R⁵ is hydrogen, carbamoyl, halogen, hydroxyl, nitro, trifluoromethyl, amino, mono-(C₁-C₆)-alkylamino, di-(C₁-C₆)-alkylamino, (C₁-C₆)-alkoxy, (C₆-C₁₀)-aryl, 5- to 10-membered heteroaryl or 4- to 10-membered, optionally oxo- and/or (C₁-C₆)-alkyl-substituted, optionally benzo-fused heterocyclyl, where aryl, heteroaryl and benzo in turn may be substituted by radicals selected from the group of halogen, cyano, trifluoromethyl, trifluoromethoxy, nitro and (C₁-C₆)-alkyl,
A is a bond or a group of the formula O, S, NR⁶, CO, SO, SO₂, SO₂—O, CO—NR⁷, SO₂—NR⁸, O—SO₂, NR⁹—CO, NR¹⁰—SO₂, NR¹¹—SO₂—O, NR¹²—SO₂—NR¹³ or NR⁴—CO—NR¹⁵,
   in which
   R⁶, R⁷, R⁸, R⁹, R¹⁰, R¹¹, R¹², R¹³, R¹⁴ and R¹⁵ are (C₃-C₈)-cycloalkyl or optionally unsaturated (C₁-C₆)-alkyl which is optionally substituted by hydroxyl, phenyl, (C₁-C₆)-alkoxy, (C₁-C₆)-alkoxycarbonyl or (C₃-C₈)-cycloalkyl, where phenyl in turn may be substituted by halogen or (C₁-C₄)-alkyl, or
in which
   R⁶, R⁷, R⁹, R¹⁰, R¹¹, R¹², R¹⁴and R⁵ are hydrogen,
or
the radical R¹-A- is hydrogen or ammo,
R² is hydrogen, halogen or (C₁-C₆)-alkyl or (C₁-C₆)-alkoxy, where alkyl and alkoxy are optionally substituted up to twice identically or differently by radicals selected from the group of hydroxyl, (C₁-C₆)-alkoxy, mono- and di-(C₁-C₆)-alkylamino, and
D is an optionally fluorine-substituted, divalent hydrocarbon radical having 3 to 10 carbon atoms,
and the salts, hydrates and/or solvates thereof,
with the exception of 2-chloro-6,7,8,9,10,10a-hexahydrocyclohepta[b]thiochromen-11(5aH)-one.

The compounds of the invention may exist in stereoisomeric forms which either are related as image and mirror image (enantiomers) or which are not related as image and mirror image (diastereomers). The invention relates both to the enantiomers or diastereomers or respective mixtures thereof. These mixtures of enantiomers and diastereomers can be separated in a known manner into the stereoisomerically pure constituents.

The compounds of the invention may also exist in the form of their salts, hydrates and/or solvates.

Salts which are preferred for the purposes of the invention are physiologically acceptable salts of the compounds of the invention.

Physiologically acceptable salts of the compounds of the invention may be acid addition salts of the compounds with mineral acids, carboxylic acids or sulfonic acids. Particularly preferred examples are salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, flimaric acid, maleic acid or benzoic acid.

Salts which may also be mentioned, however, are salts with conventional bases such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylairune, procaine, dibenzylamine, N-methylmorpholine, dihydroabietylarnine, 1-ephenamine or methylpiperidine.

Hydrates of the compounds of the invention are stoichiometric compositions of the compounds or its salts with water.

Solvates of the compounds of the invention are stoichiometric compositions of the compounds or its salts with solvent.

For the purposes of the present invention, the substituents generally have the following meaning:

(C₁-C₆)-Acyl is a straight-chain or branched acyl radical having 1 to 6 carbon atoms. Examples which may be mentioned are: acetyl, ethylcarbonyl, propylcarbonyl, isopropylcarbonyl, n-butylcarbonyl, pivaloyl, isobutylcarbonyl, pentylcarbonyl and hexylcarbonyl. A straight-chain or branched acyl radical having 1 to 4 carbon atoms is preferred. Acetyl and ethylcarbonyl are particularly preferred.

(C₁-C₁₀)-Alkanediyl is a straight-chain or branched alkanedjyl radical having 1 to 10 carbon atoms, it being possible for the two free valencies of the alkanediyl radical to be on one carbon atom (geminal), on adjacent carbon atoms (vicinal) or on nonadjacent carbon atoms. A straight-chain or branched alkanediyl radical having 3 to 8, particularly preferably having 3 to 6, carbon atoms is preferred. Examples which may be mentioned are methylene, ethylene, propylene, propane-1,2-diyl, propane-2,2-diyl, 2-methylpropane-1,3-diyl, butane-1,3-diyl, butane-2,4-diyl, pentane-2,4-diyl, 2-methylpentane-2,4-diyl.

(C₁-C₆)-Alkoxy is a straight-chain or branched alkoxy radical having 1 to 6 carbon atoms. A straight-chain or branched alkoxy radical having 1 to 4 carbon atoms is preferred. Examples which may be mentioned are: methoxy, ethoxy, n-propoxy, isopropoxy, tert-butoxy, n-pentoxy and n-hexoxy. A straight-chain or branched alkoxy radical having 1 to 3 carbon atoms is particularly preferred.

(C₁-C₆)-Alkoxycarbonyl is a straight-chain or branched alkoxycarbonyl radical having 1 to 6 carbon atoms. A straight-chain or branched alkoxycarbonyl radical having 1 to 4 carbon atoms is preferred. Examples which may be mentioned are: methoxycarbonyl, ethoxycarbonyl, n-propoxycarbonyl, isopropoxycarbonyl and tert-butoxycarbonyl. A straight-chain or branched alkoxycarbonyl radical having 1 to 3 carbon atoms is particularly preferred.

(C₁-C₆)- and (C₁-C₃)-alkyl is a straight-chain or branched alkyl radical having, respectively, 1 to 6 and 1 to 3 carbon atoms. A straight-chainor branched alkyl radical having 1 to 4, particularly preferably having 1 to 3, carbon atoms is preferred. Examples which may be mentioned are: methyl, ethyl, n-propyl, isopropyl, tert-butyl, n-pentyl and n-hexyl.

(C₁-C₆)-Alkylamino is a straight-chain or branched alkylamino radical having 1 to 6 carbon atoms. A straight-chain or branched alkylamino radical having 1 to 4 carbon atoms is preferred. Examples which may be mentioned are: methylamino, ethylamino, n-propylamino, isopropylamino, tert-butylamino, n-pentylamino and n-hexylamino. A straight-chain or branched alkylamino radical having 1 to 3 carbon atoms is particularly preferred.

(C₁-C₆)-Dialkylamino is a straight-chain or branched dialkylamino radical, where the alkyl radicals may be identical or different and each contain 1 to 6 carbon atoms. A straight-chain or branched dialkylamino radical is preferred, with the alkyl radical containing in each case 1 to 4 carbon atomns. Examples which may be mentioned are: dimethylamino, diethylamino, di-n-propylamino, diisopropylarino, di-t-butylamino, di-n-pentylamino, di-n-hexylamino, ethylmethylammno, isopropylmethylamino, n-butylethylamino, n-hexyl-i-pentylamino. A straight-chain or branched alkylamino radical having 1 to 3 carbon atoms is particularly preferred.

(C₁-C₆)-Alkylthio is a straight-chain or branched alkylthio radical having 1 to 6 carbon atoms. A straight-chain or branched alkylthio radical having 1 to 4 carbon atoms is preferred. Examples which may be mentioned are: methylthio, ethylthio, n-propylthio, isopropylthio, tert-butylthio, n-pentylthio and n-hexylthio. A straight-chain or branched alkylthio radical having 1 to 3 carbon atoms is particularly preferred.

(C₆-C₁₀)-Aryl is generally an aromatic radical having 6 to 10 carbon atoms. Preferred aryl radicals are phenyl and naphthyl.

3- to 12-membered carbocyclyl is a mono- or polycyclic, carbocyclic radical having 3 to 12 nirng atoms. 3- to 10-membered, in particular 3- to 8-membered, carbocyclyl are preferred. Mono- or bicyclic carbocyclyl is preferred. Monocyclic carbocyclyl is particularly preferred. The carbocyclyl radical may be saturated or partially unsaturated. Saturated carbocyclyi radicals are preferred. Likewise preferred are (C₃-C₁₀)-cycloalkyl, very particularly (C₄-C₇)-cycloalkyl. Examples which may be mentioned are: cyclopropyl, cyclobutyl, cyclopentyl, cyclopentenyl, cyclohexyl, cycloheptyl, norbom-1-yl, norbom-2-yl, norbom-7-yl, norbom-2-en-7-yl, cyclooctyl, cubyl, cyclononyl, cyclodecyl, decalinyl, adamant-1-yl, adamant-2-yl.

(C₃-C₈)-Cycloalkane-1,1-diyl is cyclopropane-1,1diyl, cyclobutane-1,1-diyl, cyclopentane-1,1-diyl or cyclohexane-1,1-diyl.

(C₃-C₈)-Cycloalkyl is cyclopropyl, cyclopentyl, cyclobutyl, cyclohexyl, cycloheptyl or cyclooctyl. The following may be mentioned as preferred: cyclopropyl, cyclopentyl and cyclohexyl.

Halogen is fluorine, chlorine, bromine and iodine. Fluorine, chlorine and bromine are preferred. Fluorine and chlorine are particularly preferred.

5- to 10-membered heteroaryl is an aromatic, mono- or bicyclic radical having 5 to 10 ring atoms and up to 5 heteroatoms from the series S, O and/or N, 5- to 6-membered heteroaryls having up to 4 heteroatoms are preferred. The heteroaryl radical may be bonded via a carbon atom or heteroatom. Examples which may be mentioned are: thienyl, furyl, pyrrolyl, thiazolyl, oxazolyl, imidazolyl, tetrazolyl, pyridyl, pyrimidyl, pyridazinyl, indolyl, indazolyl, benzofuranyl, benzothiophenyl, quinolinyl, isoquinolinyl.

4- to 12-membered or 4- to 10-membered heterocvclvi is a mono- or polycyclic, heterocyclic radical having, respectively, 4 to 12 or 10 ring atoms and up to 4, preferably up to 2, heteroatoms or hetero groups from the series N, O, S, SO, SO₂. 4- to 8-membered heterocyclyl is preferred. Mono- or bicyclic heterocyclyl is preferred. Monocyclic carbocyclyl is particularly preferred. N and O are preferred as heteroatoms. The heterocyclyl radicals may be saturated or partly unsaturated. Saturated heterocyclyl radicals are preferred. The heterocyclyl radicals may be bonded via a carbon atom or a heteroatom. 5- to 7-membered, monocyclic saturated heterocyclyl radicals having up to two heteroatoms from the series O, N and S are particularly preferred. Examples which may be mentioned are: oxetan-3-yl, pyrrolidin-2-yl, pyrrolidin-3-yl, pyrrolinyl, tetrahydrofuranyl, tetrahydrothienyl, pyranyl, piperidinyl, piperazinyl, thiopyranyl, morpholinyl, perhydroazepinyl, 1,5-dioxa-9-azaspiro[5,5]undecyl.

A divalent hydrocarbon radical having 3 to 10 carbon atoms is a straight-chain, branched, partly cyclic or cyclic, saturated or unsaturated organic radical which comprises 3 to 10 carbon atoms, which is linked via two bonds on one or two carbon atoms of the hydrocarbon radical to the adjacent atoms and which is saturated at the free valencies, depending on the degree of saturation and cyclization, with hydrogen atoms. Saturated organic radicals are preferred. Likewise preferred are hydrocarbon radicals having 3 to 8, particularly preferably having 3 to 6, carbon atoms. The hydrocarbon radical may consist of a straight-chain or branched alkanediyl radical, in which case two geminal, vicinal or nonadjacent hydrogen atoms of the alkanediyl radical may in turn be replaced by a straight-chain or branched alkanediyl radical. Examples which may be mentioned are: straight-chain or branched (C₃-C₁₀)-alkanediyl, (C₃-C₁₀)-cycloalkanediyl, and, with a total of 3 to 10 carbon atoms, mono- or dialkylcycloalkanediyl, cycloalkylalkanediyl, (yloalkyl)cycloalkyl, (ylocycloalkyl)alkyl and [(yloalkyl)cycloalkyl]alkyl. Examples which may be mentioned are: propylene, butylene, pentylene, butane-1,2-diyl, 2-ethylpropane-1,3-diyl, 2-methylethane-1,2-diyl, 2-methylpropane-1,2-diyl, 2-methylbutane-1,3-diyl, cyclobutane-1,1-diyl, cyclopentane-1,1-diyl, cyclohexane-1,1-diyl, cyclohexane-1,2-diyl, cyclohexane-1,3-diyl, 4,4-dimethylcyclohexane-1,1-diyl, 4-tert-butyl-cyclohexane-1,1-diyl, 2-cyclohexylpropane-1,3-diyl, 1-ylomethylcyclobutyl, 1-ylomethylcyclohexyl, 1-(2-yloethyl)cyclohexyl, 2-(2-yloethyl)cyclohexyl, [1-(ylomethyl)cyclobut-1-yl]methyl, [1-(ylomethyl)cyclohex-1-yl]methyl.

Oxo is a doubly bonded oxygen atom.

If radicals in the compounds of the invention are optionally substituted, the radicals may be substituted one or more times, identically or differently, unless specified otherwise. Substitution by up to three identical or different substituents is preferred.

If radicals in the. compounds of the invention are optionally unsaturated, the radical comprises, unless specified otherwise, one or more double or triple bonds which are optionally in conjugated or cumulative form. Double bonds are preferred. One double bond is particularly preferred.

One embodiment of the invention relates to compounds of the general formula (I),

in which the radical R¹-A- is located at position 3, the radical R² is located at position 2 of the thiochromenone ring, and R¹, A, R² and D have the meaning indicated above.

A further embodiment of the invention relates to compounds of the general formula (I),
in which
the radical R¹-A- is located at position 3 of the thiochromenone ring,
R¹ is (C₆-C₁₀)-aryl or 5- to 10-membered heteroaryl, where aryl and heteroaryl are optionally substituted identically or differently up to twice by radicals selected from the group of halogen, formyl, cyano, hydroxyl, hydroxymethyl, (C₁-C₆)-alkyl,
   is 4- to 10-membered heterocyclyl, where heterocyclyl are optionally substituted identically or differently by radicals selected from the group of (C₁-C₆)-alkyl, (C₁-C₆)-alkoxY, (C₁-C₆)-alkoxycarbonyl or oxo,
A is a bond or a group of the formula NR⁶, CO-Nk⁷, SO₂—NR⁸ or NR⁹—CO,
   in which
   R⁶, R⁷, R⁸ and R⁹ are hydrogen or optionally unsaturated (C₁-C₆)-alkyl which is optionally substituted up to twice, identically or differently, by hydroxyl or methoxy, or
   in which
   R⁶, R⁷ and R⁹ are hydrogen,
R² is hydrogen, and
D is a group of the formula (CH₂)ₘ—CR¹⁶R¹⁷—(CH₂)ₙ,
   in which the total number of carbon atoms is 3 to 10,
   m and n are identical or different and are a natural number from the series 0 to 6,
   and
   R¹⁶ and R¹⁷ are identical or different and are hydrogen or (C₁-C₆)-alkyl which is optionally substituted identically or differently by (C₃-C₅)-cycloalkyl or halogen,
   or
   CR¹⁶R¹⁷ is (C₃-C₆)-cycloalkane-1,1-diyl,
      and the salts, hydrates and/or solvates thereof.

A further embodiment of the invention relates to compounds of the general formula (I),
in which
the radical R¹-A- is located at position 3 of the thiochromenone ring,
R¹ is phenyl or 5- to 6-membered heteroaryl, where phenyl and heteroaryl are optionally substituted identically or differently up to twice by radicals selected from the group of halogen, cyano, (C₁-C₃)-alkyl,
   is 5- to 7-membered heterocyclyl, where heterocyclyl are optionally substituted identically or differently by radicals selected from the group of (C₁-C₃)-alkyl or oxo,
A is a bond or a group of the formula NR⁶, SO₂—NR⁸ or NR⁹—CO,
   in which
   R⁶, R⁸ and R⁹ are hydrogen or optionally unsaturated (C₁-C₃)-alkyl which is optionally substituted up to twice, identically or differently, by hydroxyl or methoxy, or
   in which
   R⁶ and R⁹ are hydrogen,
R² is hydrogen, and
D is a group of the formula (CH₂)ₘ—CR¹⁶R¹⁷—(CH₂)ₙ,
   in which
   the total number of carbon atoms is 3 to 6,
   m and n are identical or different and are a natural number from the series 0 to 2,
   and
   R¹⁶ and R¹⁷ are identical or different and are hydrogen or (C₁-C₃)-alkyl,
   or
   CR¹⁶R¹⁷ is (C₃-C₆)-cycloalkane-1,1-diyl,
      and the salts, hydrates and/or solvates thereof.

A further embodiment of the invention relates to compounds of the general formula (I),
in which
the radical R¹-A- is located at position 3 of the thiochromenone ring,
R¹, A and R² have the meaning indicated above, and
D is 2-methylpropane-1,2-diyl.

A further embodiment of the invention relates to compounds of the general formula (I),
in which
the radical R¹-A- is located at position 3 of the thiochromenone ring,
A is CO—NR⁷, SO₂—NR⁸ or NR⁹—CO,
   in which
   R⁷, R⁸ and R⁹ have the meaning indicated above,
R² is hydrogen, and
R¹ and D have the meaning indicated above.

The invention further relates to processes for preparing the compounds of the formula (I).

In process
[A] compounds of the general formula (II) 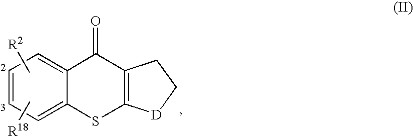
   in which
   R¹⁸ is located at one of positions 2 or 3 of the thiochromenone ring,
   R² and D have the meaning indicated above, and
   R¹⁸ is bromine or chlorine,
   are reacted with compounds of the general formula (III)
      R¹⁹—BR²⁰R²¹ (III),
   in which
   R¹⁹ is (C₆-C₁₀)-aryl or 5- to 10-membered heteroaryl, where aryl and heteroaryl are optionally substituted identically or differently by radicals selected from the group of halogen, formyl, carbamoyl, cyano, hydroxyl, trifluoromethoxy, nitro, —NR³R⁴, (C₁-C₆)-alkoxycarbonyl and optionally hydroxyl-, morpholinyl-, (C₁-C₆)-acyloxy- or halogen-substituted (C₁-C₆)-alkyl, (C₁-C₆)-alkoxy, (C₁-C₆)-acyl and (C₁-C₆)-alkylthio,
   in which
   R³ and R⁴ are, independently of one another, hydrogen, (C₁-C₆)-alkyl or (C₁-C₆)-acyl, and
   R²⁰ and R²¹ are hydroxyl,
   or
   BR²⁰R²¹ is 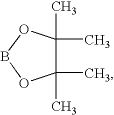
   to give compounds of the general formula (Ia) 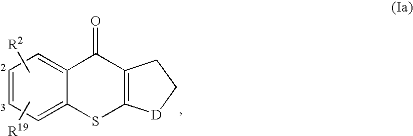
   in which
   R¹⁹ is located at one of positions 2 or 3 of the thiochromenone ring, and
   R², R¹⁹ and D have the meaning indicated above,
   in inert solvents under usual reaction conditions in the presence of a catalyst (see, for example: J. Tsuji, Palladium Reagents and Catalysts, J. Wiley & Sons, 1995; Suzuki coupling, review: N. Miyaura, A. Suzuki, Chem. Rev. 1995, 95, 2457-2483; H. Gröger, J. Prakt. Chem. 2000, 342, 334-339).

Process [A] is preferably carried out under Suzuki reaction conditions with palladium catalysts usual therefor, examples of particularly preferred catalysts being dichlorobis(triphenylphosphine)palladium, tetrakistriphenylphosphinepalladium(0), palladium(II) acetate or bis(diphenylphosphaneferrocenyl)palladium(II) chloride.

Suzuki reactions are carried out with usual additional reagents such as potassium acetate, cesium, potassium or sodium carbonate, barium hydroxide, potassium tert-butoxide, cesium fluoride or potassium phosphate, examples of particularly preferred additional reagents being potassium acetate and/or aqueous sodium carbonate solution.

Suzuki reactions are carried out in inert solvents which are not changed under the reaction conditions, and these include ethers such as dioxane, tetrahydrofuran or 1,2-dimethoxyethane, hydrocarbons such as benzene, xylene or toluene, or other solvents such as nitrobenzene, dimethylformamide, dimethylacetamide, dimethyl sulfoxide or N-methylpyrrolidone, examples of particularly preferred solvents being dimethylformamide, dimethylacetamide, dimethyl sulfoxide or 1,2-dimethoxyethane.

Process [A] is preferably carried out in a temperature range from room temperature to 130° C. under atmospheric pressure.

The compounds (III) are commnerically available or can be prepared by known methods or can be prepared for the reaction in situ as described below.

The biaryl syntheses which take place via boronates prepared in situ are prepared under usual reaction conditions in the presence of a catalyst, preferably in the presence of a transition metal catalyst, in particular in the presence of a palladium catalyst (see, for example, A. Giroux, Y. Han, P. Prasit, Tetrahedr. Lett. 1997, 38, 3841-44; T. Ishiyama, M. Murata, N. Miyaura, J. Org. Chem. 1995, 60, 7508-10.), preferably in dimethylformamide or dimethyl sulfoxide as solvent. The transition metal catalysts preferably used are palladiurn(0) or palladium(II) compounds, in particular bis(diphenylphosphaneferrocenyl)palladium(II) chloride. The reaction takes place in particular at a temperature from 70° C. to 110° C. in the presence of bases, preferably potassium acetate and/or aqueous sodium carbonate solution.

Also used besides Suzuki reactions are aryl coupling reaction with organotin (Stille coupling) or substituted olefins (Heck reaktion) under the reaction conditions usual therefor in the presence of a catalyst, preferably in the presence of a transition metal catalyst, in particular in the presence of a palladium catalyst (see, for example, J. Tsuji, Palladium Reagents and Catalysts, J. Wiley & Sons, 1995) preferably in dimethylformamide, N-methylpyrrolidone, 1,2-dimethoxyethane or toluene as solvent at a temperature of 60-140° C. The transition metal catalysts preferably used are palladium(0) or palladium(II) compounds, in particular bis(triphenylphos-phane)palladium(II) chloride, palladium(II) acetate or tetrakis(triphenylphosphane)-palladium(0). When organotin compounds are used (Stille coupling, review: V. Farina, V. Krishnamurthy, W. J. Scott in: The Stille Reaction, J. Wiley and Sons, New York; 1998), the reaction takes place in particular at a temperature from 110° C. to 130° C. When olefins are used (Heck reaction, review: I. P. Beletskaya, A. V. Cheprakov: The Heck Reaction as a Sharpening Stone of Palladium Catalysis, Chem. Rev. 2000, 100, 3009-3066.) the reaction takes place in particular at a temperature of 80-100° C. in the presence of a base, preferably triethylamine or aqueous sodium bicarbonate solution, and in the presence of a tetraalkylammonium or phosphonium salt, preferably tetrabutylammonium chloride or bromide.

The compounds of the general formula (II) can be prepared from the appropriate precursors for example in analogy to process [M].

The compounds of the general formula (III) are known or can be prepared by known processes.

In process
[B] compounds of the general formula (II) are reacted with compounds of the general formula (IV)
   R²²—H (IV),
   in which
   R²² is a 4- to 12-membered heterocyclyl which is bonded via N, where heterocyclyl comprises at least one nitrogen atom having a free valency, and is optionally substituted identically or differently by radicals selected from the group of (C₁-C₆)-alkyl, (C₁-C₆)-alkoxy, (C₁-C₆)-acyl, (C₁-C₆)-alkoxycarbonyl or oxo,
   to give compounds of the general formula (Ib) 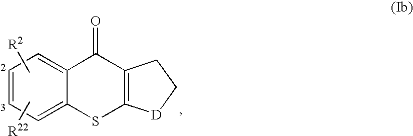
   in which
   R²² is located at one of positions 2 or 3 of the thiochromenone ring, and
   R¹, R²² and D have the meaning indicated above,
   in inert solvents under the conditions usual for such reactions.

In process
[C] compounds of the general formula (II) are reacted with compounds of the general formula (V)
   R¹-G-H (V),
   in which
   R¹ has the meaning indicated above, and
   G is O, S or NR⁶, in which R⁶ has the meaning indicated above,
   to give compounds of the general formula (Ic) 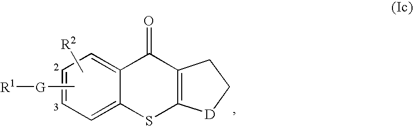
   in which
   R¹-G- is located at one of positions 2 or 3 of the thiochromenone ring, and
   R¹, R², D and G have the meaning indicated above,
   in inert solvents under usual reaction conditions in the presence of a catalyst.

Processes [B] and [C] are preferably carried out with catalysis by palladium (S. L. Buchwald, et al., J. Am. Chem. Soc. 1999, 121, 4369-4378; J. F. Hartwig, et al., J. Org. Chem. 1999, 64, 5575-5580; Buchwald aminations: J. P. Wolfe, S. L. Buchwald, J. Org. Chem. 2000, 65, 1144-1157; J. P. Wolfe, H. Tomori, J. P. Sadighi, J. Yin, S. L. Buchwald, J. Org. Chem. 2000, 65, 1158-1174) or Cu(I) (J. Lindley, Tetrahedron 1984, 40, 1433) examples of particularly preferred catalysts being tris(dibenzylideneacetone)dipalladium(0), palladium acetate or copper(I) iodide.

Processes [B] and [C] are optionally carried out with addition of bases such as, for example, alkali metal carbonates such as cesium carbonate, sodium or potassium carbonate, or sodium or potassium methanolate, or sodium or potassium methanolate, or sodium or potassium tert-butoxide, or other bases such as potassium phosphate, DBU, pyridine, triethylamine or diisopropylethylamine, examples of particularly preferred bases being potassium phosphate, sodium tert-butoxide, potassium carbonate, cesium carbonate, triethylamine, diisopropylethylamine or pyridine.

If the reaction is carried out with Pd catalysis, processes [B] and [C] are carried out where appropriate with addition of ligands, examples of particularly preferred ligands being 2-(di-t-butylphosphino)biphenyl, triphenylphosphine, [5-(diphenylphosphino)-9,9-dimethyl-9H-xanthen-4-yl](diphenyl)phosphine, 1,1′-biphenyl-2-yl(dicyclohexyl)phosphine or (+/−)-2,2-bis(diphenylphosphino)-1,1-binaphthyl.

Processes [B] and [C] are carried out in inert solvents which are not changed under the reaction conditions, and these include ethers such as dioxane, tetrahydrofuran or 1,2-dimnethoxyethane, hydrocarbons such as benzene, xylene or toluene, or other solvents such as dimethylformarnide, dimethylacetarnide, pyridine or N-methylpyrrolidone, examples of particularly preferred solvents being toluene, xylene, dimethylformaride, dimethylacetaride or pyridine.

Processes [B] and [C] are preferably carried out in a temperature range from room temperature up to refluxing of the solvents under atmospheric pressure.

The compounds of the general formulae (IV) and (V) are known or can be prepared by known processes.

In process
[D] compounds of the general formula (VI) 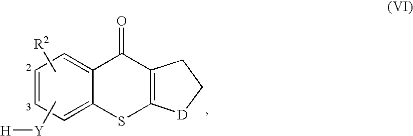
   in which
   H—Y— is located at one of positions 2 or 3 of the thiochromenone ring,
   R² and D have the meaning indicated above, and
   Y is O, S or NR⁶ in which R⁶ has the meaning indicated above, are reacted
   with compounds of the general formula (VII)
      R⁵-E-X¹ (VII),
   in which
   R⁵ and E have the meaning indicated above, and
   X¹ is a leaving group, preferably mesylate, tosylate or halogen, particularly preferably bromine or iodine,
   to give compounds of the general formula (Id) 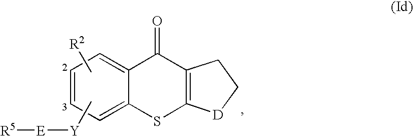
   in which
   R⁵-E-Y- is located at one of positions 2 or 3 of the thiochromenone ring, and
   R², R⁵, D, E and Y have the meaning indicated above,
   in inert solvents which are not changed under the reaction conditions, and these include halohydrocarbons such as methylene chloride, trichloromethane or 1,2-dichloroethane, ethers such as dioxane, tetrahydrofuran or 1,2-dimethoxyethane, or other solvents such as acetone, dimethylformamide, dimethylacetamide, 2-butanone or acetonitrile, preferably tetrahydrofuran, methylene chloride, acetone, 2-butanone, acetonitrile, dimethylfornarmide or 1,2-dimethoxyethane, in the presence of a base such as, for example, alkali metal carbonates such as cesium carbonate, sodium or potassium carbonate, or sodium or potassium methanolate, or sodium or potassium ethanolate or potassium tert-butoxide, or other bases such as sodium hydride, DBU, preferably potassium tert-butoxidecesium carbonate, DBU, sodium hydride, potassium carbonate or sodium carbonate, where appropriate in the presence of potassium iodide, preferably in a temperature range from room temperature up to refluxing of the solvents under atmospheric pressure.

The compounds of the general formula (VI) can be prepared from the appropriate precursors for example in analogy to process [M] or by the process described for preparing the compounds of the general formula (VIII).

The compounds of the general formula (VII) are known or can be prepared by known processes.

In process
[E] compounds of the general formula (VIII) 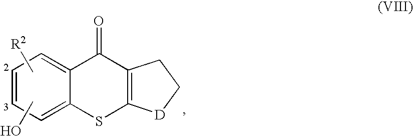
   in which
   hydroxyl is located at one of positions 2 or 3 of the thiochromenone ring, and
   R² and D have the meaning indicated above, are reacted
   with compounds of the general formula (IX)
      R¹⁹—X² (IX),
   in which
   R¹⁹ has the meaning indicated above,
   X² is halogen, preferably bromine,
   to give compounds of the general formula (Ie) 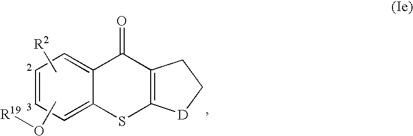
   in which
   R¹⁹—O— is located at one of positions 2 or 3 of the thiochromenone ring, and
   R², R¹⁹ and D have the meaning indicated above,
   in inert solvents which are not changed under the reaction conditions, and these include solvents such as dimethylformamide, dimethylacetamide, N-methylpyrrolidone, pyridine or hexamethylphosphoric triamide, preferably dimethylformamide, dimethylacetamide, pyridine or N-methylpyrrolidone, in the presence of a catalyst, preferably with copper catalysis, Cu(I) catalysts are particularly preferred, such as, for example, copper(I) iodide, where appropriate in the presence of a base such as, for example, alkali metal carbonates such as cesium carbonate, sodium or potassium carbonate or sodium or potassium methanolate, or sodium or potassium ethanolate or potassium tert-butoxide, or other bases such as DBU, pyridine or picoline, with preference for potassium carbonate or pyridine, preferably in a temperature range from 80° C. up to the refluxing of the solvents under atmospheric pressure.

Compounds of the general formula (VIII) are prepared by reacting compounds of the general formula (If) 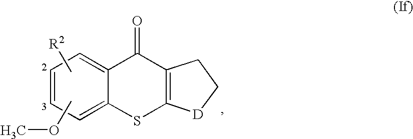
in which
methoxy is located at one of positions 2 or 3 of the thiochromenone ring,
and
R² and D have the meaning indicated above,
with hydrobromic acid in glacial acetic acid, preferably in a temperature range from 80° C. up to the refluxing of the solvents under atmospheric pressure.

Compounds of the formula (VIII) can also be prepared by reacting the compounds of the general formula (If) in an inert solvent, preferably dichloromethane, with boron trichloride or boron tribromide, where appropriate in the presence of a phase-transfer catalyst, preferably quaternary ammonium salts, particularly preferably tetrabutylammonium bromide or iodide, preferably in a temperature range from minus 20° C. up to the refluxing of the solvents under atmospheric pressure.

The compounds of the general formula (If) can be prepared from the appropriate precursors for example in analogy to process [M].

The compounds of the general formula (IX) are kmown or can be prepared by known processes.

In process
[F] compounds of the general formula (IX) 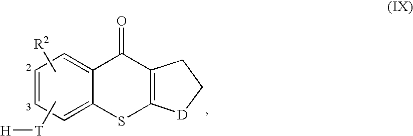
   in which
   H-T- is located at one of positions 2 or 3 of the thiochromenone ring,
   R² and D have the meaning indicated above, and
   T is O or NR²³ in which R²³ has the meaning indicated for R⁶,
   are reacted with compounds of the general formula (X) 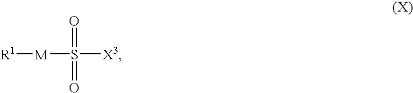
   in which
   R¹ has the meaning indicated above,
   M is a bond or NR²⁴ in which R²⁴ has the meaning indicated for R⁶,
   X³ is halogen, preferably bromine or chlorine,
   to give compounds of the general formula (Ig) 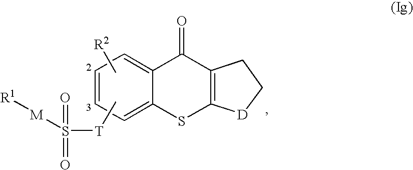
   in which
   R¹-M-SO₂-T- is located at one of positions 2 or 3 of the thiochromenone ring, and
   R¹, R², D, M and T have the meaning indicated above,
   in inert solvents or mixtures of solvents with water, which are not changed under the reaction conditions, and these include halohydrocarbons such as methylene chloride, trichloromethane or 1,2-dichloroethane, ethers such as diethyl ether, dioxane, tetrahydrofuran or 1,2-dimethoxyethane, hydrocarbons such as benzene, xylene or toluene, or other solvents such as acetone, dimethylformarnide, 2-butanone, acetonitrile or pyridine. It is likewise possible to employ mixtures of said solvents, where appropriate also with water. Mixtures with water are, for example, methylene chloride/water or dioxane/water, with preference for methylene chloride, methylene chloride/water, dioxanie, dioxane/water or tetrahydrofuran, where appropriate in the presence of a phase-transfer catalyst, preferably quatemary ammonium salts, particularly preferably tetrabutylammonium chloride or tetrabutylammonium bromide, where appropriate in the presence of a base such as, for example, alkali metal hydroxides such as sodium or potassium hydroxide, or alkali metal carbonates such as cesium carbonate, sodium or potassium carbonate, or sodium or potassium methanolate, or sodium or potassium ethanolate or potassium tert-butoxide, or other bases such as sodium hydride, DBU, triethylamine, dilsopropylethylamine or pyridine, with preference for pyridine or sodium hydroxide, preferably in a temperature range from room temperature up to the refluxing of the solvents under atmospheric pressure.

The compounds of the general formula (IX) can be prepared in analogy to the compounds of the general formula (VI).

The compounds of the general formula (X) are known or can be prepared by known processes.

In process
[G] compounds of the general formula (XI) 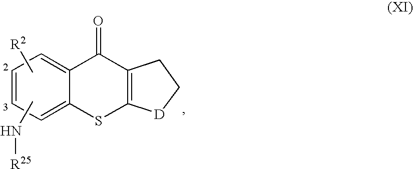
   in which
   R²⁵—NH— is located at one of positinos 2 or 3 of the thiochromenone ring,
   R²and D have the meaning indicated above, and
   R²⁵ has the meaning of R⁶ indicated above,
   are reacted with compounds of the general formula (XII)
      R¹-L-X³ (XI),
   in which
   R¹ has the meaning indicated above,
   X³ has the meaning indicated above,
   L is CO, SO₂ or NR¹²SO₂ or NR¹⁴CO, in which R¹² and R¹⁴ have the meaning indicated above,
   to give compounds of the general formula (Ih) 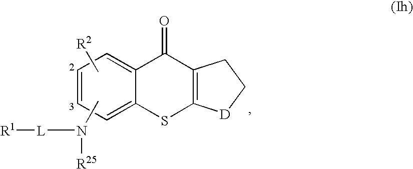
   in which
   R¹-L—NR²⁵— is located at one of positions 2 or 3 of the thiochromenone ring,
   and
   R¹, R², R²⁵, D and L have the meaning indicated above,
      in inert solvents which are not changed under the reaction conditions, and these include halohydrocarbons such as methylene chloride, trichloromethane or 1,2-dichloroethane, ethers such as diethyl ether, dioxane, tetrahydrofuran or 1,2-dimethoxyethane, or other solvents such as dimethylformamide, dimethylacetamide, acetonitrile or pyridine, preferably tetrahydrofuran, pyridine, chloroform or methylene chloride, in the presence of a base such as, for example, alkali metal carbonates such as cesium carbonate, sodium or potassium carbonate, or potassium tert-butoxide, or other bases such as sodium hydride, DBU, triethylamine or diusopropylethylamine, preferably triethylamine, diisopropylethylamine, cesium carbonate, potassium carbonate, sodium carbonate, pyridine and/or DMAP, preferably in a temperature range from 0° C. up to the refluxing of the solvents under atmospheric pressure.

The compounds of the general formula (XI) can be prepared in analogy to the compounds of the general formula (VI).

The compounds of the general formula (XII) are known or can be prepared by known processes.

In process
[H] compounds of the general formula (Ii) 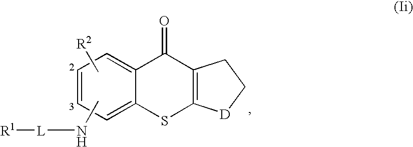
   in which
   R¹-L-NH— is located at one of positions 2 or 3 of the thiochromenone ring,
   and in which
   R¹, R², D and L have the meaning indicated above,
   are reacted with compounds of the general formula (XIII)
      R²⁶—X¹ (XIII),
   in which
   R²⁶ is (C₃-C₈)-cycloalkyl or optionally unsaturated (C₁-C₆)-alkyl which is optionally substituted by hydroxyl, phenyl, (C₁-C₆)-alkoxy, (C₁-C₆)-alkoxycarbonyl or (C₃-C₈)-cycloalkyl, where phenyl in turn may be substituted by halogen or (C₁-C₄)-alkyl,
   X¹ has the meaning indicated above,
   to give compounds of the general formula (Ij) 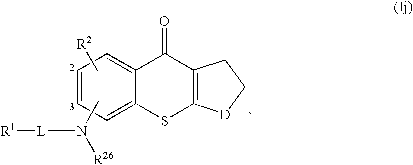
   in which
   R¹-L—NR²⁶— is located at one of positions 2 or 3 of the thiochromenone ring, and
   R¹, R², R²⁶, D and L have the meaning indicated above,
      in inert solvents which are not changed under the reaction conditions, and these include halohydrocarbons such as methylene chloride, trichloromethane, trichloroethane or 1,2-dichloroethane, ethers such as diethyl ether, dioxane, tetrahydrofuran or 1,2-dirnethoxyethane, or other solvents such as acetone, dimethylformamide, dimethylacetamide, N-methylpyrrolidone, 2-butanone or acetonitrile, preferably tetrahydrofuran, dimethylformamide, 2-butanone or acetone, in the presence of a base such as, for example, alkali metal carbonates such as cesium carbonate, sodium or potassium carbonate, or sodium or potassium methanolate, or sodium or potassium ethanolate or potassium tert-butoxide, or other bases such as sodium hydride, DBU, triethylamine or diisopropylethylarnine, preferably sodium hydride, DBU, potassium tert-butoxide or potassium carbonate, with, where appropriate, a phase-transfer catalyst, preferably tetrabutylammonium bisulfate, preferably in a temperature range from room temperature to 120° C. under atmospheric pressure.

The compounds of the general formula (Ij) can be prepared from the appropriate precursors in analogy to process [G].

The compounds of the general formula (XIII) are known or can be prepared by known processes.

In process
[I] compounds of the general formula (XIV) 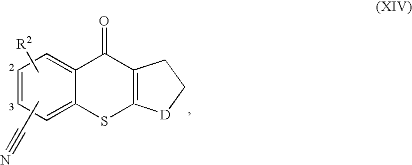
   in which
   cyano is located at one of positions 2 or 3 of the thiochromenone ring, and
   R² and D have the meaning indicated above,
   are reacted with an azide, preferably sodium azide, to give compounds of the general formula (Ik) 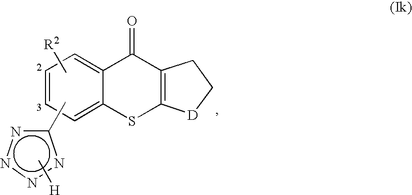
   in which
   the tetrazole residue is located at one of positions 2 or 3 of the thiochromenone ring, and
   R² and D have the meaning indicated above,
   in inert solvents, preferably toluene or xylene, in the presence of an acid, preferably triethylammonium hydrochloride, preferably in a temperature range from 80° C. up to the refluxing of the solvents under atmospheric pressure.

Where appropriate, compounds of the formula (Ik) are reacted in a second step with compounds of the general formula (XV)
R²⁷—X³ (XV),
in which
R²⁷ is (C₁-C₆)-alkyl or (C₁-C₆)-acyloxymethyl, and
X³ has the meaning indicated above,
to give compounds of the general formula (II) 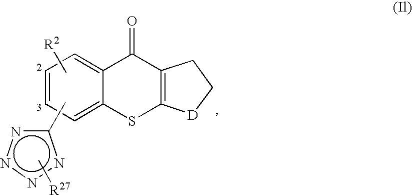
in which
the heterocycle is located at one of positions 2 or 3 of the thiochromenone ring, and
R², R²⁷ and D have the meaning indicated above,
in inert solvents which are not changed under the reaction conditions, and these include halohydrocarbons such as methylene chloride, trichloromethane, trichloroethane or 1,2-dichloroethane, ethers such as diethyl ether, dioxane, tetrahydrofuran or 1,2-dimethoxyethane, or other solvents such as acetone, dimethylformamide, dimethylacetamide, 2-butanone or acetonitrile, preferably tetrahydrofuran, methylene chloride, acetone, 2-butanone or dimethylformamide, in the presence of a base such as, for example, alkali metal carbonates such as cesium carbonate, sodium or potassium carbonate, or sodium or potassium methanolate, or sodium or potassium ethanolate or potassium tert-butoxide, or other bases such as sodium hydride, DBU, triethylamine or diisopropylethylamine, preferably sodium hydride or DBU, preferably in a temperature range from 0° C. up to the refluxing of the solvents under atmospheric pressure.

Process [I] is also used for appropriate variations of substituents.

Compounds of the general formula (XIV) are prepared for example by reacting compounds of the general formula (II) with Zn(II) cyanide in the presence of a catalyst in a suitable solvent, and these include ethers such as dioxane, 1,2-dimethoxyethane or diethylene glycol dimethyl ether, or other solvents such as dimethylformamide, dimethylacetamide, N-methylpyrrolidone or acetonitrile, with particular preference for dimethylformamide or dimethylacetamide, preferably in a temperature range from 60° C. to 160° C. under atmospheric pressure.

The reaction is preferably carried out with catalysis by palladium (D. M. Tschaen, et al., Synth. Commun. 1994, 24, 887; F. Jin, N. P. Confalone, Tetrahedron Lett. 2000, 41, 3271), examples of particularly preferred catalysts being tetrakis(tri-phenylphosphine)palladium(0) or tris(dibenzylideneacetone)dipalladium in the presence of 1,1′-[bis(diphenylphosphino)ferrocene] and zinc.

The compounds of the general formula (XV) are known or can be prepared by known processes.

In process
[J] compounds of the general formula (XIV) are reacted in the first step with hydroxylamine and subsequently with chloroformic esters, preferably 2-ethylhexyl chloroformate in two further stages to give compounds of the general formula (Im) 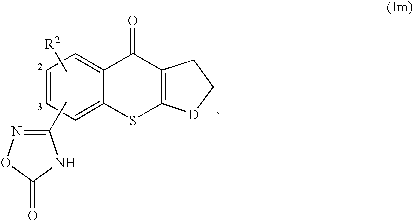
   in which
   the heterocycle is located at one of positions 2 or 3of the thiochromenone ring, and
   R² and D have the meaning indicated above.

The first reaction step is carried out in inert solvents which are not changed under the reaction conditions, and these include ethers such as diethyl ether, dioxane, tetrahydrofuran, 1,2-dimethoxyethane or diethylene glycol dimethyl ether, hydrocarbons such as benzene, xylene or toluene, or other solvents such as dimethylformamide, dimethylacetamide, dimethyl sulboxide, pyridine or hexamethylphosphoric triamide, preferably dimethyl sulfoxide, preferably in a temperature range from room temperature to 100° C. under atmospheric pressure.

The second reaction step is carried out in inert solvents which are not changed under the reaction conditions, and these include halohydrocarbons such as methylene chloride, trichloromethane, 1,2-dichloroethane or tiichloroethylene, ethers such as diethyl ether, dioxane, tetrahydrofuran, glycol dimethyl ether or diethylene glycol dimethyl ether, or other solvents such as dimethylforinamide, dimethylacetainide, 1,2-dimethoxyethane, pyridine or N-methylpyrrolidine, preferably dimethyformamide, where appropriate in the presence of a base such as, for example, alkali metal carbonates such as cesium carbonate, sodium or potassium carbonate, or other bases such as sodium hydride, DBU, tiiethylamine, diisopropylethylamine or pyridine, preferably pyridine, preferably in a temperature range from 0° C. to room temperature under atmospheric pressure.

The third reaction step is carried out in inert solvents, preferably xylene, preferably in a temperature range from 10° C. up to the refluxing of the solvents under atmospheric pressure.

Process [J] is also used for appropriate variations of substituents.

In process
[K] compounds of the general formula (XV) are reacted first with a suitable acid to give compounds of the general formula (XVI) 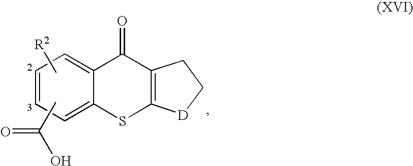
   in which
   carboxyl is located at one of positions 2 or 3 of the thiochromenone ring, and
   R² and D have the meaning indicated above,
   preferably in a temperature range from room temperature up to the refluxing of the solvents under atmospheric pressure.

Acids which are generally suitable are trifluoroacetic acid, sulfuric acid, hydrogen chloride, hydrogen bromide and acetic acid or mixtures thereof, where appropriate with addition of water. Sulfuric acid is particularly preferably employed.

The compounds of the general formula (XVI) are then reacted with compounds of the general formula (XVII) 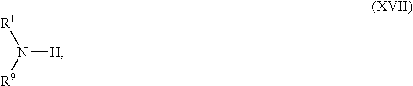
in which
R¹ and R⁹ have the meaning indicated above,
to give compounds of the general formula (In) 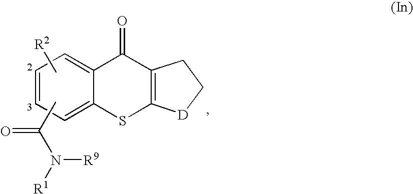
in which
R¹R⁹N—CO— is located at one of positions 2 or 3 of the thiochromenone ring, and
R¹, R², R⁹ and D have the meaning indicated above.

Solvents preferred for the reaction with compounds of the general formula (XVII) are dichloromethane, tetrahydrofuran or dimethylformamide. The reaction takes place in particular at room temperature. Aids preferably employed for this reaction are usual condensing agents such as carbodiimides, e.g. N,N-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium-3-sulfate or 2-tert-butyl-5-methylisoxazolium perchlorate, or acylaiino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri-(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylammno)phosphonium hexafluorophosphate (BOP). Bases employed are alkali metal carbonates, e.g. sodium or potassium carbonate or bicarbonate, or organic bases such as trialkylamines, e.g. triethylamine, N-methylmorpholine, N-methylpiperidine, 4-dimethylaminopyridine or diisopropylethylarnine. Particular preference is given to the combination of N-cyclohexylcarbodiimide-N′-propyloxym ethyl-polystyrene (PS-carbodiimide) and 1-hydroxybenzotriazole (HOBt) and to the combination of benzotriazol-1-yloxytris(dimethylamino)phosphonium hexafluorophosphate (BOP) and triethylamine.

The compounds of the general formula (XVII) are known or can be prepared by known processes.

In process
[L] compounds of the general formula (XI) are reacted with compounds of the general formula (XVIII) 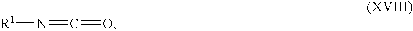
   in which
   R¹ has the meaning indicated above,
   to give compounds of the general formula (Io) 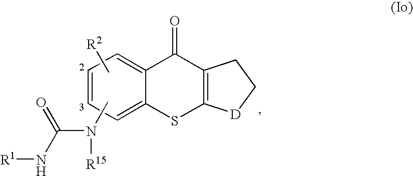
   in which
   R¹—NH—CO—NR¹⁵— is located at one of positions 2 or 3 of the thiochromenone ring, and
   R¹, R², R¹⁵ and D have the meaning indicated above,
   in inert solvents which are not changed under the reaction conditions, and these include halohydrocarbons such as methylene chloride, trichloromethane or 1,2-dichlorethane, ethers such as diethyl ether, dioxane, tetrahydrofuran or glycol dimethyl ether, hydrocarbons such as benzene, xylene or toluene, or other solvents such as acetone, dimethylforrnamide, dimethylacetamide, 2-butanone, acetonitrile or pyridine, preferably tetrahydrofuran, pyridine or methylene chloride, where appropriate in the presence of a base such as alkali metal carbonates such as cesium carbonate, sodium or potassium carbonate, or potassium tert-butoxide, or other bases such as sodium hydride, DBU, triethylamine, pyridine or diisopropylethylamine, preferably triethylamine, pyridine or diisopropylethylarmine, preferably in a temperature range from room temperature up to the refluxing of the solvents under atmospheric pressure.

The compounds of the general formula (XVIII) are known or can be prepared by known processes.

In process
[M] compounds of the general formula (XIX) 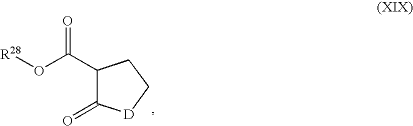
   in which
   R²⁸ is (C₁-C₆)-alkyl or benzyl, preferably ethyl or methyl,
   and
   D has the meaning indicated above,
   are reacted with compounds of the general formula (XX) 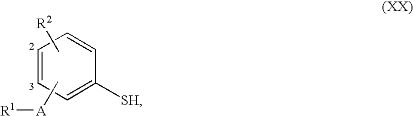
   in which
   R¹, R² and A have the meaning indicated above,
   in polyphosphoric acid to give compounds of the general formula (I) 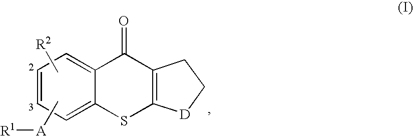
   in which
   R¹-A- is located at one of positions 2 or 3 of the thiochromenone ring, and
   R¹, R², A and D have the meaning indicated above,
   preferably in a temperature range from 70° C. to 110° C. under atmospheric pressure.

Compounds of the formula (XX) which are preferably employed are those which are stable under the reaction conditions used.

The cyclic keto esters (XIX) are known or can be prepared by generally known processes, e.g. by Claisen condensation of the cyclic ketones with dialkyl carboxylates (e.g. S. J. Rhoads et al., Tetrahedron 1963, 19, 1625), or with dialkyl oxalates followed by decarboxylation (e.g. L. Re, H. Schinz, Helv. Chim. Acta 1958, 41, 1695).

The cyclic ketones are known or can be prepared by generally known processes, e.g. from the corresponding cyclo-2-alkenones or 1,3-diketones.

The compounds and (XX) are known or can be prepared by known methods. Dimercaptobenzenes substituted on one sulfur can be prepared for example by the method of J. Campbell et al. J. Org. Chem. 1964, 29, 1830-1833; Rumpf et al., Bull. Soc. Chim. Fr. 1940, 7, 632. 3-Sulfonylthiophenols can be prepared for example by the method of Melloni et al., J. Chem. Soc., Perkin Trans. 1972, 1, 218; Borwell et al. 1953, 75, 6019. 3-Mercaptobenzenesulfonic acids can be prepared by the method of H. Kawai et al., Chem. Pharm. Bull. 1991, 39, 1422-1425.

In process
[N] compounds of the general formula (Ip) 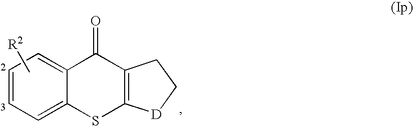
   in which
   R² and D have the meaning indicated above,
   are reacted with compounds of the general formula (XXI) 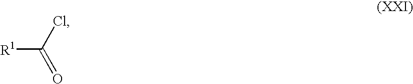
   in which
   R¹ has the meaning indicated above,
   to give compounds of the general formula (Iq) 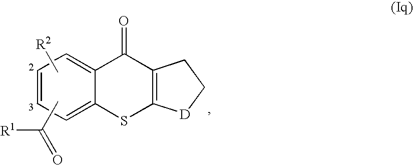
   in which
   R¹CO— is located at one of positions 2 or 3 of the thiochromenone ring, and
   R¹, R² and D have the meaning indicated above,
   in inert solvents which are not changed under the reaction conditions, and these include halohydrocarbons such as tetrachloroethane, 1,2-dichloroethane or chlorobenzene, or other solvents such as dimethylformamide, preferably dimethylformamide, preferably in the presence of aluminum trichloride, preferably under Friedel-Crafts acylation conditions analogous to the conditions described in the literature (O. Diouf et al., Eur. J. Med. Chem. 1995, 30, 715-719; S. Yous et al., J. Org. Chem. 1994, 59, 1574-1576).

The corresponding methyl ketones [R¹ is methyl in (Ip)] can additionally be obtained from the compounds of the general formula (II) preferably by means of a Heck or Stille reaction by methods known from the literature (W. Cabri et al., Tetrahedr. Lett. 1991, 32, 1753-1756; M. Kosugi et al., Bull. Chem. Soc. Jpn. 1987, 60, 767-768.). The methyl ketones can, where appropriate, be further derivatized after bromination of the methyl group under standard conditions usual for this purpose.

The compounds of the general formula (Ip) can be prepared from the appropriate precursors for example in analogy to process [M].

The compounds of the general formula (XXIII) are known or can be prepared by known processes.

In process
[O] compounds of the general formula (Ir) 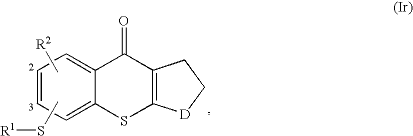
   in which
   R¹—S— is located at one of positions 2 or 3 of the thiochromenone ring, and
   R¹, R² and D have the meanings indicated above,
   are reacted with oxidizing agents such as, for example, m-chloroperbenzoic acid, to give compounds of the general formula (Is) 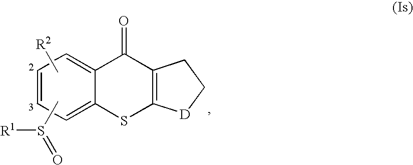
   in which
   R¹—SO— is located at one of positions 2 or 3 of the thiochromenone ring, and
   R¹, R² and D have the meaning indicated above,
      for example under the conditions described in the literature (P. Margaretha et al., Helv. Chim. Acta 1979, 62, 1978-1979; P. Bendazzoli et al., Tetrahedr. Lett. 1993, 34, 2975-2978.).

The compounds of the general formula (Ir) can be prepared from the appropriate precursors for example in analogy to process [M].

In process
[P] compounds of the general formula (XXII) 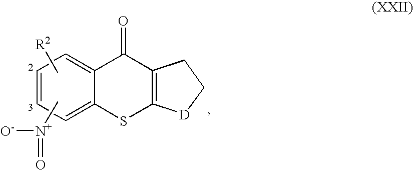
   in which
   nitro is located at one of positions 2 or 3 of the thiochromenone ring, and
   R² and D have the meaning indicated above,
   are reacted with the appropriate sulfinic acid salts to give compounds of the general formula (It) 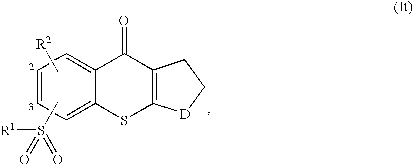
   in which
   R¹—SO₂— is located at one of positions 2 or 3 of the thiochromenone ring, and
   R¹, R² and D have the meaning indicated above,
   preferably under the conditions described in the literature (W. Fischer et al., Helv. Chim. Acta 1985, 68, 854-59.).

The compounds of the general formula (XXII) can be prepared from the appropriate precursors for example in analogy to process [M].

In process
[Q] compounds of the general formula (Iu) 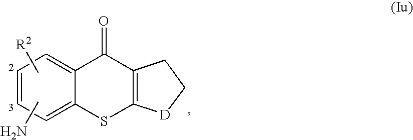
   in which
   amino is located at one of positions 2 or 3 of the thiochromenone ring, and
   R² and D have the meaning indicated above,
   are converted after azotization into the corresponding sulfonic acids, and the latter are subsequently reacted with compounds of the general formula (XXIII)
      R¹—U—H (XXIII),
   in which
   R¹ has the meaning indicated above, and
   U is O or NR¹⁰ in which R¹⁰ has the meaning indicated above,
   to give compounds of the general formula (Iv) 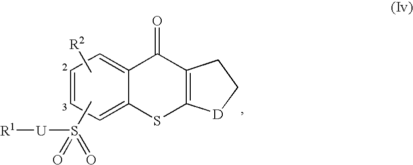
   in which
   R¹—U—SO₂— is located at one of positions 2 or 3 of the thiochromenone ring, and
   R¹, R², D and U have the meaning indicated above.

The first reaction step is preferably carried out under the conditions described in the literature (Meerwein et al., Chem. Ber. 1957, 90, 841-51; Polak et al., Recl. Trav. Chim. Pays-Bas 1910, 29, 423.).

In the second stage, the sulfonic acids can be converted, where appropriate via their chlorides, by esterification or amination by methods known to the skilled worker into the corresponding sulfonates or sulfonamides.

The compounds of the general formula (It) are prepared as described for compounds of the general formula (XI).

The compounds of the general formula (XXIII) are known or can be prepared by known processes.

Amines in side chains can also be prepared by reductive amination of corresponding aldehydes with appropriate amines. Examples which may be mentioned are:

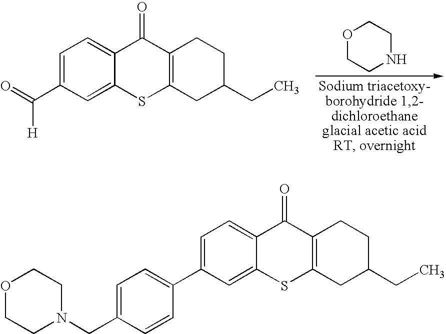

The processes described above can be illustrated by way of example by the following formula diagrams:

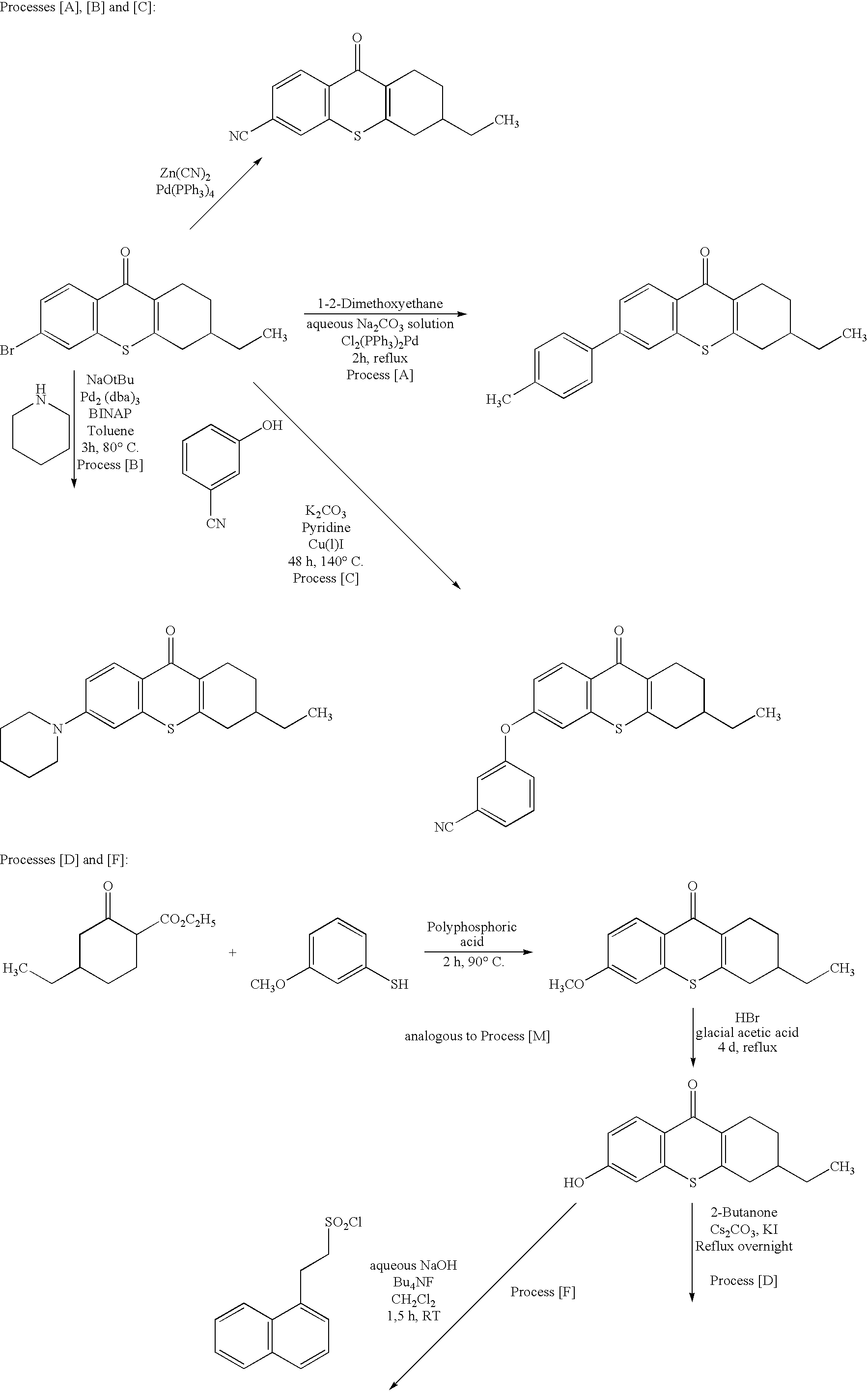

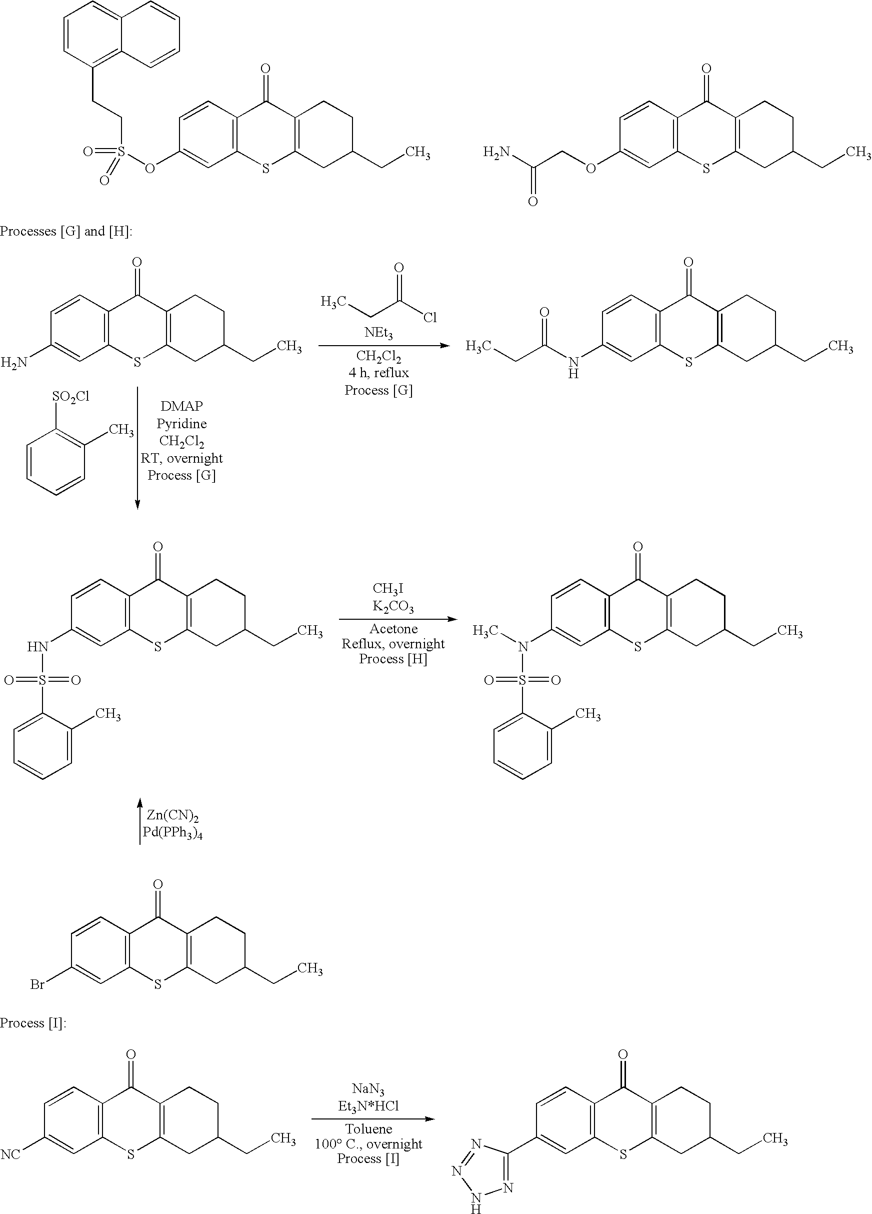

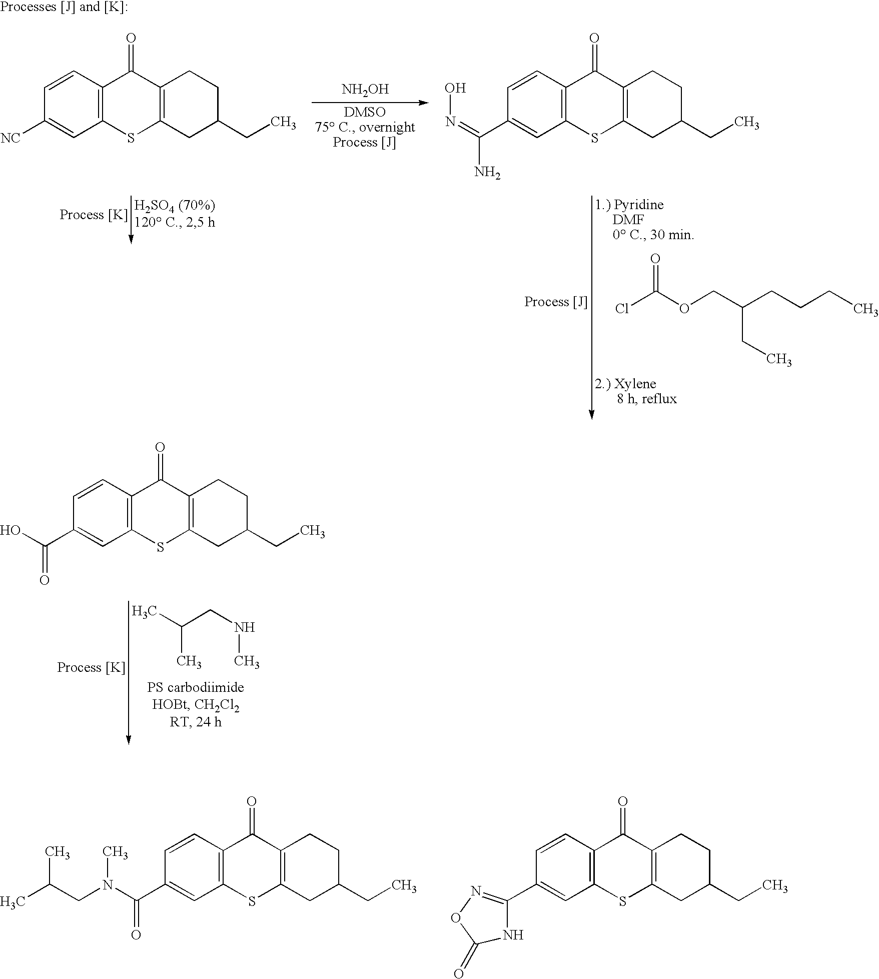

The compounds of the invention of the general formula (I) are suitable for use as medicaments in the treatment of humans and animals.

The compounds of the invention show a valuable range of pharmacological effects which could not have been predicted.

They are distingished as mGluR1 receptor antagonists.

The compounds of the invention can, by reason of their pharmacological properties, be employed alone or in combination with other medicaments for the treatment and/or prevention of neuronal damage or disorders connected with derangement of the physiological or pathophysiological states of the glutamatergic system in the central and peripheral nervous system.

For the treatment and/or prevention of neuronal damage, for example, by ischemic, thromb- and/or thromboembolic, and haemorrhagic stroke, conditions following direct and indirect injuries in the region of the brain and of the skull. Also for the treatment and/or prevention of cerebral ischemias after all surgical procedures on the brain or peripheral organs or body parts and associated or preceding conditions of a pathological or allergic nature which may lead primarily and/or secondarily to neuronal damage.

The compounds of the invention are likewise also suitable for the therapy of primary and/or secondary pathological conditions of the brain, for example during or after cerebral vasospasms, hypoxia and/or anoxia of an origin not previously mentioned, perinatal asphyxia, autoimmune diseases, metabolic and organic disorders which may be associated with damage to the brain, and damage to the brain as a result of primary brain disorders, for example epilepsy and atherosclerotic and/or arteriosclerotic changes. For the treatment of chronic or psychiatric disorders such as, for example, depression, neurodegenerative disorders such as, for example, Alzheimer's, Parkinson's or Huntington's disease, multiple sclerosis, amyotrophic lateral sclerosis, neurodegeneration owing to acute and/or chronic viral or bacterial infections and multi-infarct dementia.

They can moreover be employed as medicaments for the treatment of dementias of varying origins, cognitive impairments in the elderly, memory impairments, spinal cord injuries, states of pain, anxiety states of varying origins, drug-related Parkinson's syndrome, psychoses (such as, for example, schizophrenia), cerebral edema, neuronal damage following hypoglycemia, emesis, nausea, obesity, addictive disorders and withdrawal symptoms, CNS-mediated convulsions, sedation and movement disorders.

The compounds of the invention of the general formula (I) can additionally be used to promote neuronal regeneration in the post-acute phase of cerebral injuries or chronic disorders of the nervous system.

The compounds of the invention can be employed alone or in combination with other medicaments for the prophylaxis and treatment of acute and/or chronic pain (for a classification, see “Classification of Chronic Pain, Descriptions of Chronic Pain Syndromes and Definitions of Pain Terms”, 2nd edition, Meskey and Begduk, Editors; IASP-Press, Seattle, 1994) and neurodegenerative disorders, especially for the treatment of cancer-induced pain and chronic neuropathic pain like, for example, that associated with diabetic neuropathy, post-herpetic neuralgia, peripheral nerve damage, central pain (for example the consequence of cerebral ischemia) and trigeminal neuralgia, and other chronic pain such as, for example, lumbago, backache (low back pain) or rheumatic pain. These substances are in addition also suitable for the therapy of primary acute pain of any origin and of secondary states of pain resulting therefrom, and for the therapy of states of pain which were formally acute and have become chronic.

They are preferably employed as medicaments for the treatment and/or prophylaxis of states of pain and neurodegenerative disorders.

Modulation of substances at the metabotropic glutamate receptor (direct or indirect influencing of the efficiency of coupling of the glutamate receptor to the G proteins) can be examined on primary cultures of granule cells from the cerebrum. Electrophysiological measurements on these cell cultures in the cell attached mode show that L-type Ca²⁺ channels in this preparation are activated by mGluR1 glutamate receptors (J. Neurosci. 1995, 15, 135), whereas they are blocked by group II receptors J. Neurosci. 1994, 14, 7067-7076). The modulating effect of pharmacological test substances on glutamate receptors can be checked by an appropriate experimental arrangement. A detailed examination of the subtype specificity under controlled conditions is possible on Xenopus oocytes through injection of the appropriate mGluR subtype DNA (WO 92/10583).

The in vitro effect of the compounds of the invention on mGluR1 receptors can be shown by the following biological assays:

1. Determination of the activity on the mGluR1 receptor

mGluR1 receptor-expressing CHO cells are seeded in 96-well plates (Greiner, Frickenhausen, Germany) (20 000 cells/well) and cultivated in ultra CHO medium (Bio-Whittaker, Walkersville, Md., 10% fetal calf serum) for one day. The substances to be tested are initially dissolved in a concentration of 10⁻² M in 100% DMSO and then diluted to the desired concentration with the culture medium. After being washed once with cell culture medium, the cells are incubated with 1 mM glutamate and simultaneously with the substance to be tested at 37° C. for 4 h. The medium is then aspirated off and the cells are lysed with 75 μl of Triton-luciferase buffer (1% Triton X 100, 10% glycerol, 2 mM DTT, 25 mM Na2HPO4, 25 mM TRIS), (530 μM ATP, 470 μM luciferin, 270 μM CoA, 20 mM tricine, 2.67 mM MgSO4, 33.3 mM DTT, 0.1 mM EDTA, pH 7.8). The luminescence is measured after one minute using a camera (Hamamatsu, Japan). Cells incubated only with glutamate show a marked increase in the luminescence compared with controls, while mGluR1 receptor antagonists reduce this concentration-dependently to below the initial luminscence.

The following examples showed the following effect in the abovementioned assay:

TABLE 1

| Example | IC₅₀ [nM] |
|---|---|
| 1-3 | 9 |
| 1-10 | 97 |
| 1-27 | 23 |
| 1-29 | 21 |
| 1-31 | 31 |
| 1-34 | 81 |
| 3-21 | 22 |
| 3-22 | 13 |
| 3-23 | 28 |
| 3-49 | 35 |
| 3-55 | 35 |
| 4-08 | 25 |

The suitability of the compounds of the invention for the treatment of states of pain, especially states of neuropathic pain, can be shown in the following animal models:

2. Axotomy of sciatic branches in the rat (chronic pain model)

Under pentobarbital anesthesia, the trifurcation of a sciatic nerve is exposed, and the peroneal and tibial branches are axotomized after the nerves have been ligated proximal of the axotomy site. Control animals undergo a sham operation. After the operation, the axotomized animals develop chronic mechanical allodynia and thermal hyperalgesia.

The mechanical allodynia is tested, comparing with sham-operated animals, with the aid of a pressure transducer (electronic von Frey anesthesiometer, ITC Inc.-Life Science Instruments, Woodland Hills, Calif., USA).

The thermal hyperalgesia can be detemirned by measuring the latency time within which a rat removes a paw from the area of a radiant heat source (plantar test, Ugo Basile (Milan)).

The substance is administered by various administration routes (i.v., i.p., orally, i.t., i.c.v., transdermally) at various times before the pain testing.

3. Ligature of the sciatic nerve in the rat according to Bennett and Xie, 1988 (chronic pain model)

Bennett und Xie: A peripheral mononeuropathy in the rat that produces disorders of pain sensation like those seen in man. Pain 1988, 33, 87-107.

Under pentobarbital anesthesia, the sciatic nerve is exposed unilaterally and ligated (4 ligatures approximately 1 mm apart, proximal to the trifurcation of the nerve). Control animals undergo a sham operation. After the operation, the ligated animals develop a chronic mechanical and thermal hyperalgesia. The hyperalgesia is tested, comparing with sham-operated animals, with the aid of a pressure transducer (mechanical hyperalgesia; electronic von Frey anesthesiometer, IITC Inc.-Life Science Instruments, Woodland Hills, Calif., USA) or an infrared source (thermal hyperalgesia; Plantar Test, Hugo Basile Inc., Comerio, Italy).

The substance is administered by various administration routes (i.v., i.p., orally, i.t., i.c.v., transdermally) at various times before the pain testing.

The suitability of the compounds of the invention for the treatment of states of pain, especially inflammation-related states of pain, can be shown in the following animal model.

4. Model of acute inflammatory pain (carrageenin model) in rats.

This method, which tests analgesic effect in rats suffering from inflammatory pain, follows the description of Winter et al. (Proc. Soc. Exp. Biol. Med., 1962, 111, 544-547.

Rats receive subplantar injection into the right rear paw of a suspension of carrageenin (0.75 mg per paw in 0.05 ml of physiological saline). Two hours later, the rats undergo successive thermal and tactile stimulation both on the noninflamed and on the inflamed rear paw.

The apparatus for thermal stimulation (Ugo Basile, Ref.: 7371) consists of 6 individual Plexiglas boxes (17×11×13 cm) placed on an elevated glass plate. A rat is placed in the box for 10 min for habituation. A movable infrared source (setting 20) is then focused under the noninflamed and the inflamed rear paw, and the latency time until the paw is withdrawn are recorded automatically. The withdrawal of the paw interrupts the reflected beam and thus automatically switches off the counter and light source. To avoid tissue damage, the test is stopped after 45 s even if no paw-withdrawal is recorded.

For the tactile stimulation, the animal is placed in a Plexiglas box (17×11×13 cm) whose base consists of a wire mesh. The tip of an electronic Von-Frey filament (Bioseb, Model 1610) is pushed with increasing pressure against the uninflamed and the inflamed rear paw, and the force required to bring about withdrawal of the paw is automatically recorded.

The testing is carried out three times and the average force per paw is calculated as the result for each animal.

12 rats are investigated per group: male Wistar (Han) rats, 180-220 g. The test is carried out blind.

Morphine (8 mg/kg i.p.) and acetylsalicylic acid (256 mg/kg i.p.), administered under the same experimental conditions, serve as reference substances.

Data analysis takes place by comparing the treated groups with the corresponding controls by means of the unpaired Student's test.

The novel active substances can be converted iin a known manner into conventional formulations such as tablets, coated tablets, pills, granules, aerosols, syrups, emulsions, suspensions and solutions, using inert, nontoxic, pharmaceutically suitable carriers or solvents. In these, the therapeutically active compound should be present in each case in a concentration of about 0.5 to 90% by weight of the complete mixture, i.e. in amounts which are sufficient to achieve the stated dose range.

The formulations are produced for example by extending the active substances with solvents and/or carriers, where appropriate with use of emulsifiers and/or dispersants, it being possible, for example when water is used as diluent, where appropriate to use organic solvents as auxiliary solvents.

Administration takes place in a conventional way, preferably orally, transdermally or parenterally, especially perlingually or intravenously. However, it can also take place by inhalation through the mouth or nose, for example with the aid of a spray, or topically via the skin.

It has generally proved advantageous to administer amounts of about 0.001 to 10 mg/kg, on oral administration preferably about 0.005 to 3 mg/kg, of body weight to achieve effective results.

It may nevertheless be necessary where appropriate to deviate from the amounts mentioned, in particular as a function of the body weight and the mode of administration, on the individual response to the medicament, the nature of its formulation and the time or interval over which administration takes place. Thus, in some cases it may be sufficient to make do with less than the aforementioned minimum amount, whereas in other cases the upper limit mentioned must be exceeded. Where larger amounts are administered, it may be advisable to distribute these in a plurality of single doses over the day.

### Abbreviations

abs. absolute
Ac acetyl
acac acetylacetonyl
AIBN α,α-azobis(isobutyronitrile)
Aloc allyloxycarbonyl
aqueous aqueous
aq. aqueous
9-BBN 9-borabicyclo[3.3.1]nonane
Bn benzyl
Boc tert-butoxycarbonyl
Bom benzyloxymethyl
BOP benzotriazol-1-yloxy-tris(dimethylamino)phosphonium hexafluorophosphate
b.p. boiling point
Bu butyl
Bz benzoyl
CAN cerium ammonium nitrate
Cbz benzyloxycarbonyl
CDI N,N′-carbonyldiimidazole
cf. compare
CH cyclohexane
conc. concentrated
Cp cyclopentadienyl
cryst. crystalline/crystallized
CSA 10-camphorsulfonic acid
Dabco 1,4-diazabicyclo[2.2.2]octane
DAST diethylaminosulfur trifluoride
DBN -1,5-diazabicyclo[4.3.0]non-5-ene
DBU 1,8-diazabicyclo[5.4.0]undec-7-ene
DCC N,N′-dicyclohexylcarbodiimide
DCE 1,2-dichloroethane
DCI direct chemical ionization (in MS)
DCM dichloromethane
DDQ 2,3-dichloro-5,6-dicyano-1,4-benzoquinone
DEAD diethyl azodicarboxylate
d.e. diastereomeric excess
decomp. decomposition
dil. diluted
DHP 3,4-dihydro-2h-pyran
DIAD diisopropyl azodicarboxylate
DIBAH diisobutylalurninum hydride
DIC diisopropylcarbodiimide
DEEA N,N-diisopropylethylamine
dist. distilled
DMA N,N-dimethylacetamide
DMAP 4-N,N-dimethylaminopyridine
DME 1,2-dimethoxyethane
DME N,N-dimethylformamide
DMPU N,N′-dimethylpropylene urea
DMSO dimethyl sulfoxide
DNPH 2,4-dinitrophenylhydrazine
DPPA diphenylphosphoryl azide
EA ethyl acetate
EDC N′-(3-dimethylaminopropyl)-n-ethylcarbodiimide x hc1
e.e. enantiomeric excess
EI electron impact ionization (in ms)
eq equivalent(s)
ESI electrospray ionization (in ms)
Et ethyl
Fmoc fluorenylmethoxycarbonyl
Fr. fraction
GC gas chromatography
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HMDS 1,1,1,3,3,3-hexamethyldisilazane
HMPA or HMPT hexamethylphosphoric triamide
HOBt -hydroxy-1h-benzotriazole x h₂o
HOSu N-hydroxysuccinimide
HPLC high pressure, high performance liquid chromatography
Im irnidazol-1-yl
IR infrared spectroscopy
LAH lithium aluminum hydride
LC-MS coupled liquid chromatography—mass spectroscopy
LDA lithium-N,N-diisopropylamide
LiHMDS lithium-N,N-bistrimethylsilylamide
liq. liquid
Lit. literature (reference)
m meta
mCPBA meta-chloroperbenzoic acid
Me methyl
MEK methyl ethyl ketone
MEM methoxyethoxymethyl
MOM methoxymethyl
m.p. melting point
MPLC medium pressure liquid chromatography
Ms methanesulfonyl (Mesyl)
MS mass spectroscopy
MTBE methyl tert-butyl ether
MW molecular weight
NBS N-bromosucciniride
NCS N-chlorosuccinimide
NIS N-iodosuccinimide
NMM N-methylmorpholine
NMO N-methylmorpholine n-oxide
NMR nuclear magnetic resonance spectroscopy
o ortho
p para
p.A. analytical grade
PCC pyridinium chlorochromate
PDC pyridinium dichromate
Pfp pentafluorophenyl
Ph phenyl
Piv pivaloyl
PMB p-methoxybenzyl
PNB p-nitrobenzyl
PPA polyphosphoric acid
PPTS pyridinium p-toluene sulfonate
Pr propyl
prec. precipitate
PS polystyrene (resin)
py pyridine
PyBOP benzotriazol-1-yloxy-tris(pyrrolidino)phosphonium hexafluorophosphate
RF reflux
R_{f} retention index (in TLC)
RP reverse phase (in HPLC)
RT room temperature
Rₜ retention time (in HPLC)
sat. saturated
SEM ²-(trimethylsilyl)ethoxymethyl
sol. solution
subl. sublime
TBAF tetrabutylammonium fluoride
TBAI tetrabutylammonium iodide
TBDMS tert-butyldimethylsilyl
TBDPS tert-butyldiphenylsilyl
TBTU o-(benzotriazol-1-yl)-N,N,N′,N′-tetramethyluronium tetrafluoroborate
TEA triethylamine
techn. technical
Teoc 2-(trimethylsilyl)ethoxycarbonyl
TES triethylsilyl
Tf trifluoromethanesulfonyl
TFA trifluoroacetic acid
TFAA trifluoroacetic anhydride
TfOH trifluoromethanesulfonic acid
THF tetrahydrofuran
THP tetrahydropyranyl
TIPS triisopropylsilyl
titr. titrated
TLC thin layer chromatography
TMEDA N,N,N′N′-tetramethylethylenediamine
TMOF trimethyl orthoforrnate
TMS trimethylsilyl
TPP triphenylphosphine
TPPO triphenylphosphine oxide
Trt trityl
Ts p-toluenesulfonyl (Tosyl)
TsOH p-toluenesulfonic acid
v/v volume to volume ratio (of a solution)
Vol. volume
w/w weight to weight ratio (of a solution)
Z benzyloxycarbonyl

### STARTING COMPOUNDS I

The retention time of starting compounds and preparation examples was determined by HPLC under the following conditions.

HPLC Method:

Column: Chromasil C18 60*2; volume injected 1.00 μl; flow rate: 0.75 ml/min; eluent: A=5 ml HClO₄/l H₂O, B=CH₃CN; gradient [t(min): A/B]: 0.5: 98/2; 4.5: 10/90; 6.5: 10/90; 6.7: 98/2; 7.5: 98:2.

Preparative HPLC method: Reverse phase, ACN/water gradient; column: GROM-SIL 120 ODS-4 HE 10 μm, 250* 30 mm

### EXAMPLE I-1

### 6-Bromo-3-ethyl-1,2,3,4-tetrahydro-9H-thioxanthen-9-one

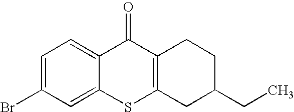

The reaction is carried out in an apparatus with mechanical stirrer under argon, and off-gases are passed through chlorine bleach solution.

110 g of polyphosphoric acid are introduced under a strong argon stream and stirred at RT for 15 min under a strong argon stream, and the reaction flask is then heated with a hot-air blower for about 5 min until a lower viscosity is evident. It is allowed to cool again, 10.0 g (51.3 mmol) of 3-bromothiophenol and 11.2 g (56.4 mmol) of ethyl 4-ethyl-2-oxocyclohexylcarboxylate are added with disposable syringes, and the mixture is stirred at normal temperature for some minutes. The honey-like reaction mixture changes color to beige and is heated to 90° C. and then stirred at this temperature for 2 h.

For working up, the mixture is allowed to cool to normal temperature, and firstly about 300 g of ice and 50 ml of water are added and, after 10 min, 300 ml of dichloromethane are also added, and the mixture is stirred at normal temperature for 20 min. After phase separation, the aqueous phase is extracted with dichloromethane (3×330 ml). The organic phases are washed twice with 200 ml of 1N sodium hydroxide solution each time and once with 100 ml of saturated brine, dried over magnesium sulfate and concentrated in a rotary evaporator.

The 17.8 g of crude product obtained in this way are recrystallized several times from about 150 ml of petroleum ether p.A. (60°-80°) each time to move the 8-bromo regioisomer.

5.55 g (34%) of the target compound are obtained in this way.

MS (EI+): 322 (M);

¹H-NMR (400 MHz, CDCl₃): δ=0.99 (t, J=7.5 Hz; 3H); 1.33-1.46 (m, 3H); 1.66-1.76 (m, 1H); 2.01-2.07 (m, 1H); 2.37-2.55 (m, 2H); 2.68-2.73 (m, 1H); 2.88-2.93 (m, 1H); 7.55-7.57 (dd, J=8.5 Hz, 2 Hz; 1H); 7.66 (d, J=2 Hz, 1H); 8.34 (d, J=8.5 Hz; 1H).

The following were prepared in analogy to the method of Example I-1:

### EXAMPLE I-2

### 3-Bromo-6,7,8,9,10,11-hexahydro-12H-cycloocta[b]thiochromen-12-one

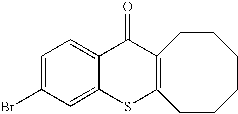

MS (EI+): 322 (M);

¹H-NMR (200 MHz, DMSO): δ=1.29-1.63 (m, 6H); 1.70-1.83 (m, 2H); 2.82-2.90 (m, 4H); 7.71-7.76 (dd, J=8.5 Hz, 2 Hz, 1H); 8.17 (d, J=2 Hz; 1H); 8.19-8.24 (d, J=8.5 Hz; 1H).

### EXAMPLE I-3

### 6-Bromo-3,3-dimnethyl-1,2,3,4-tetrahydro-9H-thioxanthen-9-one

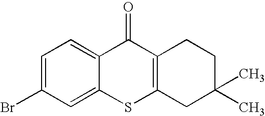

MS (EI+): 322 (M);

¹H-NMR (300 MHz, CDCl₃): δ=1.03 (s, 6H); 1.62 (t, J=6.5 Hz; 2H); 2.46 (s, 2H); 2.70 (m, 2H); 7.55-7.58 (dd, J=8.5, 2 Hz; 1H); 7.67 (d, J=2 Hz; 1H); 8.35 (d, J=8.5 Hz; 1H).

### EXAMPLE I-4

### 6-Bromo-3-spirobutyl-1,2,3,4-tetrahydro-9H-thioxanthen-9-one

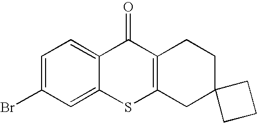

MS (EI+): 334 (M);

¹H-NMR (300 MHz, CDCl₃): δ=1.82-1.96 (m, 8H); 2.67-2.73 (m, 4H); 7.53-7.59 (dd, J=8.5, 2 Hz; 1H); 7.67 (d, J=2 Hz; 1H); 8.33 (d, J=8.5 Hz; 1H).

### EXAMPLE I-5

### 3-Ethyl-9-oxo-2,3,4,9-tetrahydro-1H-thioxanthene-6-carbonitrile

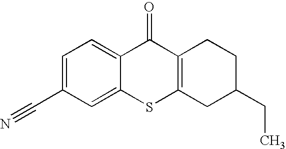

2.00 g (5.02 mmol) of 6-bromo-3-ethyl-1,2,3,4-tetrahydro-9H-thioxanthen-9-one (Example I-1), 0.62 g (5.27 mmol) of zinc cyanide and 0.58 g (0.50 mmol) of tetra-kis(triphenylphosphine)palladium(0) are dissolved in 20 ml of DMF under argon and heated at 80° C. overnight.

For working up, cooling to normal temperature is followed by 15 ml of 25% strength ammonia solution and 25 ml of water being added, and the precipitate being filtered off and washed with 75 ml of water. Drying in vacuo and recrystallization from cyclohexane/ethyl acetate (3:2) affords 1.28 g (95%) of the target compound.

MS (CI+): 287 (M+NH₄), 270 (M+H);

¹H-NMR (200 MHz, DMSO): δ=0.94 (t, J=7 Hz; 3H); 1.24-1.46 (m, 3H); 1.57-1.77 (m, 1H); 1.89-2.04 (m, 1H); 2.30-2.56 (m, 2H); 2.67-2.85 (m, 2H); 7.91-7.9 (dd, J=8.5 Hz, 1.5 Hz; 1H); 8.38-8.42 (d, J=8.5 Hz; 1H); 8.49 (d, J=1 Hz; 1H).

### EXAMPLE I-6

### 3-Ethyl-6-hydroxy-1,2,3,4-tetrahydro-9H-thioxanthen-9-one

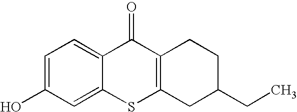

1.00 g (3.64 mmol) of 3-ethyl-6-methoxy-1,2,3,4-tetrahydro-9H-thioxanthen-9-one (Example 1-1) is introduced in 20 ml of acetic acid and, after addition of 8 ml of 48% strength hydrobromic acid, the mixture is stirred overnight and for a further three days at the reflux temperature until the precursor has completely reacted.

Working up is by substantially concentrating in a rotary evaporator, adding the residue to water and extracting the mixture three times with ethyl acetate. The combined organic phases are dried and concentrated. The crude product obtained in this way is absorbed onto silica gel and flash-chromatographed on about 150 g of silica gel, starting with cyclohexane and then with a cyclohexane/ethyl acetate 20:1 to 5:1 gradient. The product fractions are recrystallized from cyclohexane/ethyl acetate 5:1.

655 mg (69%) of the target compound are obtained in this way.

MS (CI+): 261 (M+H);

¹H-NMR (300 MHz, DMSO): δ=0.93 (t, J=7.5 Hz; 3H); 1.25-1.41 (m, 3H); 1.56-1.68 (m, 1H); 1.88-1.96 (m, 1H); 2.26-2.43 (m, 2H); 2.62-2.75 (m, 2H); 6.95 (m, 2H); 7.05-7.32 (m, b, 1H); 8.16-8.19 (m, 1H).

### EXAMPLE I-7

### 3-Ethyl-N′-hydroxy-9-oxo-2,3,4,9-tetrahydro-1H-thioxanthene-6-carboximidamide

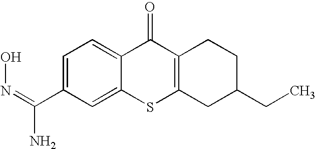

129 mg (1.86 mmol) of hydroxylammonium chloride are introduced into 1 ml of DMSO under argon, 0.26 ml (1.86 mmol) of triethylamine is added, and the mixture is stirred for about 5 min. The insoluble solid is filtered off and washed with tetrahydrofuran. The filtrate is freed of THF in a rotary evaporator and, after dropwise addition of 100 mg (0.37 mmol) of 3-ethyl-9-oxo-2,3,4,9-tetrahydro-1H-thioxanthene-6-carbonitrile (Example I-5) dissolved in DMSO to the remaining DMSO solution, the reaction mixture is stirred at 75° C. overnight.

For working up, the reaction mixture is diluted with 3 ml of DMSO/2 ml of acetonitrile and purified without further treatment by preparative HPLC. 85.3 mg (76%) of the target compound are obtained in this way.

MS (EI+): 302 (M);

¹H-NMR (200 MHz, DMSO): δ=0.95 (t, J=7 Hz; 3H); 1.24-1.46 (m, 3H); 1.56-1.76 (m, 1H); 1.88-2.03 (m, 1H); 2.28-2.43 (m, 2H); 2.71 (m, 1H); 2.80 (m, 1H); 6.04 (s, 2H); 7.84-7.89 (dd, J=8.5 Hz, 1.5 Hz, 1H); 7.99 (s, 1H); 8.26 (d, J=8.5 Hz; 1H); 10.03 (s, 1H).

The following were prepared in analogy to the method of Example I-7:

### EXAMPLE I-8

### N′-Hydroxy-12-oxo-6,8,9,10,11,12-hexahydro-7H-cycloocta[b]thiochromene-3-carboximidamide

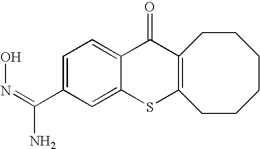

MS (EI+): 302 (M);

¹H-NMR (200 MHz, DMSO): δ=1.32-1.50 (m, 4H); 1.54-1.63 (m, 2H); 1.73-1.82 (m, 2H); 2.85-2.91 (m, 4H); 6.04 (s, 2H); 7.86-7.89 (dd, J=8.5 Hz, 1.5 Hz, 1H); 8.02 (d, J=1.5 Hz; 1H); 8.29 (d, J=8.5 Hz; 1H); 10.32 (s, 1H).

### EXAMPLE I-9

### N′-Hydroxy-3,3-dimethyl-9-oxo-2,3,4,9-tetrahydro-1H-thioxanthene-6-carboximidamide

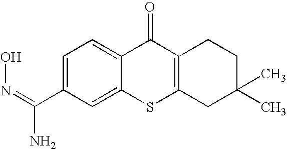

MS (CI+): 303 (M+H);

¹H-NMR (300 MHz, DMSO): δ=0.99 (s, 6H); 1.58 (t, J=6.5 Hz; 2H); 2.53-2.59 (m, 4H); 6.03 (s, 2H); 7.85-7.89 (dd, J=8.5 Hz, 1.5 Hz, 1H); 8.00 (d, J=1.5 Hz; 1H); 8.28 (d, J=8.5Hz; 1H); 10.03 (s, 1H).

### EXAMPLE I-10

### 6-Chloro-3-ethyl-7-fluoro-6-1,2,3,4-tetrahydro-9H-thioxanthen-9-one

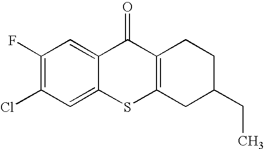

60 g of polyphosphoric acid are heated at 110° C. under argon for 10 min. After cooling, 5.00 g (29.8 mmol) of 3-chloro-4-fluorophenylthiol and 6.50 g (32.8 mmol) of ethyl 4-ethyl-2-oxo-cyclohexanecarboxylate are added, and the mixture is stirred at an oil bath temperature of 90° C. for 3.5 h. 200 g of ice and 200 ml of dichloromethane are added to the reaction mixture and, after thawing, the phases are separated. The aqueous phase is extracted twice more with dichloromethane, and the combined organic phases are washed twice with sodium hydroxide solution (1 N) and once with saturated aqueous sodium chloride solution. After drying over sodium sulfate and filtration, the solvent is removed by distillation under weak vacuum. The major part of the pure product is obtained by stirring the resulting solid in cyclohexane. Chromatography of the filtrate on silica gel (0.04-0.063 nm) with cyclohexane/ethyl acetate 40:1/30:1/20:1/10:1 as mobile phase affords a mixture which is subsequently purified by preparative HPLC.

Yield: 3.114 g (35.2% of theory).

R_{f} (cyclohexane/ethyl acetate 5:1)=0.65.

MS (EI): 297 (M+H).

HPLC, retention time=5.69 min (LC-MS).

¹H-NMR (200 MHz, CDCl₃): δ=0.99 (t, 3H), 1.32-1.51 (m, 3H), 1.59-1.82 (m, 1H), 1.97-2.13 (m, 1H), 2.32-2.60 (m, 2H), 2.62-2.80 (m, 1H), 2.81-2.99 (m, 1H), 7.58 (d, 1H), 8.24 (d, 1H).

### EXAMPLE I-11

### 3-Ethyl-9-oxo-2,3,4,9-tetrahydro-1H-thioxanthene-6-carboxylic acid

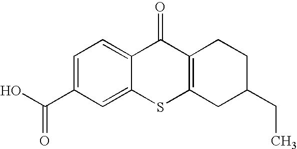

0.281 g (1.05 mmol) of 3-ethyl-9-oxo-2,3,4,9-tetrahydro-1H-thioxanthene-6-carbonitrile (Example I-5) is stirred in 5.5 ml of sulfuric acid (70% in water) at 120° C. for 2.5 h. After cooling, the solution is added to 2 ml of ice-water, and the resulting precipitate is initially filtered off and then taken up in sodium bicarbonate (10% strength aqueous solution). The carboxylic acid precipitates as a white solid after acidification with sulfuric acid (70% in water).

Yield: 262.8 mg (87.2% of theory).

R_{f} (CHCl₃:MeOH:AcOH:H₂O=120:30:3:3)=0.85.

MS (EI): 288 (M).

HPLC, retention time=4.64 min.

¹H-NMR (200 MHz, CDCl₃): δ=0.82 (t, 3H), 1.14-1.35 (m, 3H), 1.43-1.65 (m, 1 H), 1.78-1.95 (m, 1H), 2.18-2.44 (m, 2H), 2.50-2.81 (m, 2H), 7.87 (dd, 1H), 8.05 (d, 1H), 8.32 (d, 1H), 12.63 (br. s, 1H).

### EXAMPLE I-12

### Ethyl (4,4-diethyl-2-oxocyclohexyl)carboxylate

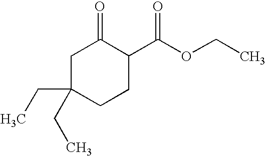

634 mg (4.62 mmol) of phosphorus trichloride are added dropwise to 2.1 g (12.5 mmol) of 5,5-diethylcyclohexane-1,3-dione (Kon, G. .A. R., Linstaed, R. P., J. Chem. Soc., (1925), 127, 819) under argon in chloroform. The mixture is heated to reflux for 3 h. It is concentrated, the residue is taken up in ice-cold water and extracted three times with ether, and the combined organic phases are dried and again concentrated in vacuo.

The resulting crude product is dissolved in 50 ml of anhydrous tetrahydrofuran, mixed with 1 g of palladium on carbon (5% palladium) and hydrogenated under atmospheric pressure overnight. After working up by filtration and concentration, because conversion is incomplete the hydrogenation is repeated with 1.6 g of Pd/C (5% Pd) in 50 ml of anhydrous tetrahydrofaran under atmosphenc pressure overnight.

This procedure is repeated once more.

1.90 g (98%) of 3,3-diethylcyclohexanone are obtained in this way, acording to GC-MS as 97% pure product, whch is reacted further as such.

985 mg (about 25 mmol) of sodium hydride (60%) are introduced into argon into a reaction flask, the oil is removed with petroleum ether, and 15 ml of diethyl carbonate are added. Then one drop of ethanol is added to the resulting suspension, and the 1.90 g (12.3 mmol) of 3,3-diethylcyclohexanone obtained above, dissolved in about 10 ml of diethylcarbonate, are slowly added dropwise. The mixture is stirred at normal temperature overnight, during which slow evolution of hydrogen is observable.

For working up, 30 g of ice and about 100 ml of water are slowly added to the reaction mixture, and then the pH is adjusted to 5 with about 6 ml of concentrated acetic acid.

The resulting solution is extracted three times with diethyl ether. The combined organic phases are washed twice with cold saturated sodium bicarbonate solution and once with saturated brine and dried over magnesium sulfate. The solution is then concentrated to about 5 ml in vacuo in a rotary evaporator.

Kugelrohr distillation affords 1.37 g (49%) of the target compound in sufficient purity for the next stages.

¹H-NMR (200 MHz, CDCl₃): the compound is substantially enolized: δ=0.7-0.95 (m, 6H), 1.13-1.5 (m, 9H), 2.04 (s, broad, 2H), 2.1-2.3 (m, 2H), 4.12-4.28 (m, 2H), 12.20 (s, broad, 1H).

### EXAMPLE I-13

### Ethyl (6-oxo-spiro[3.5]non-7-yl)-carboxylate

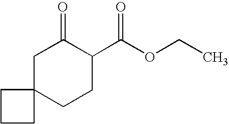

5.00 g (36.7 mmol) of spiro[3.5]non-7-en-6-one (Paulsen, H. et al., Angew. Chem., Int. Ed. (1999), 38, 3373) are introduced into 100 ml of ethylene glycol dimethyl 5 ether, and about 300 mg of Pd/C (5% Pd) are added. Hydrogenation is carried out under atmospheric pressure overnight.

For working up, the catalyst is removed by filtration through kieselguhr, and the filtrate is concentrated in vacuo.

5.21 g (91% pure according to GC analysis, equivalent to 93% yield of product) of spiro[3.5]nonan-6-one are obtained in this way.

One third of 5.00 g (36.2 mmol) of spiro[3.5]nonan-6-one dissolved in diethyl carbonate is added dropwise to a suspension of 2.89 g (60%, approx. 72 nunol) of sodium hydride in diethyl carbonate under argon. Only after addition of a little THF/pentane to activate the sodium hydride does a gentle but continuous evolution of hydrogen start. The remainder of the ketone is slowly added in portions distributed over the day. The mixture is then stirred at normal temperature overnight.

For working up, 20 g of ice and about 100 ml of water are slowly added to the reaction mixture. There is initial formation of a viscous sludge which slowly dissolves over time. A pH of 5 is adjusted with approx. 10 ml of concentrated acetic acid.

The resulting solution is extracted three times with diethyl ether. The combined organic phases are washed twice with cold saturated sodium bicarbonate solution and once with saturated brine and dried over magnesium sulfate. After concentration in a rotary evaporator, about 70 ml of liquid remains.

For purification, initially excess diethyl carbonate is distilled out by distillation at 50-60° C. and 70-80 mbar.

The residue is subjected to short-path distillation with Vigreux attachment at about 0.25 mbar and 60-70° C.

3.71 g (49%) of the target compound are obtained in sufficient purity for the following stages in this way.

¹H-NMR (200 MHz, DMSO-d₆): the compound is substantially enolized: δ=1.11-1.29 (m, 3H), 1.50-2.05 (m, 8H), 2.17 (t, broad, J=6 Hz, 2H), 2.31 (s, broad, 2H), 4.0-4.25 (m, 2H), 12.11 (s, broad, 1H).

### EXAMPLE I-14

### Ethyl (2-oxo-spiro[5.5]undec-3-yl)carboxylate

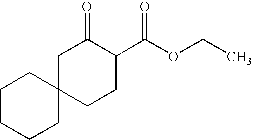

Under argon, 1.11 g (27.7 mmol) of sodium hydride (60%) are introduced into a flask, the oil is removed with petroleum ether and 15 ml of diethyl carbonate are added. One drop of ethanol is added to the resulting suspension, and 2.30 g (13.8 mmol) of spiro[5.5]undecan-2-one (De Jongh, H. A. P. and Wynberg, K., Tetrahedron (1964), 20, 2553) dissolved in about 10 ml of diethyl carbonate are slowly added dropwise. The mixture is stirred at normal temperature overnight, during which slow evolution of hydrogen is observable.

For working up, 30 g of ice and about 100 ml of water are slowly added to the reaction mixture, and then the pH is adjusted to 5 with about 6 ml of concentrated acetic acid.

The resulting solution is extracted three times with diethyl ether. The combined organic phases are washed twice with cold saturated sodium bicarbonate solution and once with saturated brine and dried over magnesium sulfate. This is followed by evaporation to about 5 ml in vacuo in a rotary evaporator.

Kugelrohr distillation affords 2.43 g (74%) of the target compound.

¹H-NMR (200 MHz, CDCl₃): the compound is substantially enolized: δ=1.2-1.6 (m, 15H), 2.11 (s, broad, 2H), 2.13-2.28 (m, 2H), 4.11-4.29 (m, 2H), 12.19 (s, broad, 1H).

The following are prepared in analogy to the method of Example I-1:

### EXAMPLE I-15

### 6-Bromo-3,3-diethyl-1,2,3,4-tetrahydro-9H-thioxanthen-9-one

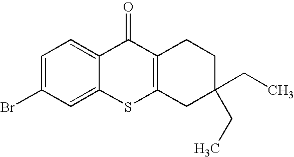

MS (EI+): 350 (M);

¹H-NMR (300 MHz, CDCl₃): δ=0.85 (t, 6H); 1.37 (mc, 4H); 1.64 (t, 2H); 2.45 (s, br, 2H); 2.65 (t, br, 2H); 7.56 (dd, 1H); 7.67 (d, 1H); 8.35 (d, 1H).

### EXAMPLE I-16

### 6-Bromo-3-spirohexyl-1,2,3,4-tetrahydro-9H-thioxanthen-9-one

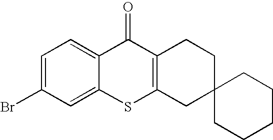

MS (CI+): 363 (M+H);

¹H-NMR (300 MHz, CDCl₃): δ=1.3-1.6 (m, 10H); 1.70 (t, 2H); 2.51 (s, br, 2H); 2.66 (t, br, 2H); 7.56 (dd, 1H); 7.66 (d, 1H); 8.34 (d, 1H).

The following are prepared in analogy to the method of Example I-5:

### EXAMPLE I-17

### 3,3-Dimethyl-9-oxo-2,3,4,9-tetrahydro-1H-thioxanthene-6-carbonitrile

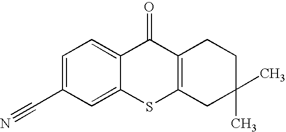

MS (CI+): 270 (M+H);

¹H-NMR (300 MHz, DMSO): δ=0.99 (s, 6H); 1.58 (t, 2H); 2.45-2.6 (m, 4H); 7.94 (dd, 1H); 8.42 (d, 1H); 8.50 (d, 1H).

### EXAMPLE I-18

### 3,3-Diethyl-9-oxo-2,3,4,9-tetrahydro-1H-thioxanthene-6-carbonitrile

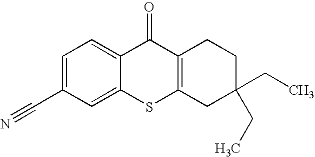

MS (CI+): 298 (M+H);

¹H-NMR (300 MHz, CDCl₃): δ=0.86 (t, 6H); 1.38 (mc, 4H); 1.66 (t, 2H); 2.49 (s, br, 2H); 2.67 (t, br, 2H); 7.67 (dd, 1H); 7.83 (d, 1H); 8.58 (d, 1H).

### EXAMPLE I-19

### 9-Oxo-3-spirobutyl-2,3,4,9-tetrahydro-1-thioxanthene-6-carbonitrile

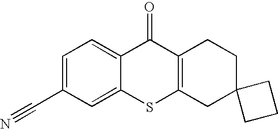

MS (CI+): 282 (M+H);

¹H-NMR (300 MHz, CDCl₃): δ=1.80-2.05 (m, 8H); 2.72 (tt, 2H); 2.76 (s, br, 2H); 7.68 (dd, 1H); 7.84 (d, 1H); 8.57 (d, 1H).

### EXAMPLE I-20

### 9-Oxo-3-spirohexyl-2,3,4,9-tetrahydro-1H-thioxanthene-6-carbonitrile

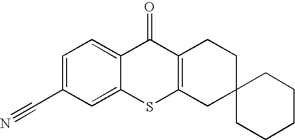

MS (EI+): 309 (M);

¹H-NMR:(300 MHz, CDCl₃): δ=1.35-1.6 (m, 10H); 1.72 (t, 2H); 2.55 (s, br, 2H); 2.69 (t, br, 2H); 7.67 (dd, 1H); 7.83 (d, 1H); 8.58 (d, 1H).

### EXAMPLE I-21

### 12-Oxo-6,7,8,9,10,11-hexahydro-12H-cycloocta[b]thiochromene-3-carbonitrile

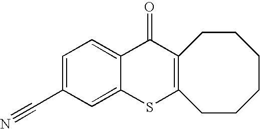

The product is purified by crystallization from acetone.

MS (CI+): 270 (M+H);

¹H-NMR (300 MHz, DMSO): δ=1.30-1.52 (m, 4H); 1.53-1.65 (m, 2H); 1.70-1.85 (m, 2H); 2.90 (m, 4H); 7.94 (dd, 1H); 8.43 (d, 1H); 8.50 (d, 1H).

The following are prepared in analogy to the method of Example I-11:

### EXAMPLE I-22

### 3,3-Dimethyl-9-oxo-2,3,4,9-tetrahydro-1H-thioxanthene-6-carboxylic acid

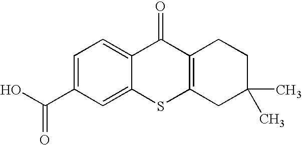

MS (ESI+): 289 (M+H);

HPLC: Rₜ=4.57 min;

¹H-NMR (200 MHz, DMSO): δ=0.99 (s, 6H); 1.58 (t, 2H); 2.45-2.65 (m, 4H); 8.02 (dd, 1H); 8.29 (d, 1H); 8.41 (d, 1H); 13.58 (s, br, 1H).

### EXAMPLE I-23

### 12-Oxo-6,7,8,9,10,11-hexahydro-12H-cycloocta[b]thiochromene-3-carboxylic acid

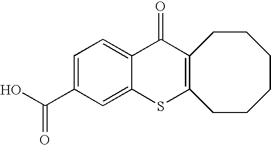

98 mg (1.48 mmol) of potassium hydroxide powder are added under an argon atmosphere to 200 mg (0.74 mmol) of 12-oxo-6,7,8,9,10,11-hexahydro-12H-cycloocta[b]thiochromene-3-carbonitrile in 10 ml of 1,2-ethanediol, and the mixture is stirred at 120° C. overnight. After cooling, a little water is added, and the mixture is then acidified to pH 2 with 1N hydrochloric acid. The precipitate is filtered off and washed with water.

160 mg (75%) of the target compound are obtained in this way.

HPLC: Rₜ=4.38 min;

¹H-NMR (300 MHz, THF): δ=1.40-1.60 (m, 4H); 1.68 (mc, 2H); 1.70-1.85 (mc, 2H); 2.92 (mc, 4H); 8.04 (dd, 1H); 8.26 (d, 1H); 8.48 (d, 1H); 11-13 (br, 1H).

### EXAMPLE I-24

### 6-(Cyanomethyl)-3,3-dimethyl-1,2,3,4-tetrahydro-9H-thioxanthen-9-one

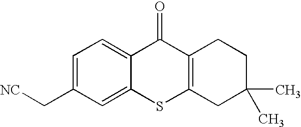

300 mg (1.02 mmol) of 6-(chloromethyl)-3,3-dimethyl-1,2,3,4-tetrahydro-9H-thioxanthen-9-one, 80.1 mg (1.23 mmol) of potassium cyanide and 13.5 mg (0.05 mmol) of 18-crown-6 are mixed in 1.00 ml of N,N-dimethylformamide under argon and then stirred at a bath temperature of 40° C. overnight. For working up, the mixture is dried with magnesium sulfate. The solvent is concentrated in vacuo, and the residue is chromatographed (preparative HPLC, acetonitrile/water 50:50-95:5). 105 mg (36%) of the target compound are obtained in this way.

MS (ESI+): 284 (M+H);

HPLC: Rₜ=4.76 min;

¹H-NMR (300 MHz, DMSO): δ=0.99 (s, 6H); 1.58 (t, 2H); 2.45-2.65 (m, 4H); 4.21 (s, 2H); 7.53 (d, 1H); 7.73 (s, 1H); 8.34 (d, 1H).

### EXAMPLE I-25

### (3,3-Dimethyl-9-oxo-2,3,4,9-tetrahydro-1H-thioxanthen-6-yl)acetic acid

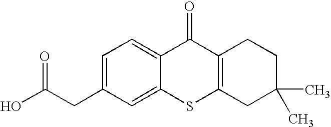

109 mg (0.38 mmol) of 6-(cyanomethyl)-3,3-dimethyl-1,2,3,4-tetrahydro-9H-thioxanthen-9-one are suspended in 70% strength sulfuric acid and stirred at a bath temperature of 120° C. under argon. After the reaction is complete (about 3 hours), the mixture is allowed to cool and is added to ice-water. The precipitated solid is filtered off with suction, washed with water and dried in vacuo. 109 mg (93%) of the target compound are obtained in this way.

MS (ESI+): 303 (M+H);

HPLC: Rₜ=4.47 min;

¹H-NMR (200 MHz, DMSO): δ=0.99 (s, 6H); 1.57 (t, 2H); 2.4-2.65 (m, 4H); 3.74 (s, 2H); 7.46 (dd, 1H); 7.65 (s, br, 1H); 8.26 (d, 1H); 12.1-12.9 (s, br, 1H).

### EXEMPLARY EMBODIMENTS 1

The following were prepared in analogy to the method of Example I-1:

### EXAMPLE 1-1

### 3-Ethyl-6-methoxy-1,2,3,4-tetrahydro-9H-thioxanthen-9-one

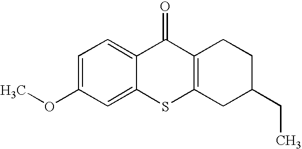

MS (EI+): 274 (M);

¹H-NMR (400 MHz, CDCl₃): δ=0.99 (t, J=7.5Hz; 3H); 1.34-1.44 (m, 3H); 1.64-1.72 (m, 1H); 1.97-2.04 (m, 1H); 2.33-2.40 (m, 1H); 2.44-2.54 (m, 1H); 2.69 (m, 1H); 2.86-2.93 (m, 1H); 3.88 (s, 3H); 6.88 (d, J=2.5; 1H); 6.99-7.03 (dd, J=9 Hz, 2.5 Hz; 1H); 8.41 (d, J=9 Hz; 1H).

### EXAMPLE 1-2

### 3-Ethyl-6-methyl-1,2,3,4-tetrahydro-9H-thioxanthen-9-one

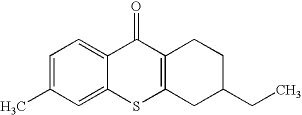

MS (EI+): 258 (M);

¹H-NMR (300 MHz, CDCl₃): δ=0.99 (t, J=7.5Hz; 3H); 1.33-1.47 (m, 3H); 1.64-1.76 (m, 1H); 1.98-2.07 (m, 1H); 2.34-2.42 (m, 1H); 2.44 (s, 3H); 2.46-2.56 (m, 1H); 2.66-2.74 (m, 1H); 2.86-2.96 (m, 1H); 7.25-7.29 (m, 2H); 8.38 (d, J=9 Hz; 1H).

### EXAMPLE 1-3

### 3-Ethyl-6-(1H-tetrazol-5-yl)-1,2,3,4-tetrahydro-9H-thioxanthen-9-one

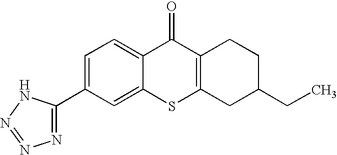

0.50 g (1.86 mmol) of 3-ethyl-9-oxo-2,3,4,9-tetrahydro-1H-thioxanthene-6-carbonitrile (Example I-5), 0.51 g (3.71 mmol) of triethylamrnonium hydrochloride and 0.24 g (3.71 mmol) of sodium azide are heated in toluene to 100° C. and stirred at this temperature overnight.

After a TLC check for completeness of conversion, the reaction mixture is allowed to cool and added to 100 ml of water/20 ml of toluene, stirred vigorously for 10 min, acidified to about pH 3 with 5N hydrochloric acid and stirred for a further 10 min, and the precipitated solid is filtered off with suction and washed twice with water. The remaining crude product is dried and recrystallized from cyclohexane/ethyl acetate approx. 1:40. 290 mg (50%) of the target compound are obtained in this way.

MS (CI+): 313 (M+H);

¹H-NMR (300 MHz, DMSO): δ=0.95 (t, J=7.5 Hz; 3H); 1.30-1.43 (m, 3H); 1.60-1.74 (m, 1H); 1.92-2.02 (m, 1H); 2.33-2.47 (m, 2H); 2.72-2.82 (m, 2H); 3.00-3.60 (m, b, 1H); 8.14-8.18 (dd, J=8.5 Hz, 1.5 Hz; 1H); 8.39 (d, J=1.5 Hz; 1H), 8.47-8.50 (d, J=8.5 Hz; 1H).

The following were prepared in analogy to the method of Example 1-3:

### EXAMPLE 1-4

### 3-(1H-Tetraazol-5-yl)-6,7,8,9,10,11-hexahydro-12H-cycloocta[b]thiochromen-12-one

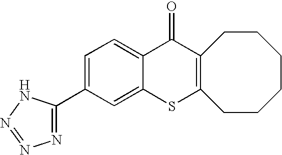

MS (EI+): 312 (M);

¹H-NMR (200 MHz, DMSO): δ=1.30-1.43 (m, 4H); 1.54-1.66 (m, 2H); 1.73-1.84 (m, 2H); 2.83-2.94 (m, 4H); 3.12-3.68 (m, b, 1H); 8.15-8.20 (dd, J=8.5 Hz, 1.5 Hz; 1H); 8.43 (s, 1H), 8.49-8.53 (d, J=8.5 Hz; 1H).

### EXAMPLE 1-5

### 3,3-Dimethyl-6-(1H-tetrazol-5-yl)-1,2,3,4-tetrahydro-9H-thioxanthen-9-one

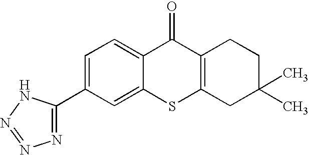

MS (CI+): 313 (M+H);

¹H-NMR (200 MHz, DMSO): δ=1.00 (s, 6H); 1.59 (t, J=6.5 Hz; 2H); 2.47-2.62 (m, 4H); 3.08-3.52 (s, b, 1H); 8.15-8.20 (dd, J=8.5 Hz, 1.5 Hz, 1H); 8.40 (s, 1H); 8.50 (d, J=8.5 Hz; 1H).

### EXAMPLE 1-6

### 3-Spirobutyl-6-(1H-tetrazol-5-yl)-1,2,3,4-tetrahydro-9H-thioxanthen-9-one

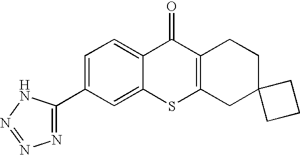

MS (ESI): 325 (M+H);

¹H-NMR (200 MHz, DMSO): δ=1.79-1.99 (m, 8H); 2.56-2.63 (t, J=6 Hz;2H); 2.84 (s, 2H); 3.12-3.60 (s, b, 1H); 8.14-8.19 (dd, J=8.5 Hz, 1.5 Hz; 1H); 8.41 (d, J=1.5 Hz; 1H); 8.47-8.51 (d, J=8.5 Hz; 1H).

### EXAMPLE 1-7

### 3-Spirohexyl-6-(1H-tetrazol-5-yl)-1,2,3,4-tetrahydro-9H-thioxanthen-9-one

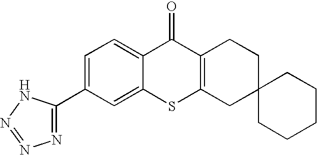

MS (EI+): 352 (M);

¹H-NMR (300 MHz, DMSO): δ=1.33-1.48 (m, 10H); 1.67 (t, J=6.5 Hz; 2H); 2.56 (t, J=6.5 Hz; 2H); 2.61 (s, 2H); 3.17-3.45 (s, b, 1H); 8.15-8.19 (dd, J=8.5 Hz, 1H); 8.40 (d, J=1.5 Hz; 1H); 8.48-8.51 (d, J=8.5 Hz; 1H).

### EXAMPLE 1-8

### 3-Ethyl-6-[3-(1H-tetrazol-5-yl)phenyl]-1,2,3,4-tetrahydro-9H-thioxanthen-9-one

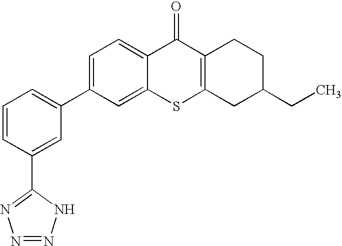

MS (ESI): 389 (M+H);

¹H-NMR (300 MHz, DMSO): δ=0.96 (t, J=7.5 Hz; 3H); 1.31-1.44 (m, 3H); 1.62-1.74 (m, 1H); 1.93-2.02 (m, 1H); 2.34-2.53 (m, 2H); 2.72-2.82 (m, 2H); 3.23-3.40 (s, b, 1H); 7.73-7.79 (t, J=8 Hz; 1H); 7.95-7.98 (dd, J=8.5 Hz, 1.5 Hz; 1H); 8.05 (d, J=8 Hz; 1H); 8.12 (d, J=8 Hz; 1H); 8.19. (d, J=1.5 Hz; 1H); 8.42-8.46 (m, 2H).

### EXAMPLE 1-9

### 3-Ethyl-6-(2-methyl-2H-tetrazol-5-yl)-1,2,3,4-tetrahydro-9H-thioxanthen-9-one

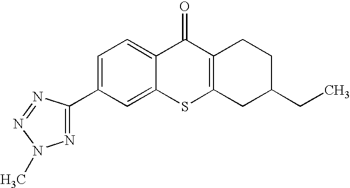

Under argon, 91.0 mg (0.29 mmol) of 3-ethyl-6-(1H-tetrazol-5-yl)-1,2,3,4-tetrahydro-9H-thioxanthen-9-one (Example 1-3) are dissolved in 5 ml of DMF, and 95 mg (0.29 mmol) of cesium carbonate are added. The mixture is heated at 60° C. for 1 h, cooled again to nonmal temperature and, after addition of 0.03 ml (0.45 mmol) of iodomethane, left to stir at normal temperature overnight. For working up, about 10 ml of water and ethyl acetate are added to the mixture, which is stirred for 5 min and added to 50 ml of water. Three more extractions with ethyl acetate are carried out, and the combined organic phases are dried and concentrated in a rotary evaporator.

The crude product obtained in this way is fractionated and purified by preparative HPLC.

54.9 mg (58%) of the target compound are obtained in this way.

MS (EI+): 326 (M);

¹H-NMR (300 MHz, CDCl₃): δ=1.00 (t, J=7.5 Hz; 3H); 1.38-1.49 (m, 3H); 1.68-1.79 (m, 1H); 2.01-2.10 (m, 1H); 2.40-2.60 (m, 2H); 2.72-2.79 (m, 1H); 2.91-2.97 (m, 1H); 4.44 (s, 3H); 8.17-8.21 (dd, J=8.5, 1.5 Hz; 1H); 8.33 (d, J=1.5 Hz; 1H); 8.60 (d, J=8.5 Hz; 1H).

The byproduct isolated are:

5.2 mg (5.5%) of 3-ethyl-6-(1-methyl-1H-tetrazol-5-yl)-1,2,3,4-tetrahydro-9H-thioxanthen-9-one

¹H-NMR (400 MHz, CDCl₃): δ=1.01 (t, J=7.5Hz; 3H); 1.38-1.49 (m, 3H); 1.70-1.79 (m, 1H); 2.03-2.11 (m, 1H); 2.42-2.61 (m, 2H); 2.73-2.80 (m, 1H); 2.91-2.97 (m, 1H); 4.25 (s, 3H); 7.75-7.77 (dd, J=8.5, 1.5 Hz; 1H); 8.01 (d, J=1.5 Hz; 1H); 8.67 (d, J=8.5 Hz; 1H).

### EXAMPLE 1-10

### 3-Ethyl-6-(2-thienyl)-1,2,3,4-tetrahydro-9H-thioxanthen-9-one

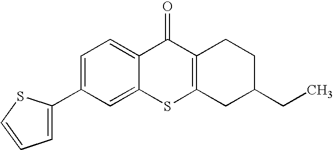

100 mg (0.31 mmol) of 6-bromo-3-ethyl-1,2,3,4-tetrahydro-9H-thioxanthen-9-one (Example I-1) are introduced into 2.0 ml of 1,2-dimethoxyethane and, after addition of 47.5 mg (0.37 mmol) of thiophene-2-boronic acid, 0.34 ml (0.68 mmol) of 2M aqueous sodium carbonate solution and 10 mg of dichlorobis(triphenylphosphine)palladium(II), heated to reflux for about 2 h until the reaction is complete.

After cooling, the reaction mixture is filtered through a cartridge with 1 g of silica gel, and the eluate is concentrated and separated by preparative HPLC.

Drying of the product fractions in vacuo results in 76.8 mg (76%) of the target compound.

MS (CI+): 327 (M+H);

¹H-NMR (200 MHz, CDCl₃): δ=1.00 (t, J=7.5 Hz; 3H); 1.35-1.51 (m, 3H); 1.62-1.81 (m, 1H); 1.98-2.11 (m, 1H); 2.34-2.61 (m, 2H); 2.66-2.79 (m, 1H); 2.84-3.00 (m, 1H); 7.11-7.16 (dd, J=5 Hz, 3.5 Hz; 1H); 7.38-7.41 (dd, J=5 Hz, 1 Hz; 1H); 7.45-7.47 (dd, J=3.5Hz, 1Hz; 1H); 7.67-7.72 (m, 2H); 8.47-8.51 (m, 1H).

The following were prepared in analogy to the method of Example 1-10:

### EXAMPLE 1-11

### 3-(2-Thienyl)-6,7,8,9,10,11-hexahydro-12H-cycloocta[b]thiochromen-12-one

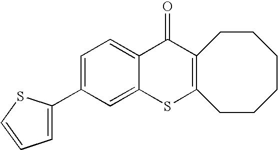

MS (CI+): 327 (M+H);

¹H-NMR (300 MHz, CDCl₃): δ=1.40-1.55 (m, 4H); 1.69-1.77 (m, 2H); 1.81-1.89 (m, 2H); 2.84-2.88 (m, 2H); 2.93-2.97 (m, 2H); 7.12-7.15 (dd, J=5 Hz, 4 Hz; 1H); 7.39-7.40 (dd, J=5 Hz, 1Hz; 1H); 7.46-7.47 (dd, J=3.5Hz, 1Hz; 1H); 7.70-7.74 (m, 2H); 8.49-8.52 (m, 1H).

### EXAMPLE 1-12

### 3,3-Dimethyl-6-(2-thienyl)-1,2,3,4-tetrahydro-9H-thioxanthen-9-one

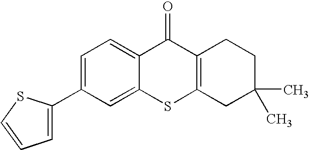

MS (EI+): 326 (M);

¹H-NMR (200 MHz, CDCl₃): δ=1.05 (s, 6H); 1.64 (t, J=6.5 Hz; 2H); 2.48 (s, 2H); 2.73 (t, J=6.5 Hz; 2H); 7.11-7.16 (dd, J=5 Hz, 3.5Hz; 1H); 7.38-7.41 (dd, J=5 Hz, 1 Hz; 1H); 7.45-7.48 (dd, J=3.5 Hz, 1Hz; 1H); 7.68-7.74 (m, 2H); 8.48-8.52 (m, 1H).

### EXAMPLE 1-13

### 3-Spirobutyl-6-(2-thienyl)-1,2,3,4-tetrahydro-9H-thioxanthen-9-one

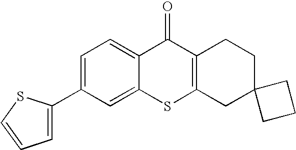

MS (CI+): 339 (M+H);

¹H-NMR(300 MHz, CDCl₃): δ=1.80-2.00 (m, 8H); 2.68-2.81 (m, 4H); 7.12-7.15 (dd, J=5 Hz, 3.5 Hz; 1H); 7.38-7.40 (dd, J=5 Hz, 1 Hz; 1H); 7.45-7.47 (m, 1H); 7.68-7.71 (m, 2H); 8.47-8.50 (m, 1H).

### EXAMPLE 1-14

### 3-Ethyl-6-(4-methyl-2-thienyl)-1,2,3,4-tetrahydro-9H-thioxanthen-9-one

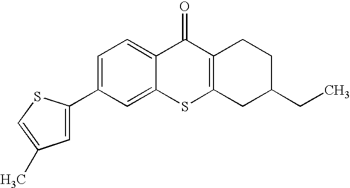

MS (CI+): 341 (M+H);

¹H-NMR (200 MHz, CDCl₃): δ=1.00 (t, J=7.5 Hz; 3H); 1.34-1.50 (m, 3H); 1.61-1.79 (m, 1H); 1.98-2.12 (m, 1H); 2.31 (s, 3H); 2.38-2.63 (m, 2H); 2.65-2.78 (m, 1H); 2.85-2.99 (m, 1H); 6.97 (s, 1H); 7.28 (m, 1H); 7.64-7.69 (m, 2H); 8.45-8.49 (m, 1H).

### EXAMPLE 1-15

### 6-(5-Acetyl-2-thienyl)-3-ethyl-1,2,3,4-tetrahydro-9H-thioxanthen-9-one

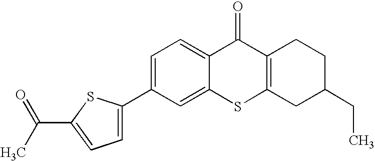

MS (ESI): 369 (M+H);

¹H-NMR (300 MHz, CDCl₃): δ=1.00 (t, J=7.5 Hz; 3H); 1.35-1.49 (m, 3H); 1.67-1.77 (m, 1H); 2.01-2.10 (m, 1H); 2.37-2.54 (m, 2H); 2.59 (s, 3H); 2.69-2.78 (m, 1H); 2.88-2.97 (m, 1H); 7.45 (d, J=4 Hz; 1H); 7.69-7.75 (m, 3H); 8.52 (d, J=8.5 Hz; 1H).

### EXAMPLE 1-16

### 3-Ethyl-6-(3-thienyl)-1,2,3,4-tetrahydro-9H-thioxanthen-9-one

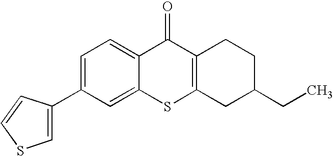

MS (CI+): 327 (M+H);

¹H-NMR (300 MHz, CDC₁₃): δ=1.00 (t, J=7.5Hz; 3H); 1.37-1.48 (m, 3H); 1.66-1.78 (m, 1H); 2.00-2.09 (m, 1H); 2.37-2.58 (m, 2H); 2.67-2.76 (m, 1H); 2.88-2.98 (m, 1H); 7.42-7.47 (m, 2H); 7.62 (m, 1H); 7.68-7.71 (m, 2H); 8.51 (m, 1H).

### EXAMPLE 1-17

### 3-Ethyl-6-phenyl-1,2,3,4-tetrahydro-9H-thioxanthen-9-one

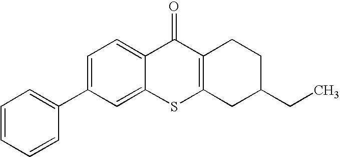

MS (CI+): 321 (M+H);

¹H-NMR (200 MHz, CDCl₃): δ=1.00 (t, J=7.5 Hz; 3H); 1.33-1.51 (m, 3H); 1.62-1.80 (m, 1H); 1.99-2.12 (m, 1H); 2.35-2.64 (m, 2H); 2.68-2.79 (m, 1H); 2.87-3.02 (m, 1H); 7.41-7.53 (m, 3H); 7.64-7.73 (m, 4H); 8.53-8.58 (m, 1H).

### EXAMPLE 1-18

### 6-Phenyl-3-spirobutyl-1,2,3,4-tetrahydro-9H-thioxanthen-9-one

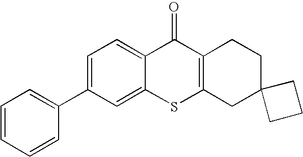

MS (CI+): 333 (M+H);

¹H-NMR (300 MHz, CDCl₃): δ=1.80-2.04 (m, 8H); 2.72-2.76 (m, 4H); 7.39-7.52 (m, 3H); 7.63-7.71 (m, 4H); 8.54-8.57 (m, 1H).

### EXAMPLE 1-19

### 3-Ethyl-6-(4-methylphenyl)-1,2,3,4-tetrahydro-9H-thioxanthen-9-one

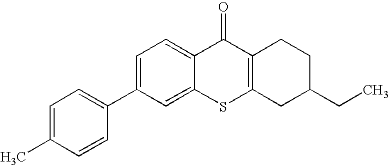

MS (CI+): 335 (M+H);

¹H-NMR (300 MHz, CDCl₃): δ=1.00 (t, J=7.5 Hz; 3H); 1.35-1.48 (m, 3H); 1.66-1.78 (m, 1H); 2.00-2.09 (m, 1H); 2.42 (s, 3H); 2.43-2.59 (m, 2H); 2.68-2.77 (m, 1H); 2.88-2.98 (m, 1H); 7.28-7.30 (d, J=8 Hz; 2H); 7.54-7.57 (d, J=8 Hz; 2H); 7.67-7.70 (m, 2H); 8.52-8.55 (m, 1H).

### EXAMPLE 1-20

### N-[3-(3-Ethyl-9-oxo-2,3,4,9-tetrahydro-1H-thioxanthen-6-yl)phenyl]acetamide

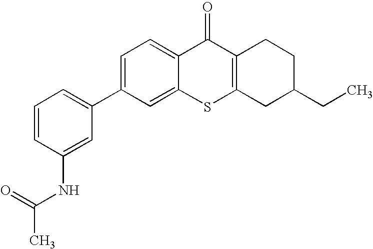

MS (CI+): 378 (M+H);

¹H-NMR (300 MHz, CDCl₃): δ=1.00 (t, J=7.5 Hz; 3H); 1.36-1.48 (m, 3H); 1.66-1.77 (m, 1H); 2.00-2.09 (m, 1H); 2.24 (s, 3H); 2.37-2.58 (m, 2H); 2.68-2.76 (m, 1H); 2.88-2.98 (m, 1H); 7.36-7.58 (m, 4H); 7.64-7.69 (m, 2H); 7.84 (s, 1H); 8.53 (m, 1H).

### EXAMPLE 1-21

### 3-Ethyl-6-(4-methoxyphenyl)-1,2,3,4-tetrahydro-9H-thioxanthen-9-one

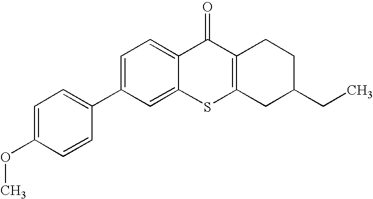

MS (CI+): 351 (M+H);

¹H-NMR (200 MHz, CDCl₃): δ=1.00 (t, J=7.5 Hz; 3H); 1.35-1.51 (m, 3H); 1.62-1.79 (m, 1H); 1.98-2.11 (m, 1H); 2.34-2.62 (m, 2H); 2.67-2.79 (m, 1H); 2.86-3.01 (m, 1H); 3.87 (s, 3H); 6.99-7.03 (m, 2H); 7.57-7.69 (m, 4H); 8.50-8.55 (m, 1H).

### EXAMPLE 1-22

### 6-(4-Methoxyphenyl)-3-spirobutyl-1,2,3,4-tetrahydro-9H-thioxanthen-9-one

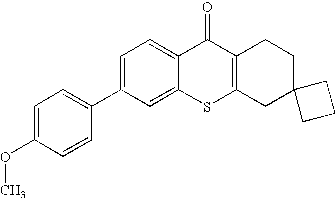

MS (CI+): 363 (M+H);

¹H-NMR (300 MHz, CDCl₃): δ=1.80-2.03 (m, 8H); 2.71-2.76 (m, 4H); 3.87 (s, 3H); 7.00-7.03 (m, 2H); 7.58-7.68 (m, 4H); 8.50-8.53 (m, 1H).

### EXAMPLE 1-23

### 3-Ethyl-6-(3-nitrophenyl)-1,2,3,4-tetrahydro-9H-thioxanthen-9-one-12-one

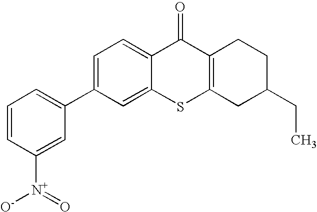

MS (CI+): 366 (M+H);

¹H-NMR (300 MHz, CDCl₃): δ=1.01 (t, J=7.5 Hz; 3H); 1.37-1.50 (m, 3H); 1.68-1.79 (m, 1H); 2.02-2.11 (m, 1H); 2.40-2.61 (m, 2H); 2.71-2.79 (m, 1H); 2.89-2.99 (m, 1H); 7.65-7.75 (m, 3H); 7.97-8.01 (m, 1H); 8.27-8.30 (m, 1H); 8.52 (m, 1H); 8.60-8.63 (m, 1H).

### EXAMPLE 1-24

### 6-(4-Chlorophenyl)-3-ethyl-1,2,3,4-tetrahydro-9H-thioxanthen-9-one

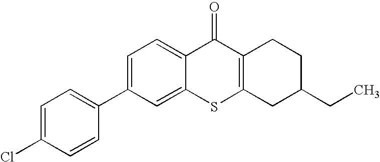

MS (CI+): 355 (M+H);

¹H-NMR (300 MHz, CDCl₃): δ=1.00 (t, J=7.5 Hz; 3H); 1.37-1.49 (m, 3H); 1.66-1.78 (m, 1H); 2.01-2.10 (m, 1H); 2.38-2.59 (m, 2H); 2.69-2.77 (m, 1H); 2.89-2.98 (m, 1H); 7.43-7.48 (m, 2H); 7.56-7.61 (m, 2H); 7.63-7.67 (m, 2H); 8.54-8.57 (m, 1H).

### EXAMPLE 1-25

### 3-Ethyl-6-[4-(hydroxymethyl)phenyl]-1,2,3,4-tetrahydro-9H-thioxanthen-9-one

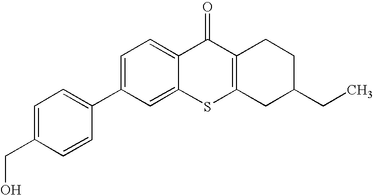

MS (CI+): 351 (M+H);

¹H-NMR (300 MHz, CDCl₃): δ=0.95 (t, J=7.5 Hz; 3H); 1.28-1.43 (m, 3H); 1.60-1.72 (m, 1H); 1.92-2.01 (m, 1H); 2.32-2.47 (m, 2H); 2.70-2.78 (m, 2H); 4.57 (d, J=5.5 Hz; 2H); 5.29 (t, J=5.5 Hz; 1H); 7.46 (d, J=8 Hz; 2H); 7.80 (d, J=8.5 Hz; 2H); 7.85-7.89 (dd, J=8.5 Hz, 1.5 Hz; 1H); 8.07 (d, J=1.5 Hz; 1H); 8.37 (m, 1H).

### EXAMPLE 1-26

### 4-(3-Ethyl-9-oxo-2,3,4,9-tetrahydro-1-thioxanthen-6-yl)benzonitrile

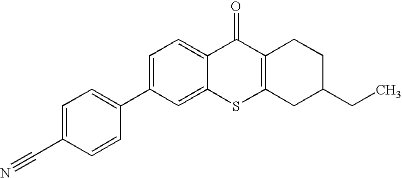

MS (EI+): 345 (M);

¹H-NMR (200 MHz, CDCl₃): δ=1.00 (t, J=7.5 Hz; 3H); 1.36-1.52 (m, 3H); 1.63-1.82 (m, 1H); 1.99-2.13 (m, 1H); 2.36-2.64 (m, 2H); 2.68-2.81 (m, 1H); 2.87-3.02 (m, 1H); 7.65-7.82 (m, 6H); 8.57-8.61 (m, 1H).

### EXAMPLE 1-27

### 3-(3,3-Dimethyl-9-oxo-2,3,4,9-tetrahydro-1H-thioxanthen-6-yl)benzonitrile

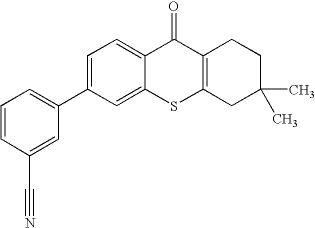

MS (EI+): 345 (M);

¹H-NMR (200 MHz, CDCl₃): δ=1.06 (s, 6H); 1.65 (t, J=6.5 Hz; 2H); 2.51 (s, 2H); 2.75 (t, J=6.5 Hz; 2H); 7.57-7.74 (m, 4H); 7.85-7.93 (m, 2H); 8.58-8.62 (m, 1H).

### EXAMPLE 1-28

### 3-(9-Oxo-3-spirobutyl-2,3,4,9-tetrahydro-1H-thioxanthen-6-yl)benzomitrile

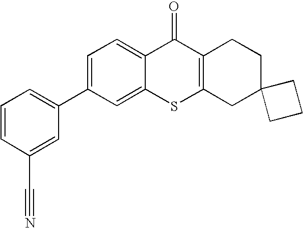

MS (CI+): 358 (M+H);

¹H-NMR (300 MHz, CDCl₃): δ=1.81-2.01 (m, 8H); 2.72-2.78 (m, 4H); 7.58-7.73 (m, 4H); 7.86-7.90 (m, 1H); 7.93 (s, 1H); 8.59 (d, J=8.5 Hz; 1H).

### EXAMPLE 1-29

### 3-(3-Ethyl-9-oxo-2,3,4,9-tetrahydro-1H-thioxanthen-6-yl)benzonitrile

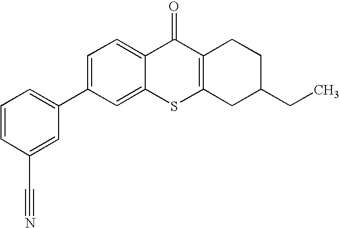

MS (CI+): 346 (M+H);

¹H-NMR (200 MHz, CDCl₃): δ=1.01 (t, J=7.5 Hz; 3H); 1.34-1.52 (m, 3H); 1.64-1.82 (m, 1H); 2.01-2.13 (m, 1H); 2.36-2.63 (m, 2H); 2.68-2.81 (m, 1H); 2.87-3.02 (m, 1H); 7.57-7.73 (m, 4H); 7.86-7.93 (m, 2H); 8.57-8.62 (m, 1H).

### EXAMPLE 1-30

### 3-Ethyl-6-(3-pyridinyl)-1,2,3,4-tetrahydro-9H-thioxanthen-9-one

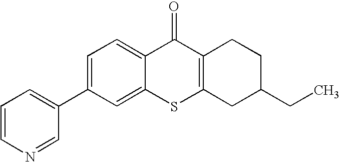

MS (DCI+): 322 (M+H);

¹H-NMR (200 MHz, CDCl₃): δ=1.01 (t, J=7.5 Hz; 3H); 1.36-1.52 (m, 3H); 1.65-1.81 (m, 1H); 1.99-2.13 (m, 1H); 2.36-2.64 (m, 2H); 2.68-2.82 (m, 1H); 2.87-3.02 (m, 1H); 7.39-7.45 (m, 1H); 7.66-7.70 (m, 2H); 7.92-7.98 (m, 1H); 8.58-8.6 (m, 2H); 8.92 (m, 1H).

### EXAMPLE 1-31

### 3,3-Dimnethyl-6-(3-pyridinyl)-1,2,3,4-tetrahydro-9H-thioxanthen-9-one

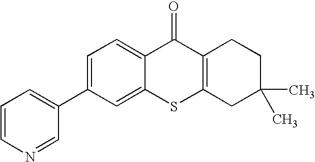

MS (CI+): 322 (M+H);

¹H-NMR (300 MHz, CDCl₃): δ=1.06 (s, 6H); 1.65 (t, J=6.5 Hz; 2H); 2.50 (s, 2H); 2.75 (t, J=6.5 Hz; 2H); 7.40-7.45 (m, 1H); 7.67-7.69 (m, 2H); 7.93-7.97 (m, 1H); 8.61 (dd, J=8 Hz, 1 Hz; 1H); 8.67 (dd, J=4.5 Hz, 1.5 Hz; 1H); 8.90 (m, 1H).

### EXAMPLE 1-32

### 6-(3-Pyridinyl)-3-spirobutyl-1,2,3,4-tetrahydro-9H-thioxanthen-9-one

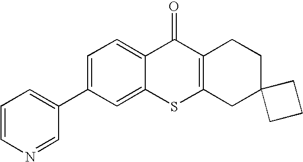

MS (CI+): 334 (M+H);

¹H-NMR (300 MHz, CDCl₃): δ=1.81-2.02 (m, 8H); 2.70-2.77 (m, 4H); 7.40-7.45 (m, 1H); 7.66-7.70 (m, 2H); 7.92-7.96 (m, 1H); 8.59 (d, J=8.5 Hz; 1H); 8.66-8.68 (dd, J=4.5 Hz, 1.5 Hz; 1H); 8.91 (d, J=2 Hz; 1H).

### EXAMPLE 1-33

### 3-Ethyl-6-(4-pyridinyl)-1,2,3,4-tetrahydro-9H-thioxanthen-9-one

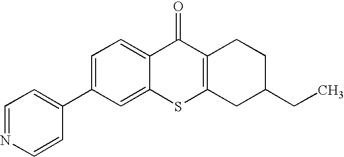

MS (CI+): 322 (M+H);

¹H-NMR (300 MHz, CDCl₃): δ=1.01 (t, J=7.5 Hz; 3H); 1.38-1.49 (m, 3H); 1.68-1.79 (m, 1H); 2.02-2.11 (m, 1H); 2.39-2.59 (m, 2H); 2.71-2.78 (m, 1H); 2.89-2.98 (m, 1H); 7.55-7.58 (m, 2H); 7.70-7.75 (m, 2H); 8.59-8.16 (d, J=8.5 Hz; 1H); 8.72 (m, 2H).

### EXAMPLE 1-34

### 3-(3-Ethyl-9-oxo-2,3,4,9-tetrahydro-1H-thioxanthen-6-yl)-1,2,4-oxadiazol-5(4H)-one

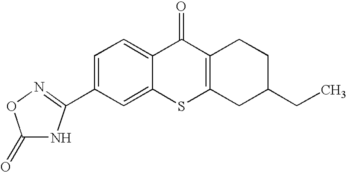

100 mg (0.33 mmol) of 3-ethyl-N′-hydroxy-9-oxo-2,3,4,9-tetrahydro-1H-thioxanthene-6-carboximidamide (Example I-7) and 0.03 ml (0.36 mmol) of pyridine are dissolved in 2 ml of DMF under argon. The solution is cooled to about 0° C., 0.06 ml (0.33 mmol) of 2-ethylhexyl chlorofonnate is added dropwise, and the mixture is stirred at about 0° C. for 30 min. The mixture is put then put into about 50 ml of water, extracted three times with 50 ml of ethyl acetate each time, and the organic phases are combined and dried and concentrated in a rotary evaporator.

The remaining residue is taken up in xylene and heated to boiling for 8 h.

The suspension formed after standing at normal temperature overnight is mixed with 20 ml of diethyl ether, the mixture is stirred for 5 min, and then the solid is filtered off, thoroughly washed with about 30 ml of diethyl ether and dried. 68.2 mg (63%) of the target compound are obtained in this way.

MS (ESI): 329 (M+H);

¹H-NMR (300 MHz, DMSO): δ=0.95 (t, J=7.5 Hz; 3H); 1.34-1.44 (m, 3H); 1.61-1.73 (m, 1H); 1.92-2.01 (m, 1H); 2.33-2.46 (m, 2H); 2.70-2.82 (m, 2H); 7.93 (m, 1H); 8.17 (m, 1H); 8.44 (m, 1H); 13.15 (s, b, 1H).

The following were prepared in analogy to the method of Example 1-34:

### EXAMPLE 1-35

### 3-(12-Oxo-6,8,9,10,11,12-hexahydro-7H-cycloocta[b]thiochromen-3-yl)-1,2,4-oxadiazol-5(4H)-one

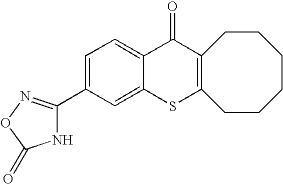

MS (ESI): 329 (M+H);

¹H-NMR (300 MHz, DMSO): δ=1.34-1.49 (m, 4H); 1.54-1.64 (m, 2H); 1.74-1.82 (m, 2H); 2.86-2.93 (m, 4H); 3.25-3.36 (s, b, 1H); 7.93-7.97 (dd, J=8.5 Hz, 1.5 Hz, 1H); 8.19 (d, J=1.5 Hz; 1H); 8.46 (d, J=8.5 Hz; 1H).

### EXAMPLE 1-36

### 3-(3,3-Dimnethyl-9-oxo-2,3,4,9-tetrahydro-1H-thioxanthen-6-yl)-1,2,4-oxadiazol-5(4H)-one

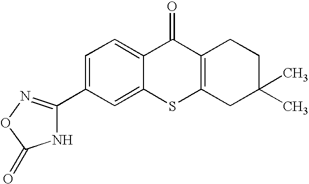

MS (ESI): 329 (M+H);

¹H-NMR (200 MHz, DMSO): δ=1.00 (s, 6H); 1.59 (t, J=6.5 Hz; 2H); 2.49-2.62 (m, 3H); 7.92-7.97 (dd, J=8.5 Hz, 1.5 Hz, 1H); 8.17 (s, 1H); 8.45 (d, J=8.5 Hz; 1H), 13.15 (s, b, 1H).

### EXAMPLE 1-37

### 2-[(3-Ethyl-9-oxo-2,3,4,9-tetrahydro-1-thioxanthen-6-yl)oxy]acetamide

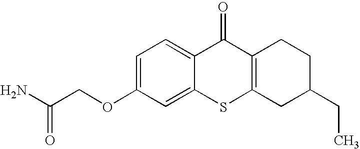

Under argon, 50 mg (0.19 mmol) of 3-ethyl-6-hydroxy-1,2,3,4-tetrahydro-9H-thioxanthen-9-one (Example I-6), 53 mg (0.38 mmol) of bromoacetamide, 188 mg (0.58 mmol) of cesium carbonate and about 3 mg of potassium iodide are mixed in 3 ml of 2-butanone, and the resulting reaction mixture is heated to reflux overnight.

For working up, 30 ml of ethyl acetate are added to the mixture, the insoluble solid is removed, and the filtrate is concentrated in a rotary evaporator and purified by preparative HPLC.

37.7 mg (62%) of the target compound are obtained in this way.

MS (ESI): 318 (M+H);

¹H-NMR (300 MHz, CDCl₃): δ=0.99 (m, 3H); 1.34-1.46 (m, 3H); 1.72-1.78 (m, 1H); 1.97-2.07 (m, 1H); 2.33-2.56 (m, 2H); 2.64-2.73 (m, 1H); 2.84-2.94 (m, 1H); 4.58 (d, 2H); 5.70 (s, 1H); 6.50 (s, 1H); 6.93 (m, 1H); 7.05 (m, 1H); 8.46 (m, 1H).

The following were prepared in analogy to the method of Example 1-37.

### EXAMPLE 1-38

### [(3-Ethyl-9-oxo-2,3,4,9-tetrahydro-1-thioxanthen-6-yl)-oxy]-acetonitrile

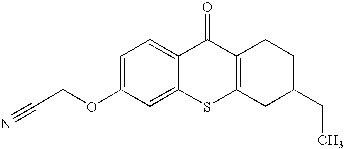

MS (CI+): 300 (M+H);

¹H-NMR (200 MHz, CDCl₃): δ=0.99 (t, J=7.5 Hz; 3H); 1.34-1.50 (m, 3H); 1.62-1.76 (m, 1H); 1.96-2.09 (m, 1H); 2.30-2.58 (m, 2H); 2.63-2.74 (m, 1H); 2.83-2.97 (m, 1H); 4.86 (s, 2H); 7.00 (d, J=2.5 Hz; 1H); 7.06-7.12 (dd, J=9 Hz, 2.5 Hz; 1H); 8.49 (d, J=9 Hz; 1H).

### EXAMPLE 1-39

### 6-Ethoxy-3-ethyl-1,2,3,4-tetrahydro-9H-thioxanthen-9-one

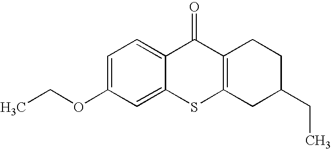

MS (CI+): 289 (M+H);

¹H-NMR (300 MHz, CDCl₃): δ=0.99 (t, J=7.5 Hz; 3H); 1.34-1.48 (m, 6H); 1.66-1.74 (m, 1H); 1.98-2.07 (m, 1H); 2.32-2.56 (m, 2H); 2.63-2.71 (m, 1H); 2.86-2.96 (m, 1H); 4.08-4.13 (quart., J=7 Hz; 2H); 6.88 (d, J=2.5 Hz; 1H); 6.99-7.03 (dd, J=9 Hz, 2.5 Hz; 1H); 8.39-8.43 (d, J=9 Hz; 1H).

### EXAMPLE 1-40

### 3-Ethyl-9-oxo-2,3,4,9-tetrahydro-1H-thioxanthen-6-yl methanesulfonate

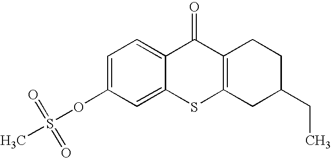

70 mg (0.27 mmol) of 3-ethyl-6-hydroxy-1,2,3,4-tetrahydro-9H-thioxanthen-9-one (Example I-6) are introduced under argon into dichloromethane, and initially 43 mg (0.13 mmol) of tetra-n-butylammonium bromide and 120 mg of 45 percent strength sodium hydroxide solution and, after 10 min 34 mg (0.30 mmol) of methanesulfonyl chloride are added. After stirring at normal temperature for 1.5 h, for working up 0.5 ml of buffer of pH 7 is added, the resulting mixture is sucked through a cartridge with 1 g of Extrelut/silica gel, the cartridge is washed with ethyl acetate, and the eluate is concentrated. Purification of the crude product by preparative HPLC affords 62.9 mg (69%) of the target compound.

MS (CI+): 339 (M+H);

¹H-NMR (300 MHz, CDCl₃): δ=0.99 (t, J=7.5 Hz; 3H); 1.36-1.48 (m, 3H); 1.64-1.77 (m, 1H); 2.00-2.09 (m, 1H); 2.36-2.57 (m, 2H); 2.67-2.76 (m, 1H); 2.86-2.95 (m, 1H); 3.22 (s, 3H); 7.32-7.36 (dd, J=9 Hz, 2.5 Hz; 1H); 7.48 (d, J=2.5 Hz; 1H); 8.54-8.57 (d, J=9 Hz; 1H).

The following were prepared in analogy to the method of Example 1-40:

### EXAMPLE 1-41

### 3-Ethyl-9-oxo-2,3,4,9-tetrahydro-1-thioxanthen-6-yl 4,4,4-trifluoro-1-butane-sulfonate

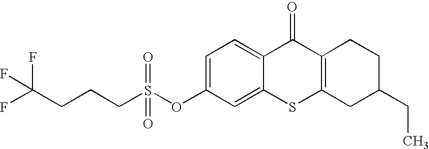

MS (CI+): 435 (M+H);

¹H-NMR (300 MHz, CDCl₃): δ=0.99 (t, J=7.5 Hz; 3H); 1.36-1.48 (m, 3H); 1.64-1.77 (m, 1H); 2.00-2.09 (m, 1H); 2.26-2.58 (m, 6H); 2.68-2.76 (m, 1H); 2.86-2.95 (m, 1H); 3.41 (t, J=7 Hz; 2H); 7.30-7.33 (dd, J=9 Hz, 2.5 Hz; 1H); 7.45 (d, J=2.5 Hz; 1H); 8.53-8.56 (d, J=9 Hz; 1H).

### EXAMPLE 1-42

### 3-[(3-Ethyl-9-oxo-2,3,4,9-tetrahydro-1-thioxanthen-6-yl)oxy]benzonitrile

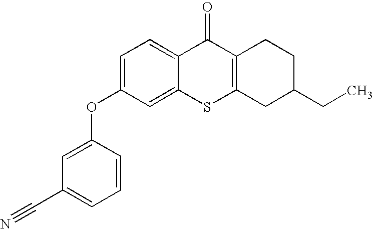

Under argon, 250 mg (0.77 mmol) of 6-bromo-3-ethyl-1,2,3,4-tetrahydro-9H-thioxanthen-9-one (Example I-1), 276 mg (2.32 mmol) of 3-cyanophenol and 214 mg (1.55 mmol) of potassium carbonate are introduced into pyridine and briefly heated to 140° C. The mixture is allowed to cool slightly again, and 147 mg (0.77 mmol) of copper(I) iodide are added. The mixture is then stirred at about 140° C. for 48 h.

For working up, the pyridine is removed in a rotary evaporator by twice taking up the initially remaining residue in toluene and again evaporating. The residue is taken up in ethyl acetate, and the mixture is extracted with 5N hydrochloric acid and washed with sodium bicarbonate solution and water. After drying over magnesium sulfate and concentrating, the oily crude product is absorbed onto 0.5 g of silica gel and purified by column chromatography on about 40 g of silica gel with a cyclohexane/ethyl acetate mobile phase gradient from 20:1 to 2:1. 95.3 mg (34.1%) of the target compound are obtained in this way.

MS (CI+): 362 (M+H);

¹H-NMR (300 MHz, CDCl₃): δ=0.99 (t, J=7.5 Hz; 3H); 1.34-1.47 (m, 3H); 1.64-1.76 (m, 1H); 1.98-2.07 (m, 1H); 2.34-2.57 (m, 2H); 2.64-2.73 (m, 1H); 2.86-2.96 (m, 1H); 7.03 (d, J=2 Hz; 2H); 7.08-7.11 (m, 1H); 7.28-7.37 (m, 1H); 7.47-7.54 (m, 2H); 8.50-8.53 (m, 1H).

The following were prepared in analogy to the method of Example 1-42:

### EXAMPLE 1-43

### 3-[(3,3-Dimethyl-9-oxo-2,3,4,9-tetrahydro-1H-thioxanthen-6-yl)oxy]benzonitrile

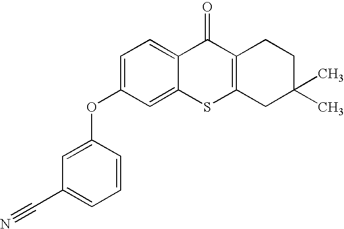

MS (EI+): 361 (M);

¹H-NMR (300 MHz, CDCl₃): δ=1.04 (s, 6H); 1.63 (t, J=6.5 Hz; 2H); 2.45 (s, 2H); 2.71 (t, J=6.5 Hz; 2H); 7.03 (d, J=2.5 Hz; 1H); 7.08-7.12 (dd, J=9 Hz, 2.5 Hz; 1H); 7.29-7.35 (m, 2H); 7.47-7.52 (m, 2H); 8.52 (d, J=9 Hz; 1H).

The following are prepared in analogy to the method of Example 1-3:

### EXAMPLE 1-44

### 3,3-Diethyl-6-(1H-tetrazol-5-yl)-1,2,3,4-tetrahydro-9H-thioxanthen-9-one

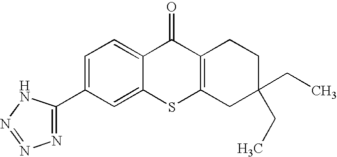

HPLC: Rₜ=4.95 min;

¹H-NMR (400 MHz, DMSO): δ=0.82 (t, 6H); 1.33 (mc, 4H); 1.60 (t, 2H); 2.45-2.60 (m, 4H); 3.1-3.6 (s, b, 1H); 8.17 (dd, 1H); 8.40 (s, 1H); 8.49 (d, 1H).

The following are prepared in analogy to the method of Example 1-10:

### EXAMPLE 1-45

### 3,3-Diethyl-6-(2-thienyl)-1,2,3,4-tetrahydro-9H-thioxanthen-9-one

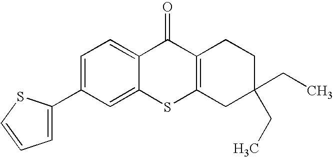

HPLC: Rₜ=7.05 min;

MS (ESI+): 355 (M+H);

¹H-NMR (300 MHz, CDCl₃): δ=0.83 (t, 6H); 1.2-1.5 (m, 4H); 1.65 (t, 2H); (s, br, 2H); 2.67 (t, 2H); 7.14 (dd, 1H); 7.40 (dd, 1H); 7.46 (dd, 1H); 7.69 (s, 1H); 7.71 (dd, 1H); 8.49 (d, 1H).

### EXAMPLE 1-46

### 6-(2-Thienyl)-3-spirohexyl-1,2,3,4-tetrahydro-9H-thioxanthen-9-one

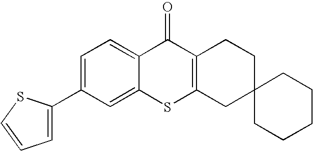

MS (EI+): 366 (M);

HPLC: Rₜ=6.55 min;

¹H-NMR (200 MHz, CDCl₃): δ=1.3-1.6 (m, 10H); 1.71 (t, 2H); 2.53 (s, br, 2H); 2.69 (t, br, 2H); 7.14 (dd, 1H); 7.40 (dd, 1H); 7.46 (dd, 1H); 7.65-7.75 (m, 2H); 8.49 (d, 1H).

### EXAMPLE 1-47

### 6-(3-Cyanophenyl)-3,3-diethyl-1,2,3,4-tetrahydro-9H-thioxanthen-9-one

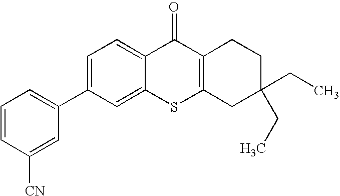

MS (ESI+): 374 (M+H);

HPLC: Rₜ=5.59 min;

¹H-NMR (300 MHz, CDCl₃): δ=0.87 (t, 6H); 1.23-1.52 (m, 4H); 1.67 (t, 2H); 2.50 (s, br, 1H); 2.69 (t, 2H); 7.55-7.75 (m, 4H); 7.88 (dt, 1H); 7.90-7.95 (m, 1H); 8.60 (d, 1H).

### EXAMPLE 1-48

### 6-(3-Cyanophenyl)-3-spirohexyl-1,2,3,4-tetrahydro-9H-thioxanthen-9-one

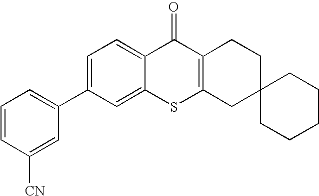

MS (EI+): 385 (M);

HPLC: Rₜ=6.11 min;

¹H-NMR (200 MHz, CDCl₃): δ=1.35-1.6 (m, 10H); 1.73 (t, 2H); 2.56 (s, br, 2H); 2.71 (t, br, 2H); 7.55-7.76 (m, 4H); 7.83-7.96 (dd, 2H); 8.59 (dd, 1H).

### EXAMPLE 1-49

### 6-(Hydroxymethyl)-3,3-dimethyl-1,2,3,4-tetrahydro-9H-thioxanthen-9-one

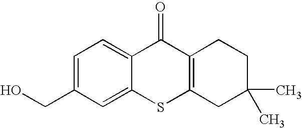

269 mg (1.19 mmol) of diisopropylethylamine and, after stirring at normal temperature for 5 min, sodium borohydride are added under argon to a solution of 500 mg (1.73 mmol) of 3,3-dimethyl-9-oxo-2,3,4,9-tetrahydro-1H-thioxanthene-6-carboxylic acid and 844 mg (1.91 mmol) of benzotriazolyloxytris(dimethylamino)phosphonium hexafluorophosphate in 8.5 ml of tetrahydrofuran. After 80 min, the solvent is removed in vacuo, the residue is taken up in diethyl ether, and the organic phase is washed successively with 1N hydrochloric acid, saturated sodium bicarbonate solution and saturated brine, dried over magnesium sulfate and concentrated in vacuo. Chromatography on silica gel (cyclohexane/ethyl acetate mobile phase gradient 5:1-2:1) results in 302 mg (63%) of the target compound.

MS (ESI+): 275 (M+H);

HPLC: Rₜ=4.47 min;

¹H-NMR (300 MHz, CDCl₃): δ=1.04 (s, 6H); 1.63 (t, 2H); 1.94 (s, br, 1H); 2.47 (s, br, 2H); 2.72 (t, 2H); 7.41 (d, br, 1H); 7.53 (s, br, 1H); 8.46 (d, 1H).

### EXAMPLE 1-50

### 6-(Chloromethyl)-3,3-dimethyl-1,2,3,4tetrahydro-9H-thioxanthen-9-one

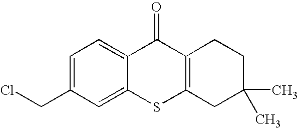

1.00 g (3.64 mmol) of 6-(hydroxymethyl)-3,3-dimethyl-1,2,3,4-tetrahydro-9H-thioxanthen-9-one and 1.91 g (7.29 mmol) of triphenylphosphine are introduced into 7.2 ml of dichloromethane, and the mixture is cooled in a dry ice/acetone bath to minus 10° C. Then 1.12 g (7.29 mmol) of tetrachloromethane are added to the colorless suspension which forms, and the reaction is left to stir overnight while thawing. It is diluted with dichloromethane, washed with saturated sodium bicarbonate solution, water and with saturated brine and dried over magnesium sulfate. The solvent is removed in vacuo, and the residue is adsorbed onto silica gel and filtered through silica gel (mobile phase:cyclohexane/ethyl acetate 5:1). 1.02 g (95%) of the target compound are obtained in this way.

MS (ESI+): 293 (M+H);

HPLC: Rₜ=5.35 min;

¹H-NMR (300 MHz, CDCl₃): δ=0.99 (s, 6H); 1.63 (t, 2H); 2.48 (s, 2H); 2.72 (t, 2H); 4.64 (s, 2H); 7.47 (dd, 1H); 7.53 (s, br, 1H); 8.49 (d, 1H).

### EXAMPLE 1-51

### 2-(3,3-Dimethyl-9-oxo-2,3,4,9-tetrahydro-1H-thioxanthen-6-yl)-N-isobutyl-N-methylacetamide

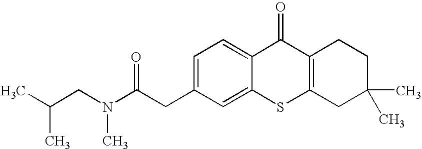

26 mg (0.18 mmol) of 1-hydroxy-1H-benzotriazole hydrate and then 36 mg (0.18 mmol) of N-(3-dimethylaminopropyl)-N′-ethylcarbodiimide hydrochloride are added to a solution of 50 mg (0.17 mmol) of (3,3-dimethyl-9-oxo-2,3,4,9-tetrahydro-1H-thioxanthen-6-yl)acetic acid and 43 mg (0.33 mmol) of dilsopropylethylamine while cooling in ice. The reaction is left to stir for 15 min and then 16 mg (0.18 mmol) of N-isobutyl-N-methylamine are added. The mixture is stirred for 19 h during which the mixture is allowed to reach normal temperature. For working up, the mixture is diluted with dichloromethane, washed with water and saturated sodium chloride solution and dried over magnesium sulfate. Removal of the solvent in vacuo and chromatography (preparative HPLC, acetonitrile/water 30:70-95:5) affords 54 mg (88%) of the target compound.

MS (ESI+): 372 (M+H);

HPLC: Rₜ=4.95 min;

¹H-NMR (400 MHz, CDCl₃): δ=0.87, 0.90 (2 d, total 6H); 1.03 (s, 6H); 1.62 (t, 2H) 1.96 (mc, 1H), 2.46 (s, 2H), 2.71 (t, 2H); 2.96, 2.97 (2s, total 3H); 3.11, 3.23 (2d, total 2H), 3.79, 3.80 (2s, total 2H); 7.30-7.40 (2 dd, total 1H); 7.43, 7.44 (2s total 1H); 8.45, 8.45 (2d, total 1H).

### STARTING COMPOUNDS II

HPLC methods:
Method A: Eluent: A=0.5% HClO₄ in water, B=acetonitrile; gradient: 0.5 min 98% A, 2% B, 4.5 min 10% A, 90% B, 6.7 min 98% A, 2% B; flow rate: 0.75 ml/min; column temperature: 30° C.; UV detection: 210 nm; colum: Kromasil C18 (60*2 mm)
Method C: Eluent: A=0.1% formic acid in acetonitrile, B=0.1% formic acid in water; gradient: 0 min 10% A, 90% B, 4.0 min 90% A, 10% B, 6.1 min 10% A, 90% B, 7.50 min 10% A, 90% B; flow rate: 0.5 ml/min 0.00-6.10 min, 0 ml/min 6.10-7.5 min, column temperature: 40° C.; UV detection: 208-400 nm; column: Symmetry C18 (50*2.1 mm), 3.5 μm
Method D: Eluent: A=acetonitrile, B=0.3 g of 30% HCl in 11 of water; gradient: 3 min 90% B, 10% A, flow rate 0.9 ml/min, 6 min 90% A, 10% B, flow rate 1.2 ml/min, column temperature: 50° C.; column: Symmetry C18 (150*2.1 mm); for other parameters, see method A

### EXAMPLE 11-1

### 6-(Benzyloxy)-3-ethyl-1,2,3,4-tetrahydro-9H-thioxanthen-9-one

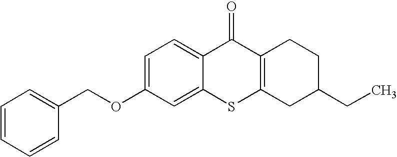

60 mg (0.23 mmol) of 3-ethyl-6-hydroxy-1,2,3,4-tetrahydro-9H-thioxanthen-9-one, 39.4 mg (0.23 mmol) of benzyl bromide and 35 mg (0.25 mmol) of powdered potassium carbonate in 2 ml of acetonitrile are heated to reflux under argon at RT overnight. Concentration is followed by to add dichloromethane, washing with dilute hydrochloric acid and dilute sodium hydroxide solution, and the organic phase is dried over magnesium sulfate. Concentration results in the crude product as an oil which is freed of solvent residues under high vacuum. Trituration with a little isopropanol results in a solid which is dried in air; yield 37.3 mg (46%), m.p. 94° C.

MS (ESI): 351 (M+H)⁺.

HPLC: 97.4%, retention time 6.06 min (Method A).

The following were prepared in analogy to the method of Example II-1:

### EXAMPLE II-2

### 3-Ethyl-6-(2-phenylethoxy)-1,2,3,4-tetrahydro-9H-thioxanthen-9-one

Yield: 42%, m.p. 105° C., HPLC: 100%, MS (ESI): 365 (M+H)⁺.

### EXAMPLE II-3

### 3-{[(3-Ethyl-9-oxo-2,3,4,9-tetrahydro-1H-thioxanthen-6-yl)oxy]methyl}benzonitrile

Yield: 72%, m.p. 158° C., HPLC: 100%, MS (ESI): 375 (M)⁺.

### Example II-4a and Example II-4b

### N-(3-Ethyl-9-oxo-2,3,4,9-tetrahydro-1-thioxanthen-6-yl)acetamide and 6-amino-3-ethyl-1,2,3,4-tetrahydro-9H-thioxanthen-9-one

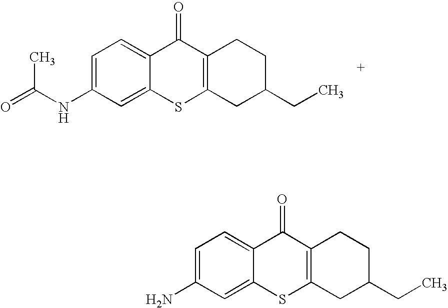

15 ml of polyphosphoric acid, 1.11 g (6.6 mmol) of N-(3-mercaptophenyl)acetamide and 1.45 g (7.3 mmol) of ethyl 4-ethyl-2-oxocyclohexanecarboxylate are heated at 90° C. under argon for 30 minutes. After cooling to room temperature, 100 ml of ice-water are added, and the mixture is stirred for 30 minutes. Extractive working up with ethyl acetate and washing of the organic phase with 1 N sodium hydroxide solution and saturated brine affords, after drying over sodium sulfate and concentration, a crude product which is purified by separation by column chromatography (silica gel, cyclohexane/ethyl acetate mixtures). 135 mg (7%) of N-(3-ethyl-9-oxo-2,3,4,9-tetrahydro-1H-thioxanthen-6-yl)acetamide are obtained as a solid, m.p. 243° C.

HPLC: 99%, retention time 4.61 min (Method A),

MS (ESI): 302 (M+H)⁺.

As a further fraction, 77 mg (4.5%) of 6-amino-3-ethyl-1,2,3,4-tetrahydro-9H-thioxanthen-9-one are obtained as a colorless solid, m.p. 187° C.

HPLC: 100%, retention time 4.51 min (Method A),

MS (ESI): 260 (M+H)⁺.

The following were prepared in analogy to the method of Examples II-4a and II-4b:

### EXAMPLE II-5

### 6-Amino-3,3-dimethyl-1,2,3,4-tetrahydro-9H-thioxanthen-9-one

The title compound is obtained from 3-aminothiophenol and ethyl 4,4-dimethyl-2-oxocyclohexane carboxylate in polyphosphoric acid (43%).

m.p. 210° C.

HPLC: 100%, retention time 4.44 min (Method A),

MS (EI pos): 259 (M)⁺.

### EXAMPLE II-6

### N-(3-Ethyl-9-oxo-2,3,4,9-tetrahydro-1H-thioxanthen-6-yl)benzenesulfonaimide

The title compound is obtained in analogy to Example 2-11 from benzenesulfonyl chloride and 6-amino-3-ethyl-1,2,3,4-tetrahydro-9H-thioxanthen-9-one.

Yield: 76% colorless crystals.

m.p. 264° C.

HPLC: 100%, retention time 5.20 min (Method A),

MS (ESI): 400 (M+H)⁺.

### EXEMPLARY EMBODIMENTS 2

### EXAMPLE 2-1

### 2-{4-[(3-Ethyl-9-oxo-2,3,4,9-tetrahydro-H-thioxanthen-6-yl)oxy]butyl}-1,2-benzisothiazol-3(2H)-one 1,1-dioxide

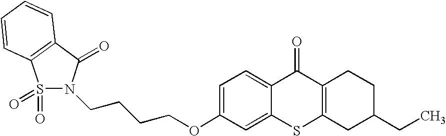

Under argon, 60 mg (0.23 mmol) of 3-ethyl-6-hydroxy-1,2,3,4-tetrahydro-9H-thioxanthen-9-one are dissolved in 1 ml of tetrahydrofuran and, after addition of 0.34 ml of a 1M solution of potassium tertiary butoxide in tetrahydrofuran, shaken at room temperature for 30 min. 110 mg (0.35 mmol) of 2-(4-bromobutyl)-1,2-benzisothiazol-3(2H)-one 1,1-dioxide are added; the solution is shaken at 65° C. overnight.

After cooling to room temperature, about 130 mg of PS-thiophenol (from Argonaut, 1.4 mmol/g) are added, and shaken at room temperature for 30 min. The solution is filtered, the solid is washed with 0.5 ml of dimethylformamide, and the filtrate is concentrated under high vacuum.

Purification takes place by preparative HPLC (reverse phase, acetonitrile/water mixtures).

Yield: 22.4 mg (20%).

MS (ESI+): 498 (M+H)⁺.

The compounds listed in the following table are obtained in the same way:

| | | | HPLC Method C, |
|---|---|---|---|
| | | Mass (ESI+) | retention time |
| Ex. No. | Structure | [M + H]⁺ | in min |
| 2-2 | 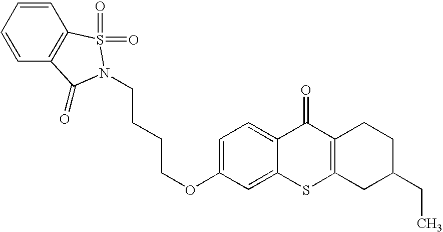 | 498 | 5.22 |
| 2-3 | 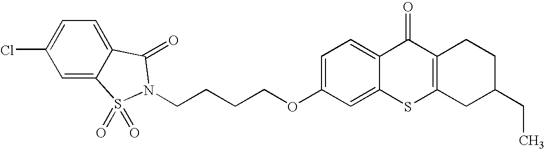 | 532 | 5.47 |
| 2-4 | 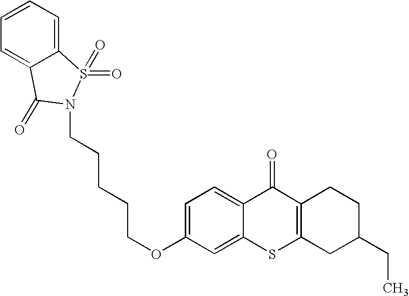 | 512 | 5.41 |
| 2-5 | 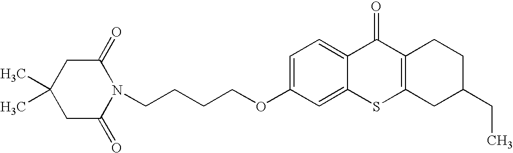 | 456 | 5.17 |
| 2-6 | 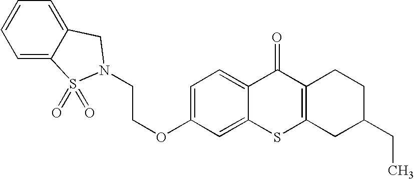 | 456 | 4.76 |
| 2-7 | 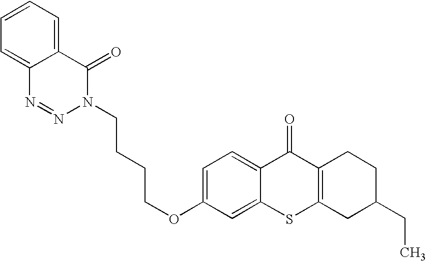 | 462 | 5.20 |
| 2-8 | 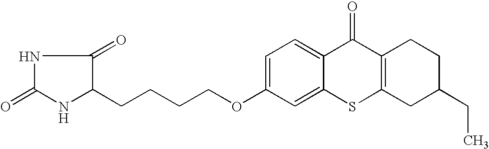 | 415 | 4.13 |

### EXAMPLE 2-9

### (3-Ethyl-9-oxo-2,3,4,5-tetrahydro-1H-thioxanthen-6-yl)2-(1-naphthyl)ethane-sulfonate

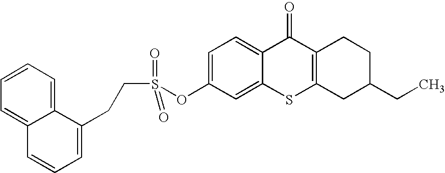

31 mg (0.1 mmol) of tetrabutylammmonium bromide and 86 mg of 45% aqueous sodium hydroxide solution are added to a solution of 50 mg (0.19 mmol). of 3-ethyl-6-hydroxy-1,2,3,4-tetrahydro-9H-thioxanthen-9-one in 1 ml of dichloromethane. After stirring at room temperature for 10 minutes, 59 mg (0.23 mmol) of 2-naphthylethanesulfonyl chloride are added. Stirring at room temperature for 1.5 hours is followed by dilution with dichloromethane, and the organic phase is washed with water. Filtration through silica gel, concentration and chromatography of the crude product (silica gel, toluene/ethyl acetate 10/1) affords the title compound (37%)

HPLC: 97%, retention time 5.92 min (Method A),

MS (ES): 479 (M+H)⁺.

The following are obtained in analogy to the method of

### EXAMPLE 2-9:

### EXAMPLE 2-10

### 3-Ethyl-9-oxo-2,3,4,9-tetrahydro-1H-thioxanthen-6-yl dimethylsulfamate

From dimethylamidosulfonyl chloride; yield: 57%

HPLC: 100%, retention time 5.23 min (Method A),

MS (ESI): 368 (M+H)⁺.

### EXAMPLE 2-11

### N-(3,3-Dimethyl-9-oxo-2,3,4,9-tetrahydro-1H-thioxanthen-6-yl)methanesulfonamide

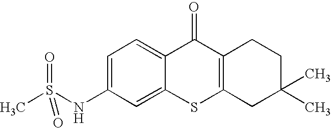

0.12 ml of pyridine and 0.03 ml (0.35 mmol) of methanesulfonyl chloride are added to a solution of 75 mg (0.3 mmol) of 6-amino-3,3-dimethyl-1,2,3,4-tetrahydro-9H-thioxanthen-9-one and 4 mg of 4-dimethylaminopyridine in 1.5 ml of dichloromethane at 0° C. Stirring at room temperature for 3 h is followed by dilution with dichloromethane and aqueous working up (1N hydrochloric acid, water, saturated brine). The organic phase is dried over sodium sulfate and then concentrated. The remaining solid is recrystallized from ethyl acetate. The crystals are washed with a little pentane and dried in vacuo.

Yield: 28 mg (29%) of colorless crystals.

m.p. 272° C.

HPLC: 100%, retention time 4.61 min (Method A),

MS (ESI): 338 (M+H)⁺.

The following are obtained in analogy to the method of Example 2-11:

### EXAMPLE 2-12

### N-(3,3-Dimethyl-9-oxo-2,3,4,9-tetrahydro-1H-thioxanthen-6-yl)benzenesulfonamide

From benzene sulfonyl chloride; yield: 73% colorless crystals.

m.p. 231° C.

HPLC: 100%, retention time 5.01 min (Method A),

MS (ESD: 400 (M+H)⁺.

### EXAMPLE 2-13

### N-(3,3-Dimethyl-9-oxo-2,3,4,9-tetrahydro-1H-thioxanthen-6-yl)-4-methylbenzenesulfonamide

From 4-methylbenzenesulfonyl chloride; yield: 60% colorless crystals.

m.p. 279° C.

HPLC: 100%, retention time 5.18 min (Method A),

MS (ESD: 414 (M+H)⁺.

### EXAMPLE 2-14

### 3,3-Dimethyl-9-oxo-2,3,4,9-tetrahydro-1H-thioxanthen-6-yl [2-(trifluoromethoxy)-phenyl]methanesulfonate

From 2-(trifluoromethoxy)phenyl]methanesulfonyl chloride; yield: 55% colorless crystals.

m.p. 249° C.

HPLC: 100%, retention time 5.32 min (Method A),

MS (ESI): 498 (M+H)⁺.

### EXAMPLE 2-15

### N-(3,3-Dimethyl-9-oxo-2,3,4,9-tetrahydro-1H-thioxanthen-6-yl)isopropylsulfonamide

From isopropylsulfonyl chloride (triple amount); yield: 14% colorless crystals.

m.p. 263° C.

HPLC: 100%, retention time 4.85 min (Method A),

MS (ESI): 366 (M+H)⁺.

### EXAMPLE 2-16

### N-(3,3-Dimethyl-9-oxo-2,3,4,9-tetrahydro-1-thioxanthen-6-yl)-N-methylbenzenesulfonamide

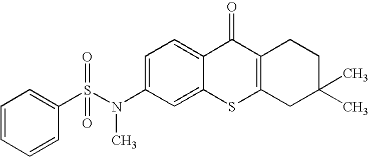

7.4 ml of 40% sodium hydride in paraffin oil are added to a solution of 41 mg (0.1 mmol) of N-(3,3-dimethyl-9-oxo-2,3,4,9-tetrahydro-1H-thioxanthen-6-yl)benzenesulfonamide in 1 ml of tetrahydrofuran, and the mixture is stirred under argon at room temperature for 1 hour. 16 mg (0.11 mmol) of methyl iodide are added. The mixture is stirred at 60° C. overnight. The same amount of methyl iodide is then added once again. A further 5 hours at 60° C. are followed by aqueous working up (ethyl acetate, water, sat. brine, drying over sodium sulfate). The crude product is purified by chromatography (silica gel, toluene/ethyl acetate 15:1).

Yield: 24 mg (53%).

HPLC: 92%, retention time 5.28 min (Method A),

MS (ESI): 414 (M+H)⁺.

### EXAMPLE 2-17

### N-(3-Ethyl-9-oxo-2,3,4,9-tetrahydro-1H-thioxanthen-6-yl)-N-methylbenzenesulfonamide

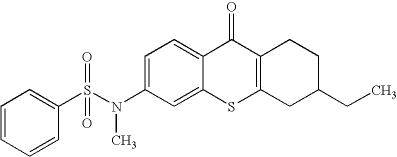

A solution of 41 mg (0.1 mmol) of N-(3-ethyl-9-oxo-2,3,4,9-tetrahydro-1H-thioxanthen-6-yl)benzenesulfonamide in 2 ml of dimethylformamide is mixed with 22 mg of methyl iodide, 3 mg of tetrabutylammmonium bisulfate and 1 g of potassium carbonate and stirred at room temperature for 3 days. Aqueous working up (ethyl acetate, water, sat. brine, drying over sodium sulfate) affords a crude product which is purified by chromatography (silica gel, toluene/ethyl acetate 10:1). Yield: 27 mg (64%).

HPLC: 98%, retention time 5.44 min (Method A),

MS (DCI NH3): 414 (M+H)⁺.

### EXAMPLE 2-18

### N-(Cyclopropylmethyl)-N-(3-ethyl-9-oxo-2,3,4,9-tetrahydro-1H-thioxanthen-6-yl)-benzenesulfonamide

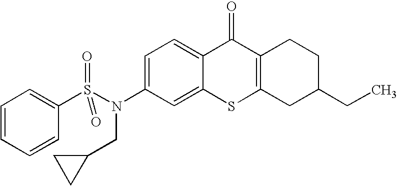

126 mg of 60% sodium hydride are freed of mineral oil with pentane under argon. The washed sodium hydride is mixed with 7 ml of dimethylformamide and then 1.05 g N-(3-ethyl-9-oxo-2,3,4,9-tetrahydro-1H-thioxanthen-6-yl)benzenesulfonamide are added. The mixture is stirred at room temperature overnight. The solution is made up to a total volume of 10.5 ml with dimethylformamide.

0.5 ml of the above solution (=0.125 mmol of sodium salt of the sulfonamide) is mixed with a solution of 25 mg (0.19 mmol) of cyclopropylmethyl bromide in 0.5 ml of dimethylformamide and shaken at 80° C. overnight. Cooling is followed by addition of 130 mg of PS-thiophenol (from Argonaut, 1.41 mmol/g) and shaking at room temperature for 1 h. Washing with dimethylformamide and concentration of the filtrate under high vacuum affords a crude product which is purified by HPLC (reverse phase, acetonitrile/water).

Yield: 7 mg (13%).

HPLC: 98%, retention time 3.78 min (Method D),

MS (ESI): 454 (M+H)⁺.

The following are obtained in analogy to the method of Example 2-18:

### EXAMPLE 2-19

### N-(3-Ethyl-9-oxo-2,3,4,9-tetrahydro-1H-thioxanthen-6-yl)-N-(2-methoxyethyl)benzenesulfonamide

From 2-methoxyethyl bromide; yield: 41%.

HPLC: 94%, retention time 3.54 min (Method D),

MS (ESI): 458 (M+H)⁺.

### EXAMPLE 2-20

### N-(3-Ethyl-9-oxo-2,3,4,9-tetrahydro-1H-thioxanthen-6-yl)-N-(phenylsulfonyl)glycine methyl ester

From methyl bromoacetate; yield: 54%

HPLC: 100%, retention time 3.43 min (Method D),

MS (ESI): 472 (M+H)⁺.

### EXAMPLE 2-21

### N-(3-Ethyl-9-oxo-2,3,4,9-tetrahydro-1H-thioxanthen-6-yl)-N-(2-phenylethyl)benzenesulfonamide

From 2-phenylethyl bromide; yield: 20%

HPLC: 97%, retention time 3.94 min (Method D),

MS (ESI): 504 (M+H)⁺.

### EXAMPLE 2-22

### N-(3-Ethyl-9-oxo-2,3,4,9-tetrahydro-1H-thioxanthen-6-yl)-N-propylbenzenesulfonamide

From 1-propyl bromide; yield: 22%

HPLC: 99%, retention time 3.81 min (Method D),

MS (ESI): 442 (M+H)⁺.

### EXAMPLE 2-23

### N-(3-Ethyl-9-oxo-2,3,4,9-tetrahydro-1H-thioxanthen-6-yl)-N-ethylbenzenesulfonamide

From ethyl bromide; yield: 14%

HPLC: 96%, retention time 3.67 min (Method D),

MS (ESI): 428 (M+H)⁺.

### EXAMPLE 2-24

### 3,3-Dimethyl-6-(1H-pyrrol-1-yl)-1,2,3,4-tetrahydro-9H-thioxanthen-9-one

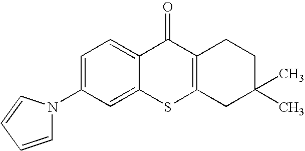

A solution of 136 mg (0.53 mmol) of 6-amino-3,3-dimethyl-1,2,3,4-tetrahydro-9H-thioxanthen-9-one in 1.5 ml of glacial acetic acid is mixed with 83 mg (0.63 mmol) of 2,5-dimethoxytetrahydrofuran and heated at 120° C. Thin-layer chromatography shows no precursor detectable after a short time. The reaction mixture is cooled to room temperature, diluted with ethyl acetate and subjected to aqueous working up (water, sodium bicarbonate, water, saturated brine). The crude product obtained after drying and concentration is purified by column chromatography (silica gel, cyclohexane/ethyl acetate mixtures). The resulting solid is digested with cyclohexane. Yield: 69 mg (42%) colorless crystals.

m.p. 169° C.

HPLC: 100%, retention time 5.34 min (Method A),

MS (DCI, NH₃): 310 (M+H)⁺.

The following is obtained in analogy to the method of Example 2-24

### EXAMPLE 2-25

### 3-Ethyl-6-(1H-pyrrol-1-yl)-1,2,3,4-tetrahydro-9H-thioxanthen-9-one

From 6-amino-3-ethyl-1,2,3,4-tetrahydro-9H-thioxanthen-9-one; yield: 28% colorless crystals.

m.p. 145° C.

HPLC: 99%, retention time 5.49 min (Method A),

MS (DCI, NH₃): 310 (M+H)⁺.

### EXEMPLARY EMBODIMENTS 3

The retention time of the prepared examples was determined by HPLC under the following conditions.

Column: Chromasil C18 60*2; volume injected 1.00 μl; flow rate: 0.75 ml/min; eluent: A=5 ml HClO₄/l H₂O, B=CH₃CN; gradient [t(min): A/B]: 0.5: 98/2; 4.5: 10/90; 6.5: 10/90; 6.7: 98/2; 7.5: 98:2.

LC-MS Method 1:

| Method: | | | MHZ 2Q | | | |
|---|---|---|---|---|---|---|
| Version No.: | | | 3 | | | |
| MS apparatus type: | | | Micromass Quattro LCZ | | | |
| | | | Ionization: ESI positive/negative | | | |
| HPLC apparatus type: | | | HP 1100 | | | |
| | | | UV detector DAD: 208-400 nm | | | |
| | | | Oven temp.: 40° C. | | | |
| Column: | | | Symmetry C 18 | | | |
| | | | 50 mm × 2.1 mm 3.5 μm | | | |
| Supplied by: | | | Waters | | | |
| Gradient: | Time | A: % | B: % | C: % | D: % | Flow |
| | 0.00 | 10.0 | 90.0 | — | — | 0.50 |
| | 4.00 | 90.0 | 10.0 | — | — | 0.50 |
| | 6.00 | 90.0 | 10.0 | — | — | 0.50 |
| | 6.10 | 10.0 | 90.0 | — | — | 1.00 |
| | 7.50 | 10.0 | 90.0 | — | — | 0.50 |

A: CH3CN + 0.1% formic acid
B: H2O + 0.1% formic acid
C: —
D: —

LC-MS Method 2:

| Method: | | | MHZ 2P | | | |
|---|---|---|---|---|---|---|
| Version No.: | | | 3 | | | |
| MS apparatus type: | | | Micromass Platform LCZ | | | |
| | | | Ionization: ESI positive/negative | | | |
| HPLC apparatus type: | | | HP 1100 | | | |
| | | | UV detector DAD: 208-400 nm | | | |
| | | | Oven temp.: 40° C. | | | |
| Column: | | | Symmetry C 18 | | | |
| | | | 50 mm × 2.1 mm 3.5 μm | | | |
| Supplied by: | | | Waters | | | |
| Gradient: | Time | A: % | B: % | C: % | D: % | Flow |
| | 0.00 | 10.0 | 90.0 | — | — | 0.50 |
| | 4.00 | 90.0 | 10.0 | — | — | 0.50 |
| | 6.00 | 90.0 | 10.0 | — | — | 0.50 |
| | 6.10 | 10.0 | 90.0 | — | — | 1.00 |
| | 7.50 | 10.0 | 90.0 | — | — | 0.50 |

A: CH3CN + 0.1% formic acid
B: H2O + 0.1% formic acid
C: —
D: —

LC-MS Method 3

Column: Symmetry C18 150*2.1; volume injected 2.00 μl; eluent: A=CH₃CN, B=0.3 g HCl (30%)/l H₂O; gradient [t(min): A/B]: 0: 10/90 flow rate: 0.9 m/min; 3.0: 90/10 flow rate: 1.2 ml/min; 6.0: 90/10; flow rate: 1.2 ml/min.

### EXAMPLE 3-1

### 3-Ethyl-N-(2-methylbutyl)-9-oxo-2,3,4,9-tetrahydro-1H-thioxanthene-6-carboxamide

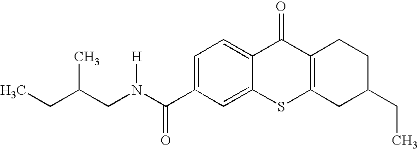

0.70 g (2.43 mmol) of 3-ethyl-9-oxo-2,3,4,9-tetrahydro-1H-thioxanthene-6-carboxylic acid and 3.22 g (7.28 mmol) of 1-benzotriazolyloxytris(dimethylamino)-phosphonium hexafluorophosphate in 44 ml of THF are stirred at 25° C. under argon for 15 min. Firstly 0.85 ml (6.07 mmol) of triethylamine and, after a firther 15 min, 1.73 ml (14.6 mmol) of 2-methylbutylamine are added dropwise to the solution, which is then stirred at 25° C. for 24 h. Dilution with dichloromethane is followed by addition of citric acid (5% strength in water). The phases are separated and the organic phase is washed with aqueous sodium bicarbonate solution. After the combined organic phases have been dried over sodium sulfate and filtered, the solvent is distilled out under reduced pressure. The residue is prepurified by chromatography on silica gel (0.04-0.063 mm) with dichloromethane/methanol 20:1 as mobile phase. Pure carboxamide is subsequently obtained by a second chromatography on silica gel (0.04-0.063 mm) with cyclohexane/ethyl acetate 2:1 as mobile phase.

Yield: 655 mg (75.5%).

R_{f} (CH₂Cl₂/MeOH 20/1)=0.52.

MS (DCI): 358 (M+H).

HPLC, retention time=5.23 min.

¹H-NMR (200 MHz, CDCl₃): δ=0.96 (t, 3H), 0.99 (d, 3H), 1.00 (t, 3H), 1.13-1.53 (m, 5H), 1.63-1.80 (m, 2H), 1.97-2.14 (m, 1H), 2.34-2.63 (m, 2H), 2.67-2.83 (m, 1H), 2.84-3.02 (m, 1H), 3.23-3.52 (m, 2H), 6.14-6.28 (m, 1H), 7.70 (dd, 1H), 8.00 (d, 1H), 8.54 (d, 1H).

The carboxamides in Table 1 were obtained by the process described for Example 3-1.

TABLE 1

| | | | Retention | Mass |
|---|---|---|---|---|
| Ex. No. | Structure | Yield | time (min) | (M + H)⁺ |
| 3-2 | 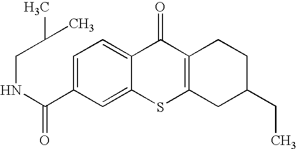 | 90.3% | 5.1 | 344 |
| 3-3 | 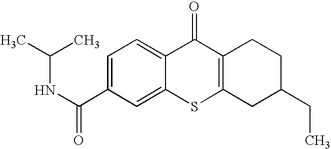 | 59.1% | 4.46 (LC-MS Method 2) | 330 |
| 3-4 | 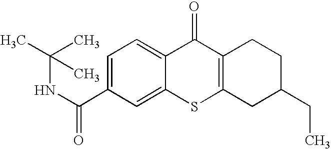 | 83.9% | 4.86 | 344 |
| 3-5 | 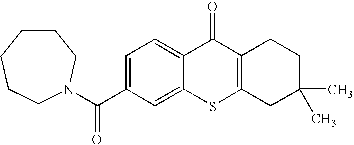 | 76.9% | 5.12 | 370 |

### EXAMPLE 3-8

### 6-(1-Azepanylcarbonyl)-3-ethyl-1,2,3,4-tetrahydro-9H-thioxanthen-9-one

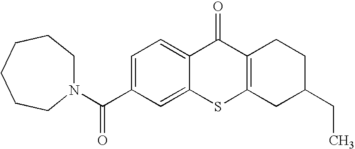

50.0 mg (0.17 mmol) of 3-ethyl-9-oxo-2,3,4,9-tetrahydro-1H-thioxanthene-6-carboxylic acid, 246 mg (0.23 mmol) of PS-carbodiumide (0.94 mmol/g) and 26.6 mg (0.20 mmol) of 1-hydroxy-1H-benzotriazole are shaken in 3 ml of dichloromethane at 25° C. under argon for 30 min. Then 13 μl (0.12 mmol) of azepane are added and the resulting suspension is shaken at 25° C. for 24 h. 165 mg (0.57 mmol) of PS-trisamine (3.5 mmol/g) are added, and shaking is continued for 8 h. After filtration, the organic phase is mixed with sodium carbonate solution and filtered through an Extrelut NT1 cartridge. The crude product is purified by chromatography on silica gel (0.04-0.063 nm) with cyclohexane/ethyl acetate 3:1 as mobile phase.

Yield: 41.6 mg (97.4%).

R_{f} (cyclohexane/ethyl acetate 3:1)=0.77.

MS (EI): 370 (M+H).

HPLC, retention time=4.98 min.

¹H-NMR (200 MHz, CDCl₃): δ=0.99 (t, 3H), 1.20-1.75 (m, 11H), 1.79-1.94 (m, 1 H), 1.97-2.12 (m, 1H), 2.33-2.64 (m, 2H), 2.65-2.81 (m, 1H), 2.84-3.02 (m, 1H), 3.26-3.40 (m, 2H), 3.70 (dd, 2H), 7.43 (dd, 1H), 7.52 (d, 1H), 8.52 (d, 1H).

### EXAMPLE 3-9

### 3-Ethyl-N-isopropyl-N-methyl-9-oxo-2,3,4,9-tetrahydro-1H-thioxanthene-6-carboxamide

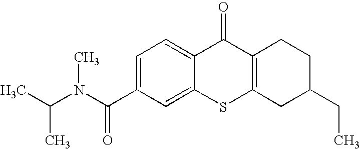

50.0 mg (0.17 mmol) of 3-ethyl-9-oxo-2,3,4,9-tetrahydro-1H-thioxanthene-6-carboxylic acid, 246 mg (0.23 mmol) of PS-carbodiimide (0.94 mmol/g) and 26.6 mg (0.20 mmol) of 1-hydroxy-1H-benzotriazole are shaken in 2 ml of dichloromethane at 25° C. under argon for 10 min. After addition of 12 μl (0.12 mmol) of N-isopropyl-N-methylamine, the resulting suspension is shaken at 25° C. for 24 h. 165 mg (0.57 mmol) of PS-trisamine (3.5 mmol/g) are added, and shaking is continued for 16 h. After filtration, the organic phase is mixed with aqueous sodium carbonate solution and filtered through an Extrelut NT1 cartridge. Removal of the solvent by distillation under reduced pressure and chromatography of the residue on silica gel (0.04-0.063 mm) with cyclohexane/ethyl acetate 2:1 as mobile phase affords the desired product.

Yield: 19.3 mg (48.6%).

MS (EI): 344 (M+H).

HPLC, retention time=4.76 min.

¹H-NMR (200 MHz, CDCl₃): δ=0.99 (t, 3H), 1.16, (d, 3H), 1.24 (d, 3H), 1.33-1.52 (m, 3H), 1.61-1.83 (m, 1H), 1.96-2.13 (m, 1H), 2.33-2.64 (m, 2H), 2.65-3.02 (m, 5H), 3.77-3.97 and 4.86-5.08 (m, 1H), 7.42 (br. d, 1H), 7.52 (br. s, 1H), 8.52 (d, 1H).

The carboxamides in Table 2 were obtained by the process described for Example 3-8 and 3-9.

TABLE 2

| | | | Retention | Mass |
|---|---|---|---|---|
| Ex. No. | Structure | Yield | time (min) | (M + H)⁺ |
| 3-10 | 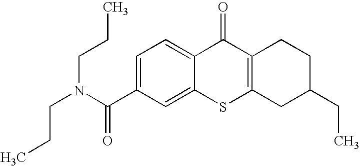 | 35.1% | 5.46 | 372 |
| 3-11 | 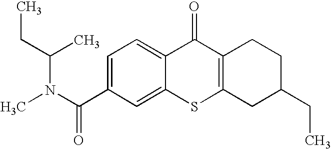 | 51.5% | 5.18 | 358 |
| 3-12 | 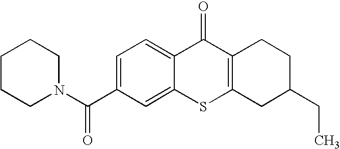 | 45.2% | 5.08 | 356 |
| 3-13 | 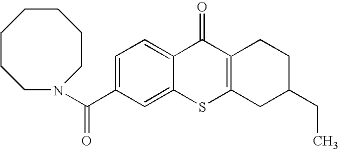 | 96.5% | 5.08 (LC-MS Method 1) | 384 |
| 3-14 | 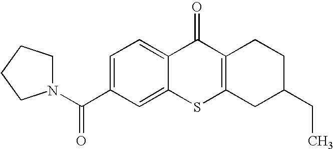 | 99.3% | 3.14 (LC-MS Method 3) | 342 |
| 3-15 | 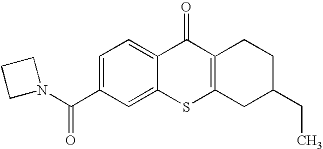 | 86.2% | 2.99 (LC-MS Method 3) | 328 |
| 3-16 | 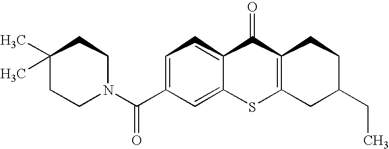 | 38.7% | 3.66 (LC-MS Method 3) | 384 |
| 3-17 | 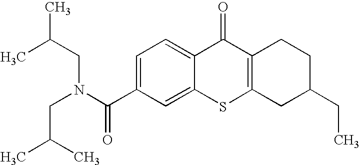 | 45.5% | 5.51 (LC-MS Method 2) | 400 |
| 3-18 | 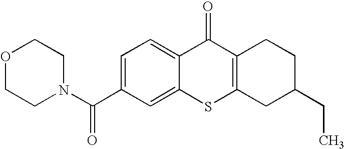 | 70.9% | 2.89 (LC-MS Method 3) | 358 |
| 3-19 | 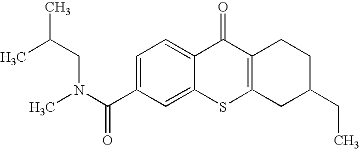 | 51.1% | 5.25 | 358 |
| 3-20 | 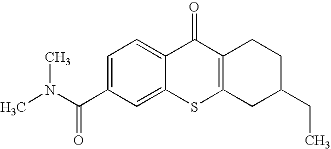 | 59.6% | 2.95 (LC-MS Method 3) | 316 |

### EXAMPLE 3-21

### 3-Ethyl-6-[(2-methoxyethyl)(methyl)amino]-1,2,3,4-tetrahydro-9H-thio-xanthen-9-one

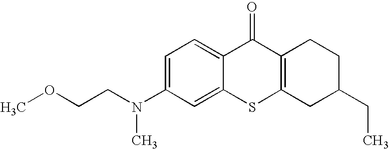

28.9 mg (0.03 mmol) of tris(dibenzylideneacetone)dipalladium, 58.9 mg (0.09 mmol) of (+/−)-2,2-bis(diphenylphosphino)-1,1-binaphthyl and 425 mg (4.42 mmol) of sodium tert-butoxide are added to 1020 mg (3.16 mmol) of 6-bromo-3-ethyl-1,2,3,4-tetrahydro-9H-thioxanthen-9-one in a flame-dried flask. The flask is evacuated and then flushed with argon for 10 min. 0.38 ml (3.79 mmol) of N-(2-methoxyethyl)methylamine and 5 ml of toluene are added, and the resulting suspension is stirred at 80° C. for 3 h. The reaction mixture is purified by chromatography on silica gel (0.04-0.063 mm) with cyclohexane/ethyl acetate 5:1/4:1/3:1/2:1 as mobile phase.

Yield: 477 mg (45.6%).

R_{f}(cyclohexane/ethyl acetate 3:1)=0.13.

MS (EI): 332 (M+H).

HPLC, retention time=5.05 min.

¹H-NMR (200 MHz, CDCl₃): δ=0.98 (t, 3H), 1.26-1.49 (m, 3H), 1.59-1.79 (m, 1H), 1.93-2.09 (m, 1H), 2.24-2.55 (m, 2H), 2.56-2.72 (m, 1H), 2.81-2.98 (m, 1H), 3.08 (s, 3H), 3.36 (s, 3H), 3.56-3.62 (m, 4H), 6.57 (d, 1H), 6.85 (dd, 1H), 8.31 (d, 1H).

### EXAMPLE 3-22

### 3-Ethyl-6-(1-piperidinyl)-1,2,3,4-tetrahydro-9H-thioxanthen-9-one

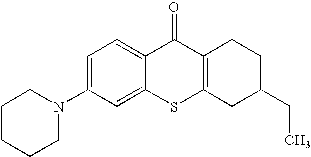

28.3 mg (0.03 mmol) of tris(dibenzylideneacetone)dipalladium, 58.9 mg (0.09 mmol) of (+/−)-2,2-bis(diphenylphosphino)-1,1-binaphthyl and 416 mg (4.33 mmol) of sodium tert-butoxide are added to 1000 mg (3.09 mmol) of 6-bromo-3-ethyl-1,2,3,4-tetrahydro-9H-thioxanthen-9-one in a flame-dried flask. The flask is evacuated and then flushed with argon for 10 min. 0.37 ml (3.71 mmol) of piperidine and 20 ml of toluene are added, and the resulting suspension is stirred at 80° C. for 3 h. Dilution with 10 ml of diethyl ether, filtration, removal of the solvent and subsequent recrystallization of the residue from tert-butyl methyl ether affords some of the desired product. The major fraction is obtained by chromatography of the filtrate from the recrystallization on silica gel (0.04-0.063 mm) with cyclohexane/ethyl acetate 7:1/5:1 as mobile phase.

Yield: 719 mg (69.6%)

R_{f} (cyclohexane/ethyl acetate 3:1)=0.40.

MS (DCI): 328 (M+H).

HPLC, retention time=5.43 min.

¹H-NMR (200 MHz, CDCl₃): δ=0.98 (t, 3H), 1.22-1.49 (m, 3H), 1.59-1.79 (m, 7H), 1.83-2.09 (m, 1H), 2.25-2.55 (m, 2H), 2.56-2.73 (m, 1H), 2.81-2.9 (m, 1H), 3.26-3.43 (m, 4H), 6.75 (d, 1H), 7.02 (dd, 1H), 8.32 (d, 1H).

### EXAMPLE 3-23

### 3-Ethyl-7-fluoro-6-(1-piperidinyl)-1,2,3,4-tetrahydro-9H-thioxanthen-9-one

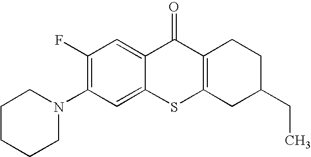

309.9 mg (0.54 mmol) of tris(dibenzylideneacetone)dipalladium, 321.7 mg (0.09 mmol) of 2-(di-t-butylphosphino)biphenyl and 777.1 mg (8.09 mmol) of sodium tert-butoxide are added to 1600 mg (5.39 mmol) of 6-chloro-3-ethyl-7-fluoro-1,2,3,4-tetrahydro-9H-thioxanthen-9-one in a flame-dried flask. The flask is evacuated and then flushed with argon for 10 min. 0.37 ml (3.71 mmol) of piperidine and 10 ml of toluene (degassed) are added, and the resulting suspension is stirred at 100° C. for 16 h. Since reaction was still incomplete, a further 30.9 mg of catalyst, 32.1 mg of ligand, 77.7 mg of base and 0.04 ml of piperidine are added, and the mixture is stirred at 100° C. for 24 h. The reaction mixture is mixed with 3 ml of ethyl acetate, filtered through an Extrelut cartridge and then freed of solvent. The residue is purified by chromatography on silica gel (0.04-0.063 mm) with cyclohexane/ethyl acetate 15:1/10:1 as mobile phase.

Yield: 545 mg (29.2%).

R_{f} (cyclohexane/diethyl ether 10:1)=0.17.

MS (EI): 346 (M+H).

HPLC, retention time=5.80 min.

¹H-NMR (200 MHz, CDCl₃): δ=0.95 (t, 3H), 1.29-1.50 (m, 3H), 1.57-1.83 (m, 7H), 1.85-2.10 (m, 1H), 2.29-2.59 (m, 2H), 2.60-2.75 (m, 1H), 2.81-2.98 (m, 1H), 3.18 (dd, 4H), 6.87 (d, 1H), 8.07 (d, 1H).

### EXAMPLE 3-24

### 6-(1,5-Dioxa-9-azaspiro[5.5]undec-9-yl)-3-ethyl-1,2,3,4-tetrahydro-9H-thioxanthen-9-one

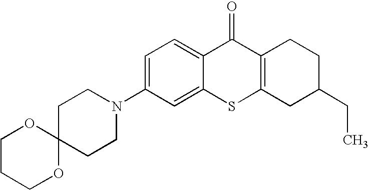

2.8 mg (0.003 mmol) of tris(dibenzylideneacetone)dipalladium, 5.9 mg (0.009 mmol) of (+/−)-2,2-bis(diphenylphosphino)-1,1-binaphthyl and 41.6 mg (0.43 mmol) of sodium tert-butoxide are added to 100 mg (0.31 mmol) of 6-bromo-3-ethyl-1,2,3,4-tetrahydro-9H-thioxanthen-9-one in a flame-dried flask. The flask is evacuated and then flushed with argon for 10 min. 59.6 mg (0.37 mmol) of 1,5-dioxa-9-azaspiro[5.5]undecane and 1 ml of toluene are added, and the resulting suspension is stirred at 80° C. for 5 h. Purification of the reaction mixture by chromatography on silica gel (0.04-0.063 mm) with cyclohexane/ethyl acetate 5:1/3:1/1:1 as mobile phase affords the required product.

Yield: 83 mg (67.5%).

R_{f} (cyclohexane/ethyl acetate 1:1)=0.38.

MS (LC-MS): 400 (M+H).

HPLC, retention time=5.03 min (LC-MS Method 1).

¹H-NMR (200 MHz, CDCl₃): δ=0.98 (t, 3H), 1.27-1.49 (m, 3H), 1.64-1.84 (m, 3H), 1.93-2.07 (m, 5H), 2.26-2.57 (m, 2H), 2.58-2.73 (m, 1H), 2.81-2.98 (m, 1H), 3.43 (dd, 4H), 3.95 (dd, 4H), 6.77 (d, 1H), 7.03 (dd, 1H), 8.33 (d, 1H).

The compounds in Table 3 were obtained by the processes described for Examples 3-21 to 3-24.

TABLE 3

| | | | Retention | Mass |
|---|---|---|---|---|
| Ex. No. | Structure | Yield | time (min) | (M + H)⁺ |
| 3-25 | 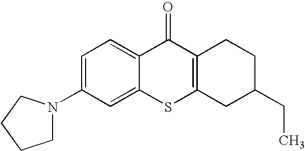 | 18.8% | 5.4 | 314 |
| 3-26 | 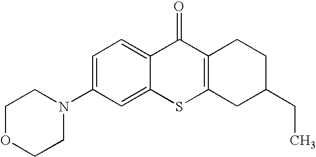 | 29.6% | 5.06 | 330 |
| 3-27 | 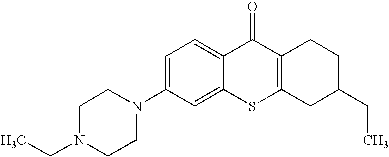 | 39.6% | 4.22 | 357 |
| 3-28 | 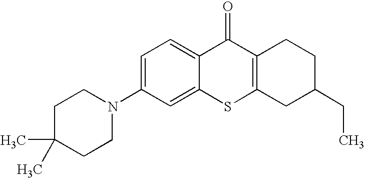 | 18.7% | 5.73 | 356 |
| 3-29 | 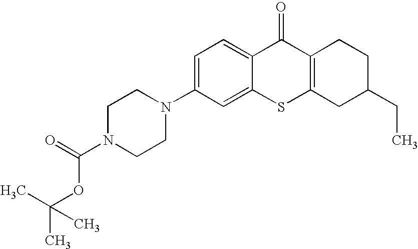 | 21.8% | 5.55 | 429 |
| 3-30 | 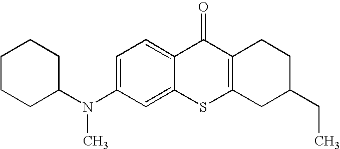 | 29.5% | 6.05 (LC-MS Method 1) | 356 |
| 3-31 | 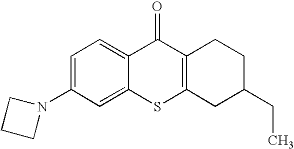 | 88.9% | 5.29 | 300 |
| 3-32 | 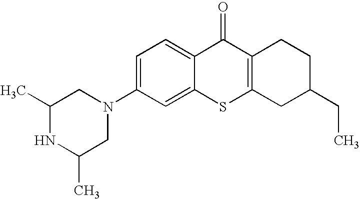 | 48.1% | 4.28 | 357 |
| 3-33 | 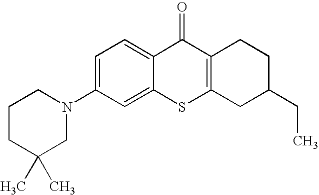 | 22.3% | 6.51 | 356 |
| 3-34 | 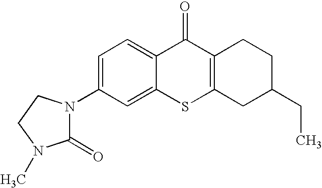 | 70.5% | 4.78 | 343 |
| 3-35 | 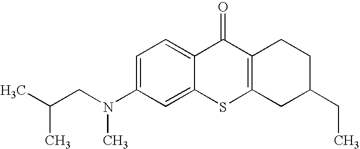 | 18.3% | 5.68 (LC-MS Method 2) | 330 |
| 3-36 | 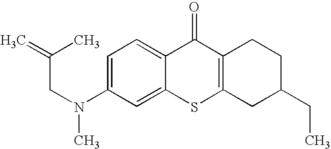 | 31.6% | 5.42 (LC-MS Method 2) | 328 |
| 3-37 | 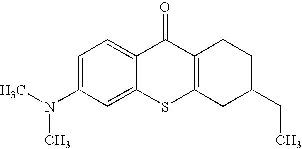 | 58.5% | 5.35 | 287 |
| 3-38 | 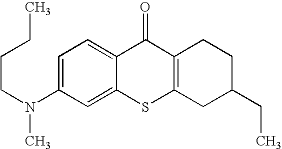 | 20.5% | 6.17 | 329 |
| 3-39 | 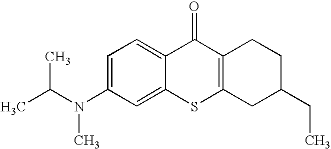 | 9.4% | 5.41 (LC-MS Method 2) | 316 |

### EXAMPLE 3-40

### 1-(3-Ethyl-9-oxo-2,3,4,9-tetrahydro-1H-thioxanthen-6-yl)-4-piperidinone

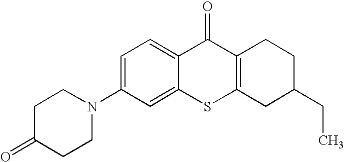

At 0° C., 0.50 ml of triflouroacetic acid (40% in H₂O) is added dropwise to a solution of 65 mg (0.16 mmol) of 6-(1,5-dioxa-9-azaspiro[5.5]undec-9-yl)-3-ethyl-1,2,3,4-tetrahydro-9H-thioxanthen-9-one in 5 ml of dichloromethane. The mixture is stirred at 25° C. for 48 h and then 10 ml of sodium hydroxide solution (1 N) are added. After extraction of the organic phase with dichloromethane, the combined organic phases are dried over sodium sulfate. Filtration, removal of the solvent under weak vacuum and chromatography of the residue on silica gel (0.04-0.063 mm) with cyclo-hexane/diethyl ether 1:4 as mobile phase affords the desired product.

Yield: 27 mg (48.6%).

R_{f} (cyclohexane/diethyl ether 1:5)=0.17.

MS (EI): 342 (M+H).

HPLC, retention time=4.69 min.

¹H-NMR (200 MHz, CDCl₃): δ=0.99 (t, 3H), 1.30-1.51 (m, 3H), 1.59-1.81 (m, 1H), 1.95-2.12 (m, 1H), 2.26-2.78 (m, 3H), overlapped by 2.60 (dd, 4H), 2.81-2.99 (m, 1H), 3.77 (dd, 4H), 6.81 (d, 1H), 7.05 (dd, 1H), 8.39 (d, 1H).

### EXAMPLE 3-41

### 3-Ethyl-6-[methyl(phenyl)amino]-1,2,3,4-tetrahydro-9H-thioxanthen-9-one

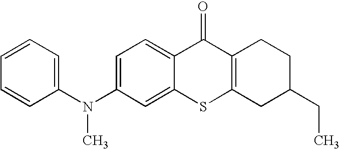

2.9 mg (0.005 mmol) of tris(dibenzylideneacetone)dipalladium, 3.1 mg (0.01 mmol) of 2-(di-t-butylphosphino)biphenyl and 17.3 mg (0.18 mmol) of sodium tert-butoxide are added to 41.7 mg (0.13 mmol) of 6-bromo-3-ethyl-1,2,3,4-tetrahydro-9H-thioxanthen-9-one in a flame-dried flask. The flask is evacuated and then flushed with argon. 0.02 ml (0.15 mmol) of N-methylaniline and 0.3 ml of toluene are added, and the resulting suspension is stirred at 80° C. for 24 h. The reaction mixture is diluted with 10 ml of ethyl acetate, filtered through Celite and then purified by preparative HPLC (RP-C18, acetonitrile/H₂O gradient).

Yield: 32.1 mg (71.2%).

R_{f} (cyclohexane/ethyl acetate 5:1)=0.28.

MS (EI): 350 (M+H).

HPLC, retention time=5.89 min.

¹H-NMR (200 MHz, CDCl₃): δ=0.98 (t, 3H), 1.22-1.49 (m, 3H), 1.59-1.78 (m, 1H), 1.93-2.09 (m, 1H), 2.25-2.72 (m, 3H), 2.81-2.98 (m, 1H), 3.38 (s, 3H), 6.70 (d, 1H), 6.86 (dd, 1H), 7.18-7.31 (m, 3H), 7.36-7.48 (m, 2H), 8.26 (d, 1H).

The compounds in Table 4 were obtained by the process described for Example 3-41.

TABLE 4

| | | | Retention | Mass |
|---|---|---|---|---|
| Ex. No. | Structure | Yield | time (min) | (M + H)⁺ |
| 3-42 | 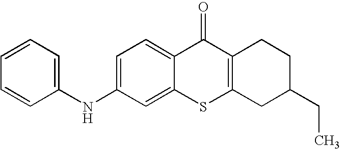 | 43.5% | 5.61 | 336 |
| 3-43 | 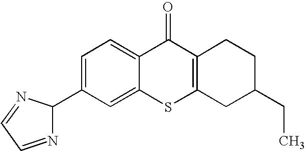 | 8.1% | 5.00 (LC-MS Method 1) | 311 |

### EXAMPLE 3-44

### 3-[(3-Ethyl-9-oxo-2,3,4,9-tetrahydro-1-thioxanthen-6-yl)oxy]-4-methylbenzonitrile

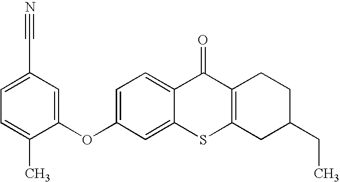

6.8 mg (0.007 mmol) of tris(dibenzylideneacetone)dipalladium, 3.4 mg (0.006 mmol) of 2-(di-t-butylphosphino)biphenyl and 78.8 mg (0.37 mmol) of potassium phosphate are added to 60 mg (0.19 mmol) of 6-bromo-3-ethyl-1,2,3,4-tetrahydro-9H-thioxanthen-9-one in a flame-dried flask. The flask is evacuated and then flushed with argon. After addition of 30 mg (0.22 mmol) of 2-hydroxy-4-methylbenzonitrile and 1.0 ml of toluene (degassed), the resulting suspension is stirred at 100° C. for 24 h. The reaction mixture is diluted with 3 ml of dichloromethane and, after addition of 0.5 ml of sodium hydroxide solution (1 N), filtered through an NT1-Extrelut cartridge. Removal of the solvent under reduced pressure and chromatography of the residue on silica gel (0.04-0.063 mm) with cyclohexane/diethyl ether 5:1 as mobile phase leads to the desired product.

Yield: 29.1 mg (41.8%).

R_{f} (cyclohexane/ethyl acetate 5:1)=0.33.

MS (EI): 376 (M+H).

HPLC, retention time=5.78 min.

¹H-NMR (200 MHz, CDCl₃): δ=0.99 (t, 3H), 1.21-1.50 (m, 3H), 1.60-1.79 (m, 1H), 1.96-2.12 (m, 1H), 2.30 (s, 3H), 2.34-2.77 (m, 3H), 2.82-3.00 (m, 1H), 6.88 (d, 1H), 7.03 (dd, 1H), 7.20-7.31 (m, 1H, overlapped by CHCl₃ signal), 7.35 (br. d, 1H), 7.46 (dd, 1H), 8.50 (d, 1H).

The compounds in Table 5 were obtained by the process described for Example 3-44.

TABLE 5

| | | | Retention | Mass |
|---|---|---|---|---|
| Ex. No. | Structure | Yield | time (min) | (M + H)⁺ |
| 3-45 | 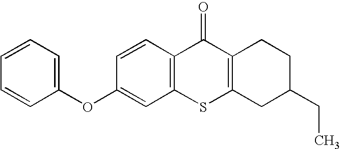 | 49.3% | 5.74 (LC-MS Method 2) | 337 |
| 3-46 | 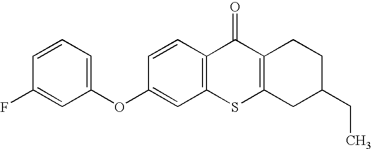 | 45.5% | 5.76 (LC-MS Method 2) | 355 |
| 3-47 | 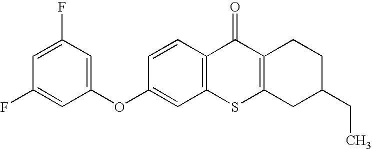 | 39.6% | 5.86 (LC-MS Method 1) | 373 |
| 3-48 | 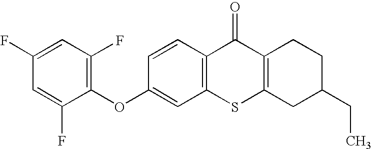 | 10.5% | 5.58 (LC-MS Method 2) | 391 |

### EXAMPLE 3-49

### N-(3-Ethyl-9-oxo-2,3,4,9-tetrahydro-1H-thioxanthen-6-yl)propanamide

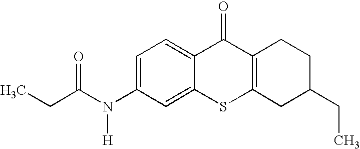

30 μl (0.30 mmol) of propionyl chloride are added dropwise to a suspension of 70 mg (0.27 mmol) of 6-amino-3-ethyl-1,2,3,4-tetrahydro-9H-thioxanthen-9-one in 3.5 ml of dichloromethane. After 10 min, 40 μl (0.30 mmol) of triethylamine are added, and the mixture is stirred under reflux for 4 h. After cooling, the reaction solution is poured into 10 ml of ice-water and, after thawing, part of the product is filtered off as solid. The latter is purified by recrystallization from acetonitrile. The filtrate is neutralized with sat. aqueous sodium carbonate solution. The solid obtained after extraction of the aqueous phase with dichloromethane and removal of the solvent is recrystallized from acetonitrile.

Yield: 71 mg (78.4%).

R_{f} (cyclohexane/ethyl acetate 1:2=0.58.

MS (EI): 315 (M).

HPLC, retention time=4.89 min.

¹H-NMR (200 MHz, DMSO-d₆): δ=0.94 (t, 3H), 1.09 (t, 3H), 1.21-1.46 (m, 3H), 1.51-1.75 (m, 1H), 1.87-2.03 (m, 1H), 2.26-2.47 (m, 4H), 2.62-2.82 (m, 2H), 7.53 (dd, 1H), 8.17 (d, 1H), 8.23 (d, 1H), 10.4 (s, 1H).

The compounds in Table 6 were obtained by the process described for Example 3-49.

TABLE 6

| | | | Retention | Mass |
|---|---|---|---|---|
| Ex. No. | Structure | Yield | time (min) | (M + H)⁺ |
| 3-50 | 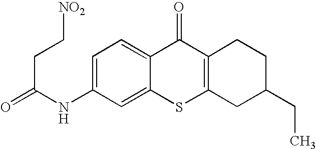 | 7.8% | 4.4 | 361 |
| 3-51 | 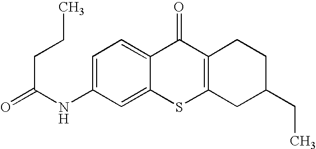 | 85.6% | 5.09 | 330 |
| 3-52 | 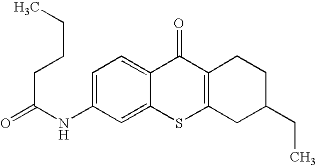 | 95.6% | 5.3 | 344 |
| 3-53 | 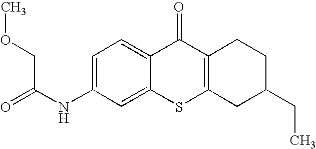 | 89.3% | 4.72 | 332 |

The compounds in Table 7 were obtained by the process described for Example 1-10:

TABLE 7

| | | | Retention | Mass |
|---|---|---|---|---|
| Ex. No. | Structure | Yield | time (min) | (M + H)⁺ |
| 3-54 | 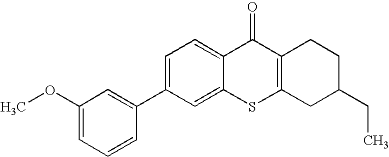 | 74.1% | 5.87 | 351 |
| 3-55 | 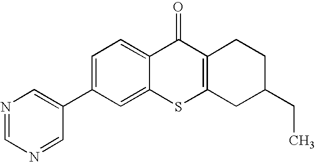 | 56.3% | 4.85 | 323 |
| 3-56 | 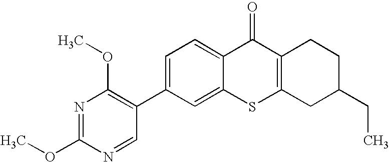 | 53.9% | 5.02 | 383 |
| 3-57 | 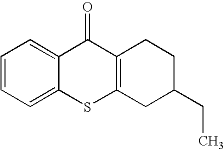 | 35.3% (by product) | 5.19 | 245 |
| 3-58 | 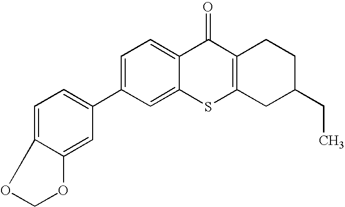 | 27.3% | 5.63 | 365 |
| 3-59 | 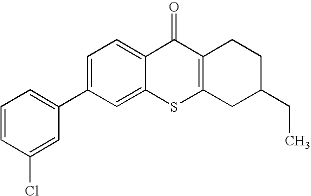 | 75.2% | 6.49 | 355 |
| 3-60 | 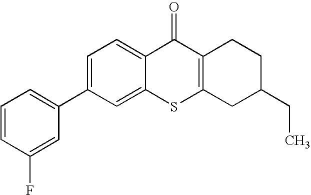 | 48.9% | 6.2 | 339 |
| 3-61 | 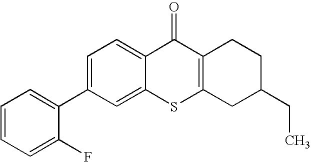 | 50.2% | 6.19 | 339 |
| 3-62 | 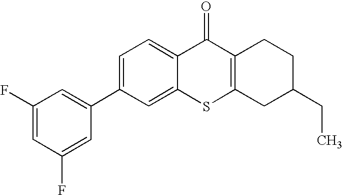 | 58.8% | 6.28 | 357 |
| 3-63 | 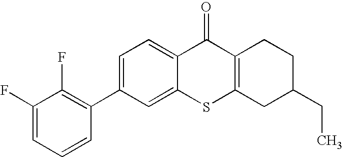 | 88.7% | 6.08 | 357 |
| 3-64 | 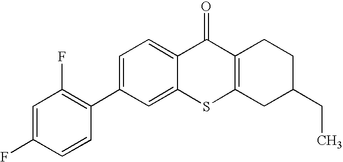 | 84.7% | 6.01 | 356 |
| 3-65 | 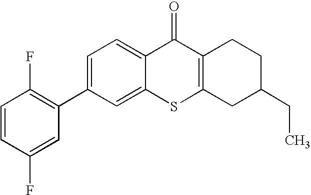 | 94.3% | 5.99 | 356 |
| 3-66 | 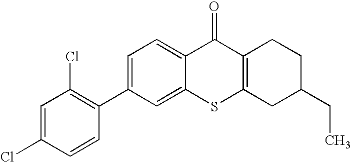 | 71.3% | 6.62 | 388 |
| 3-67 | 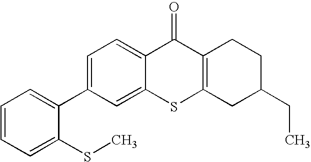 | 69.8% | 6.05 | 367 |
| 3-68 | 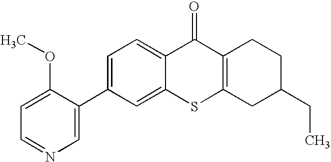 | 18.4% | 4.34 | 352 |

### EXAMPLE 3-69

### N,3,3-Trimethyl-9-oxo-N-(2,2,2-trifluoroethyl)-2,3,4,9-tetrahydro-1-thioxanthene-6-carboxamide

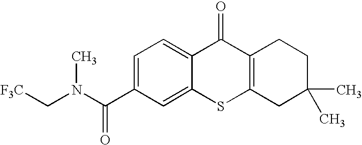

0.70 g (2.43 mmol) of 3,3-dimethyl-9-oxo-2,3,4,9-tetrahydro-1H-thioxanthene-6-carboxylic acid and 3.22 g (7.28 mmol) of 1-benzotriazolyloxytris(dimethylamino)-phosphonium hexafluorophosphate are stirred in 14 ml of THF at 25° C. under argon for 15 min. Firstly 0.85 ml (6.07 mmol) of triethylamine and, after a further 15 min, 1.10 g (9.71 mmol) of N-(2-trifluoroethyl)methylamine are added dropwise to the solution, which is then stirred at 25° C. for 24 h. Dilution with dichloromethane is followed by addition of citric acid (5% strength in water). The phases are separated and the organic phase is washed with aqueous sodium bicarbonate solution. After the combined organic phases have been dried over sodium sulfate and filtered, the solvent is distilled out under reduced pressure. The residue is purified by preparative

HPLC (RP-C18, acetonitrile/water gradient).

Yield: 838 mg (88.8%).

R_{f} (cyclohexane/ethyl acetate 2/1)=0.20.

MS (DCI): 384 (M+H).

HPLC, retention time=4.86 min.

¹H-NMR (200 MHz, CDCl₃): δ=1.04 (s, 6H), 1.64 (t, 2H), 2.49 (br. s, 2H), 2.72 (t, 2H), 3.11 (br. s, 3H), 3.66-4.35 (m, 2H), 7.47 (d 1H), 7.58 (s, 1H), 8.56 (d, 1H).

### EXAMPLE 3-70

### 7-Fluoro-N-isobutyl-N,3,3-trimethyl-9-oxo-2,3,4,9-tetrahydro-1H-thioxanthene-6-carboxamide

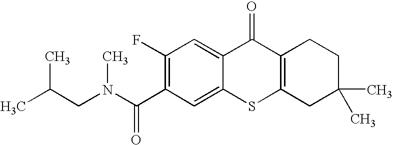

0.43 g (1.40 mmol) of 5-fluoro-3,3-dimethyl-9-oxo-2,3,4,9-tetrahydro-1H-thioxanthene-6-carboxylic acid and 1.86 g (4.21 mmol) of 1-benzotriazolyloxytris(dimethylamino)phosphonium hexafluorophosphate are stirred in 7 ml of THF at 25° C. under argon for 15 min. Firstly 0.49 ml (3.51 mmol) of triethylamine and, after a further 15 min, 0.49 g (5.61 mmol) of N-methylisobutylamine are added dropwise to the solution, which is then stirred at 25° C. for 24 h. Dilution with dichloromethane is followed by addition of citric acid (5% strength in water). The phases are separated and the organic phase is washed with aqueous sodium bicarbonate solution. After the combined organic phases have been dried over sodium sulfate and filtered, the solvent is distilled out under reduced pressure. The residue is purified by preparative HPLC (RP-C18, acetonitfile/water gradient).

Yield: 541.4 mg (85.6%).

R_{f} (cyclohexane/ethyl acetate 2/1)=0.45.

MS (EI): 376 (M).

HPLC, retention time=5.17 min.

¹H-NMR (200 MHz, CDCl₃): δ=0.76 (d, 3H), 1.00 (d, 3H), 1.04 (s, 6H), 1.64 (t, 2H), 1.84-2.19 (m, 1H), 2.49 (br. s, 2H), 2.72 (t, 2H), 2.89 and 3.10 (2s, 3H), 3.00 and 3.41 (2d, 2H), 7.53 (dd, 1H), 8.21 (dd, 1H).

The carboxamides in Table 8 are obtained in analogy to the processes described for Examples 3-1, 3-6, 3-7, 3-69 and 3-70.

TABLE 8

| | | | Retention | Mass |
|---|---|---|---|---|
| Ex. No. | Structure | Yield | time (min) | (M + H)⁺ |
| 3-71 | 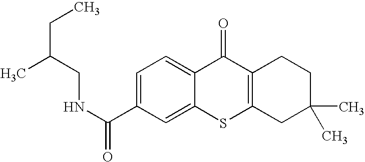 | 64.5% | 5.15 | 357 (M) |
| 3-72 | 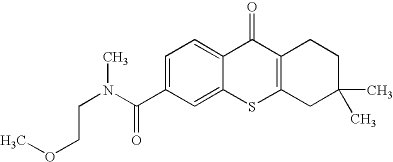 | 52.9% | 2.63 (LC-MS Method 3) | 360 |
| 3-73 | 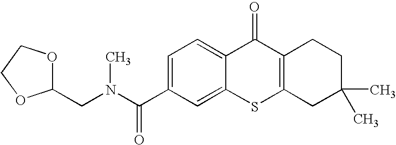 | 54.7% | 4.54 | 388 |
| 3-74 | 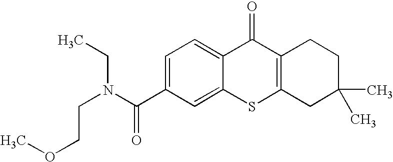 | 53.7% | 4.69 | 374 |
| 3-75 | 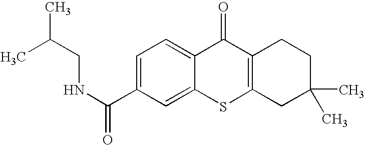 | 91.6% | 4.67 (LC-MS Method 3) | 344 |
| 3-76 | 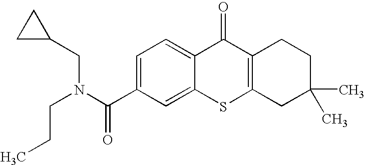 | 51.3% | 5.21 | 384 |
| 3-77 | 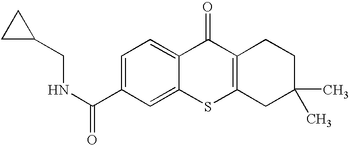 | 88.1% | 4.51 | 342 |
| 3-78 | 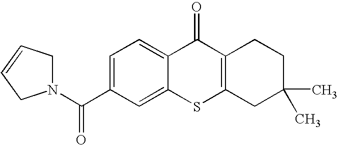 | 92.8% | 4.26 (LC-MS Method 1) | 340 |
| 3-79 | 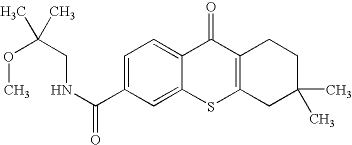 | 86.6% | 4.62 (LC-MS Method 1) | 374 |
| 3-80 | 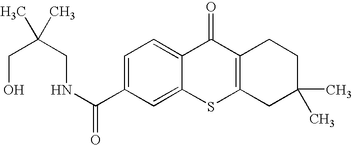 | 83.9% | 4.26 (LC-MS Method 1) | 374 |
| 3-81 | 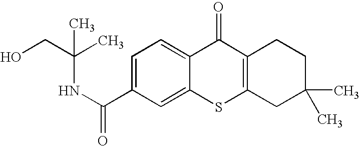 | 60.5% | 4.13 (LC-MS Method 1) | 360 |
| 3-82 | 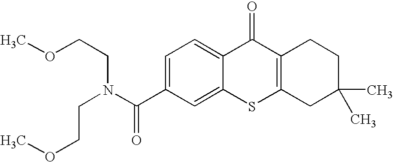 | 94.5% | 4.67 | 404 |
| 3-83 | 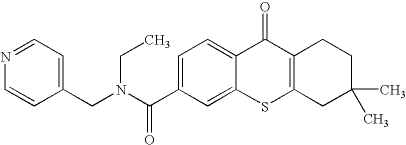 | 11.1% | 4.21 | 407 |
| 3-84 | 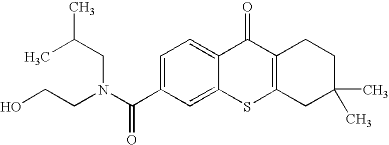 | 22.8% | 4.20 (LC-MS Method 1) | 388 |
| 3-85 | 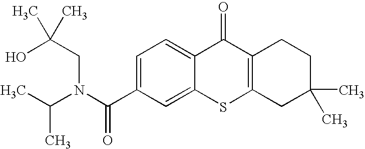 | 22.5% | 4.60 (LC-MS Method 1) | 401 (M) |
| 3-86 | 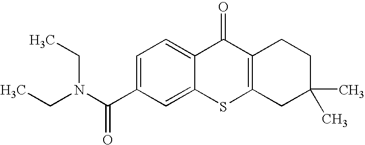 | 62.3% | 4.95 | 344 |
| 3-87 | 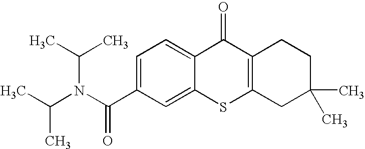 | 16.5% | 3.12 (LC-MS Method 3) | 372 |

The carboxamides in Table 9 were obtained in analogy to the processes described for Examples 3-8 and 3-9.

TABLE 9

| | | | Retention | Mass |
|---|---|---|---|---|
| Ex. No. | Structure | Yield | time (min) | (M + H)⁺ |
| 3-88 | 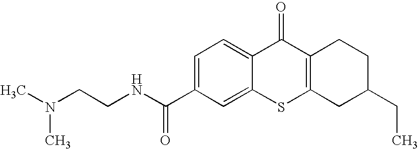 | 44.9% | 1.86 (LC-MS Method 3) | 359 |
| 3-89 | 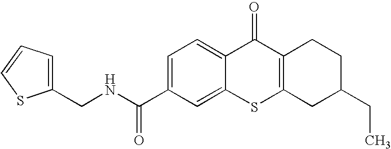 | 13.5% | 4.72 (LC-MS Method 2) | 384 |

### EXAMPLE 3-90

### N-Ethyl-N-(2-hydroxy-2-methylpropyl)-3,3-dimnethyl-9-oxo-2,3,4,9-tetrahydro-1H-thioxarthene-6-carboxamide

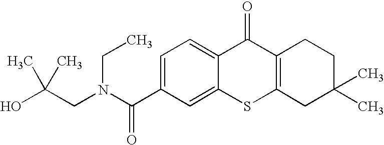

50 μl (0.46 mmol) of N-methylmorpho line, 35 mg (0.23 mmol) of N-ethylamino-2-methyl-2-propanol hydrochloride and 34 mg (0.25 mmol) of 1-hydroxy-1H-benzotriazole hydrate are added to a stirred suspension of 60 mg (0.21 mmol) of 3,3-dimethyl-9-oxo-2,3,4,9-tetrahydro-1H-thioxanthene-6-carboxylic acid in 10 ml of dichloromethane. The mixture is cooled to 0° C., and then 48 mg (0.25 mmol) of N-(3-dimethylaminopropyl)-N-ethylcarbodiimde hydrochloride are added. The cooling bath is removed and the mixture is stirred at 25° C. for 24 h. The solvent is distilled out under reduced pressure, and the resulting residue is purified by preparative HPLC (RP-C18, acetonitrile/water gradient). Pure carboxamide is subsequently obtained by a second chromatography of the prepurified product obtained in this way on silica gel (0.04-0.063 mm) (dichloromethane/methanol 30:1, 20:1, 10:1, 5:1, 1:1 as mobile phase).

Yield: 33.2 mg (41.2%).

R_{f} (CH₂Cl₂/MeOH 10/1)=0.55.

LC-MS (Method 1): 388 (M+H).

LC-MS, retention time=4.10 min.

¹H-NMR (200 MHz, CDCl₃): δ=1.04 (s, 6H), 1.08 (t, 3H), 1.33 (s, 6H), 1.64 (t, 2H), 2.49 (s, 2H), 2.73 (t, 2H), 3.37 (q, 2H), 3.58 (s, 2H), 4.06 (s, 1H), 7.47 (dd, 1H), 7.54 (br. s, 1H), 8.55 (d, 1H).

The carboxamides in Table 10 are obtained in analogy to the process described for Example 3-90.

TABLE 10

| | | | Retention | Mass |
|---|---|---|---|---|
| Ex. No. | Structure | Yield | time (min) | (M + H)⁺ |
| 3-91 | 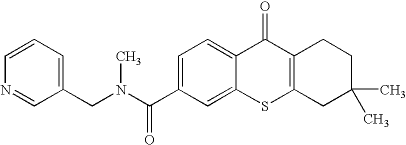 | 77.5% | 3.41 (LC-MS Method 2) | 393 |
| 3-92 | 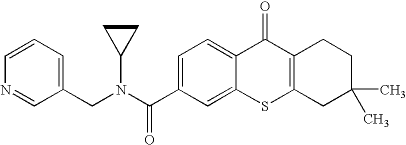 | 70.3% | 4.32 | 419 |
| 3-93 | 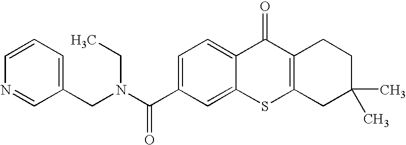 | 83.3 | 4.23 | 407 |

### EXAMPLE 3-94

### N-Ethyl-N-isobutyl-3,3-dimethyl-9-oxo-2,3,4,9-tetrahydro-1H-thioxanthene-6-carboxamide

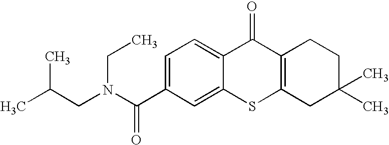

0.127 g (5.03 mmol) of sodium hydride is added to a solution of 0.576 g (1.68 mmol) of N-isobutyl-3,3-dimethyl-9-oxo-2,3,4,9-tetrahydro-1H-thioxanthene-6-carboxamide in 19 ml of THF at 0° C. under argon, and the mixture is stirred at 0° C. for 15 min. Then 0.67 ml (8.4 mmol) of iodoethane is added, and the cooling bath is removed. After stirring at 25° C. for 2 h, 2 ml of water are cautiously added, and dilution with ethyl acetate is followed by filtration through an Extrelut NT3 cartridge. The solvent is distilled off under reduced pressure, and the resulting residue is purified by preparative HPLC (RP-C 18, acetonitrile/water gradient).

Yield: 275.9 mg (43.6%).

R_{f} (cyclohexane/ethyl acetate 3/1)=0.23.

LC-MS (Method 2): 371 (M+H).

HPLC, retention time=5.00 min.

### EXAMPLE 3-95

### N-(Cyclopropylmethyl)-N-ethyl-3,3-dimethyl-9-oxo-2,3,4,9-tetrahydro-1H-thioxanthene-6-carboxamide

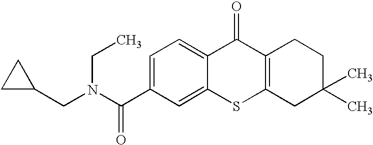

32.4 mg (1.28 mmol) of sodium hydride are added to a solution of 146 mg (0.43 mmol) of N-(cyclopropylmethyl)-3,3-dimethyl-9-oxo-2,3,4,9-tetrahydro-1H-thioxanthene-6-carboxamide in 2.8 ml of THF at 0° C. under argon, and the mixture is stirred at 0° C. for 15 min. Then 0.17 ml (2.1 mmol) of iodoethane is added, and the cooling bath is removed. After stirring at 25° C. for 1 h, 2 ml of water are cautiously added, and dilution with ethyl acetate is followed by filtration through an Extrelut NT3 cartridge. The solvent is distilled out under reduced pressure and the resulting residue is purified by preparative HPLC (RP-C18, acetonitrile/water gradient).

Yield: 275.9 mg (43.6%).

R_{f} (cyclohexane/ethyl acetate 1/1)=0.55.

LC-MS (Method 3): 370 (M+H).

LC-MS, retention time=3.02 min.

The compounds in Table 11 are obtained in analogy to the processes described for Examples 3-21 to 3-24.

TABLE 11

| | | | Retention | Mass |
|---|---|---|---|---|
| Ex. No. | Structure | Yield | time (min) | (M + H)⁺ |
| 3-96 | 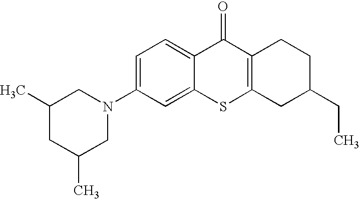 | 18.1% | 6.17 (LC-MS Method 2) | 356 |
| 3-97 | 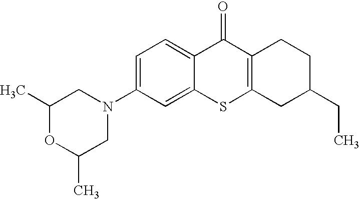 | 64.6% | 5.42 | 357 |
| 3-98 | 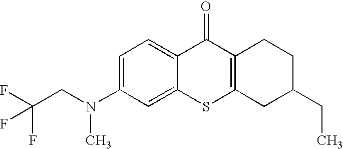 | 6.2 | 5.09 (LC-MS Method 2) | 356 |
| 3-99 | 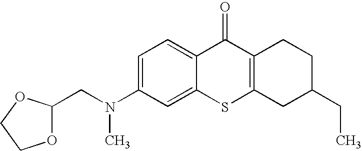 | 28.5 | 4.82 (LC-MS Method 2) | 360 |
| 3-100 | 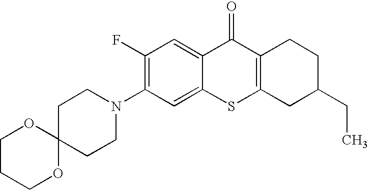 | 39.1 | 3.86 (LC-MS Method 3) | 418 |
| 3-101 | 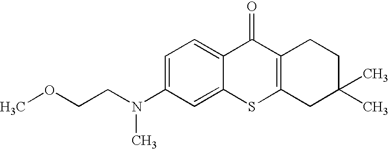 | 58.9 | 5.03 | 332 |
| 3-102 | 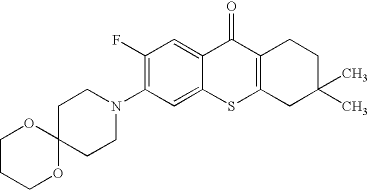 | 14.2 | 5.29 (LC-MS Method 1) | 418 |

The compounds in Table 12 are obtained from the corresponding ketals in analogy to the process described for

### EXAMPLE 3-40.

TABLE 12

| | | | Retention | Mass |
|---|---|---|---|---|
| Ex. No. | Structure | Yield | time (min) | (M + H)⁺ |
| 3-103 | 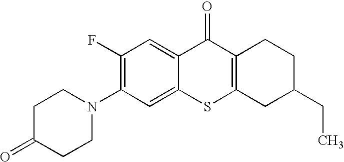 | 65.5% | 4.79 | 360 |
| 3-104 | 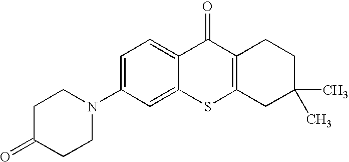 | 89.4% | 2.75 (LC-MS Method 3) | 342 |
| 3-105 | 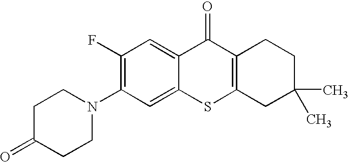 | 82.6% | 4.69 | 360 |

### EXAMPLE 3-106

### N-(3,3-Dimethyl-9-oxo-2,3,4,9-tetrahydro-1H-thioxanthen-6-yl)propanamide

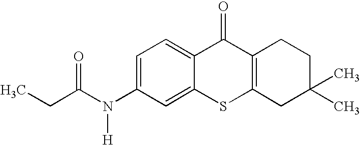

0.55 ml (6.36 mmol) of propionyl chloride is added dropwise to a suspension of 1.50 g (5.78 mmol) of 6-amino-3,3-dimethyl-1,2,3,4-tetrahydro-9H-thioxanthen-9-one in 75 ml of dichloromethane. After 10 min, 0.89 ml (6.36 mmol) of triethylamine is added, and the mixture stirred under reflux for 1 h. A further 50 μl (0.6 mmol) of propionyl chloride and 80 μl (0.6 mmol) of triethylamine are added and then the mixture is stirred under reflux for 3 h. After cooling, the reaction solution is added to 40 ml of ice-water and, after thawing, part of the product is filtered off as solid. The filtrate is taken up in 30 ml of dichloromethane and added to 20 ml of ice-water. After thawing, further product is filtered off as solid.

Yield: 1.337 g (73.3%).

R_{f} (cyclohexane/ethyl acetate 1:2=0.64.

MS (EI): 316 (M+H).

HPLC, retention time=4.73 min.

¹H-NMR (200 MHz, DMSO-d₆): δ=0.98 (s, 6H), 1.09 (t, 3H), 1.56 (t, 2H), 2.39 (q, 2H), 2.46-2.59 (m, 4H), 7.54 (dd, 1H), 8.17 (d, 1H), 8.24 (d, 1H), 10.3 (s, 1H).

### EXAMPLE 3-107

### N-(3,3-Dimethyl-9-oxo-2,3,4,9-tetrahydro-1H-thioxanthen-6-yl)-N,N-dimethylglycinamide

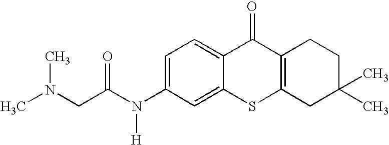

0.476 g (4.62 mmol) of N,N-dimethylglycine is added to a solution of 1.10 g (4.20 mmol) of 6-amino-3,3-dimethyl-1,2,3,4-tetrahydro-9H-thioxanthen-9-one and 2.39 g (6.30 mmol) of o-(7-azobenzotriazol-1-yl)-N,N,N′,N′-tetramethyluronium hexafluorophosphate in 11 ml of N,N-dimethylformamide under argon. The mixture is stirred at 60° C. for 9 h. After cooling, the reaction mixture is purified by preparative HPLC (RP-C18, acetonitrile/water gradient).

Yield: 71 mg (92.8%).

R_{f} (dichloromethane/methanol 20:1=0.35.

MS (EI): 345 (M+H).

HPLC, retention time=4.02 min.

¹H-NMR (200 MHz, DMSO-d₆): δ=0.98 (s, 6H), 1.57 (t, 2H), 2.39-2.61 (m, 4H), 2.88 (s, 6H), 4.17 (s, 2H), 7.59 (dd, 1H), 8.10 (d, 1H), 8.32 (d, 1H), 10.9 (s, 1H).

The compounds in Table 13 are obtained in analogy to the process described for Example 3-107.

TABELLE 13

| | | | Retention | Mass |
|---|---|---|---|---|
| Ex. No. | Structure | Yield | time (min) | (M + H)⁺ |
| 3-108 | 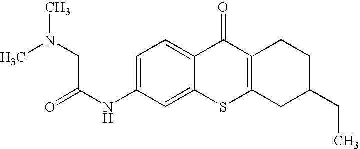 | 42.6% | 2.91 (LC-MS Method 2) | 345 |
| 3-109 | 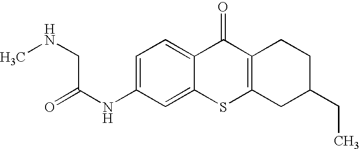 | 8.9% | 2.88 (LC-MS Method 2) | 331 |

### STARTING COMPOUNDS IV

### EXAMPLE IV-1

### N-(3-Ethyl-9-oxo-2,3,4,9-tetrahydro-1H-thioxanthen-6-yl)-1-butanesulfonamide

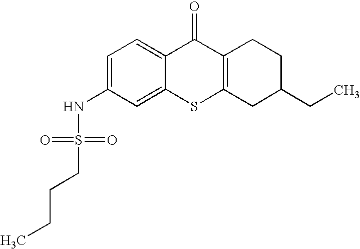

120 μl (1.45 mmol) of pyridine and a spatula tip of 4-dimethylaminopyridine are added to a solution of 75 mg (0.29 mmol) of 6-amino-3-ethyl-1,2,3,4-tetrahydro-9H-thioxanthen-9-one and 55 mg (0.35 mmol) of butanesulfonyl chloride in 2 ml of methylene chloride. The mixture is stirred at room temperature overnight. The reaction mixture is then diluted with 30 ml of methylene chloride and subsequently washed with water, 1N hydrochloric acid and saturated sodium chloride solution. The organic phase is dried over sodium sulfate and concentrated. The resulting residue is recrystallized from ethyl acetate. 15 mg (0.04 mmol, 13% yield) of the product are obtained as a pale yellow microcrystalline solid.

R_{f}: 0.23 (cyclohexane/ethyl acetate 2:1).

¹H-NMR (300 MHz, CDCl₃, δ/ppm): 8.45 (d, 1H), 7.40 (d, 1H), 7.14 (dd, 1H), 6.69 (s, 1H), 3.16 (m, 2H), 2.91 (m, 1H), 2.61 (dd, 1H), 2.58-2.34 (m, 2H), 2.02 (m, 1H), 1.82 (m, 2H), 1.71 (m, 1H), 1.48-1.32 (m, 5H), 0.99 (t, 3H), 0.89 (t, 3H).

MS (ESI): 380.2 ([M+H]⁺).

### EXAMPLE IV-2

### N-(3-Ethyl-9-oxo-2,3,4,9-tetrahydro-1-thioxanthen-6-yl)-3-pyridinesulfonamide

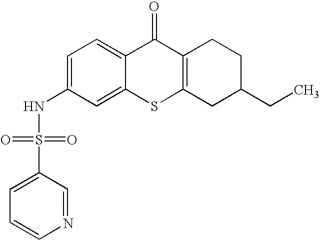

60 mg (0.15 mmol, 51% yield) of the product are obtained as a pale yellow microcrystalline solid from 75 mg (0.29 mmol) of 6-amino-3-ethyl-1,2,3,4-tetrahydro-9H-thioxanthen-9-one and 75 mg (0.35 mmol) of 3-pyridylsulfonyl chloride in analogy to the synthesis of N-(3-ethyl-9-oxo-2,3,4,9-tetrahydro-1H-thioxanthen-6-yl)-1-butanesulfonamide.

R_{f}: 0.21 (cyclohexane/ethyl acetate 2:1).

MS (ESI): 401.1 ([M+H]⁺).

### EXAMPLE IV-3

### N-(3-Ethyl-9-oxo-2,3,4,9-tetrahydro-1-thioxanthen-6-yl)-3-methylbenzenesulfonamide

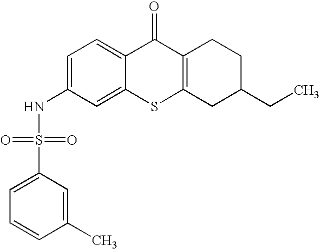

14 mg (0.03 mmol, 9% yield) of the product are obtained as a colorless microcrystalline solid from 100 mg (0.39 mmol) of 6-amino-3-ethyl-1,2,3,4-tetrahydro-9H-thioxanthen-9-one and 87 mg (0.46 mmol) of m-tolylsulfonyl chloride in analogy to the synthesis of N-(3-ethyl-9-oxo-2,3,4,9-tetrahydro-1H-thioxanthen-6-yl)-1-butanesulfonamide.

R_{f}: 0.24 (cyclohexane/ethyl acetate 2:1).

MS (ESI): 414 ([M+H]⁺).

### EXAMPLE IV-4

### 2-Cyano-N-(3-ethyl-9-oxo-2,3,4,9-tetrahydro-1H-thioxanthen-6-yl)-benzenesulfonamide

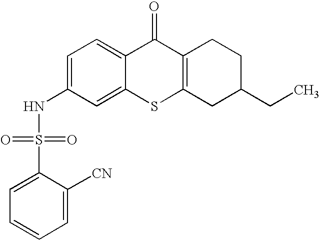

60 mg (0.14 mmol, 49% yield) of the product are obtained as a pale yellow microcrystalline solid from 75 mg (0.29 mmol) of 6-amino-3-ethyl-1,2,3,4-tetrahydro-9H-thioxanthen-9-one and 71 mg (0.35 mmol) of 2-cyanophenylsulfonyl chloride in analogy to the synthesis of N-(3-ethyl-9-oxo-2,3,4,9-tetrahydro-1H-thioxanthen-6-yl)-1-butanesulfonamide.

R_{f}: 0.18 (cyclohexane/ethyl acetate 2:1).

MS (ESI): 425.0 ([M+H]⁺).

### EXAMPLE IV-5

### 3-Cyano-N-(3-ethyl-9-oxo-2,3,4,9-tetrahydro-1-thioxanthen-6-yl)-benzenesulfonamide

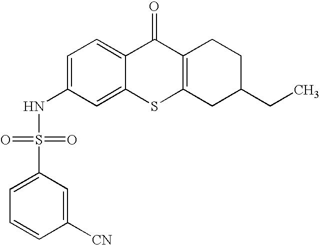

20 mg (0.05 mmol, 16% yield) of the product are obtained as a pale yellow microcrystalline solid from 75 mg (0.29 mmol) of 6-amino-3-ethyl-1,2,3,4-tetrahydro-9H-thioxanthen-9-one and 71 mg (0.35 mmol) of 3-cyanophenylsulfonyl chloride in analogy to the synthesis of N-(3-ethyl-9-oxo-2,3,4,9-tetrahydro-1H-thioxanthen-6-yl)-1-butanesulfonamide.

R_{f}: 0.18 (cyclohexane/ethyl acetate 2:1).

MS (ESI): 425.0 ([M+H]⁺).

### EXAMPLE IV-6

### 4-Cyano-N-(3-ethyl-9-oxo-2,3,4,9-tetrahydro-1H-thioxanthen-6-yl)-benzenesulfonamide

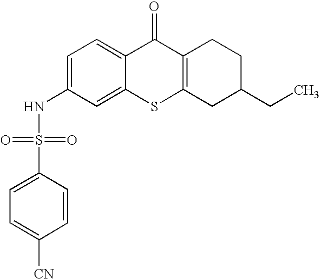

140 mg (0.33 mmol, 85% yield) of the product are obtained as a pale yellow microcrystalline solid from 100 mg (0.39 mmol) of 6-amino-3-ethyl-1,2,3,4-tetrahydro-9H-thioxanthen-9-one and 93 mg (0.46 mmol) of 4-cyanophenylsulfonyl chloride in analogy to the synthesis of N-(3-ethyl-9-oxo-2,3,4,9-tetrahydro-1H-thioxanthen-6-yl)-1-butanesulfonamide.

R_{f}: 0.18 (Cyclohexane/ethyl acetate 2:1).

MS (ESI): 425.0 ([M+H]⁺).

### EXAMPLE IV-7

### N-(3-Ethyl-9-oxo-2,3,4,9-tetrahydro-1H-thioxanthen-6-yl)-1-propanesulfonamide

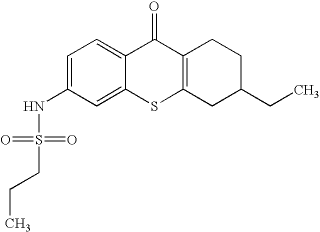

25 mg (0.07 mmol, 23% yield) of the product are obtained as a yellow microcrystalline solid from 75 mg (0.29 mmol) of 6-amino-3-ethyl-1,2,3,4-tetrahydro-9H-thioxanthen-9-one and 50 mg (0.35 rnmol) of n-propylsulfonyl chloride in analogy to the synthesis of N-(3-ethyl-9-oxo-2,3,4,9-tetrahydro-1H-thioxanthen-6-yl)-1-butanesulfonamide.

R_{f}: 0.19 (cyclohexane/ethyl acetate 2:1).

¹H-NMR (300 MHz, DMSO-d₆, δ/ppm): 10.49 (s, 1H), 8.25 (d, 1H), 7.38 (d, 1H), 7.33 (dd, 1H) 3.24 (t, 2H), 2.70 (m, 2H), 2.41 (t, 2H), 2.11 (t, 2H), 1.95 (m, 1H), 1.67 (m, 2H), 1.37 (m, 2H), 1.11 (1, 3H), 0.93 (t, 3H).

MS (ESI): 366.2 ([M+H]⁺).

### EXAMPLE IV-8

### N-(3-Ethyl-9-oxo-2,3,4,9-tetrahydro-1-thioxanthen-6-yl)cyclopropanesulfonamide

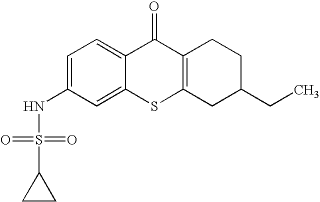

71 mg (0.19 mmol, 34% yield) of the product are obtained as a colorless microcrystalline solid from 150 mg (0.58 mmol) of 6-amino-3-ethyl-1,2,3,4-tetra-hydro-9H-thioxanthen-9-one and 97 mg (0.69 mmol) of cyclopropylsulfonyl chloride in analogy to the synthesis of N-(3-ethyl-9-oxo-2,3,4,9-tetrahydro-1H-thioxanthen-6-yl)-1-butanesulfonamide.

R_{f}: 0.25 (cyclohexane/ethyl acetate 2:1).

MS (ESI): 364 ([M+H]⁺).

### EXAMPLE IV-9

### N-(3-Ethyl-9-oxo-2,3,4,9-tetrahydro-1-thioxanthen-6-yl)-4-methoxybenzenesulfonamide

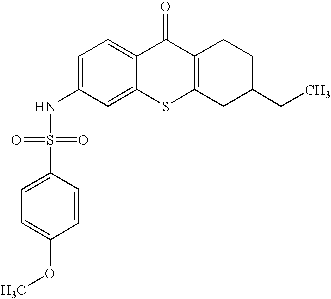

110 mg (0.26 mmol, 66% yield) of the product are obtained as a pale yellow microcrystalline solid from 100 mg (0.39 mmol) of 6-amino-3-ethyl-1,2,3,4-tetrahydro-9H-thioxanthen-9-one and 95 mg (0.46 mmol) of 4-methoxyphenylsulfonyl chloride in analogy to the synthesis of N-(3-ethyl-9-oxo-2,3,4,9-tetrahydro-1-thioxanthen-6-yl)-1-butanesulfonamide.

R_{f}: 0.15 (ethyl acetate/cyclohexane 2:1).

MS (ESI): 430 ([M+H]⁺).

### EXAMPLE IV-10

### 3-Chloro-N-(3-ethyl-9-oxo-2,3,4,9-tetrahydro-1-thioxanthen-6-yl)benzenesulfonamide

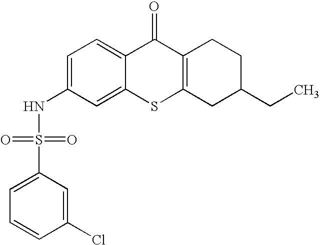

30 mg (0.07 mmol, 18% yield) of the product are obtained as a colorless microcrystalline solid from 100 mg (0.39 mmol) of 6-amino-3-ethyl-1,2,3,4-tetrahydro-9H-thioxanthen-9-one and 89 mg (0.42 mmol) of 3-chlorophenylsulfonyl chloride in analogy to the synthesis of N-(3-ethyl-9-oxo-2,3,4,9-tetrahydro-1H-thioxanthen-6-yl)-1-butanesulfonamide.

R_{f}: 0.33 (ethyl acetate/cyclohexane 2:1).

MS (ESI): 434 ([M+H]⁺).

### EXAMPLE IV-11

### N-(3-Ethyl-9-oxo-2,3,4,9-tetrahydro-1-thioxanthen-6-yl)-2-methylbenzenesulfonamide

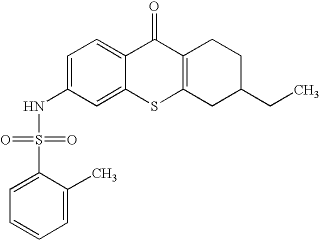

36 mg (0.09 mmol, 23% yield) of the product are obtained as a pale yellow microcrystalline solid from 100 mg (0.39 mmol) of 6-amino-3-ethyl-1,2,3,4-tetrahydro-9H-thioxanthen-9-one and 88 mg (0.46 mmol) of o-tolylsulfonyl chloride in analogy to the synthesis of N-(3-ethyl-9-oxo-2,3,4,9-tetrahydro-1H-thioxanthen-6-yl)-1-butanesulfonamide.

R_{f}: 0.16 (ethyl acetate/cyclohexane 2:1).

MS (ESI): 414.1 ([M+H]⁺).

### EXAMPLE IV-12

### N-(3-Ethyl-9-oxo-2,3,4,9-tetrahydro-1-thioxanthen-6-yl)-2,4-dimethylbenzenesulfonamide

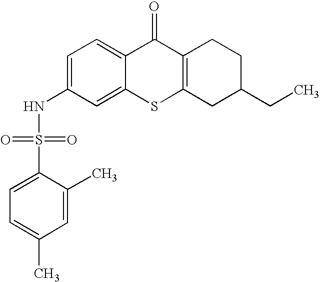

80 mg (0.19 mmol, 48% yield) of the product are obtained as a yellow microcrystalline solid from 100 mg (0.39 mmol) of 6-amino-3-ethyl-1,2,3,4-tetrahydro-9H-thioxanthen-9-one and 94 mg (0.46 mmol) of 2,4-dimethylphenylsulfonyl chloride in analogy to the synthesis of N-(3-ethyl-9-oxo-2,3,4,9-tetrahydro-1H-thioxanthen-6-yl)-1-butanesulfonamide.

R_{f}: 0.20 (ethyl acetate/cyclohexane 2:1).

MS (ESI): 428 ([M+H]⁺).

### EXAMPLE IV-13

### N-(3-Ethyl-9-oxo-2,3,4,9-tetrahydro-1H-thioxanthen-6-yl)-3-nitrobenzenesulfonamide

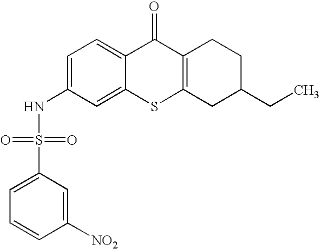

70 mg (0.15 mmol, 27% yield) of the product are obtained as a colorless microcrystalline solid from 150 mg (0.58 mmol) of 6-amino-3-ethyl-1,2,3,4-tetrahydro-9H-thioxanthen-9-one and 153 mg (0.69 minol) of 3-nitrophenylsulfonyl chloride in analogy to the synthesis of N-(3-ethyl-9-oxo-2,3,4,9-tetrahydro-1H-thioxanthen-6-yl)-1-butanesulfonamide.

R_{f}: 0.07 (ethyl acetate/cyclohexane 2:1).

MS (ESI): 444.9 ([M+H]⁺).

### EXAMPLE IV-14

### 5-Chloro-N-(3-ethyl-9-oxo-2,3,4,9-tetrahydro-1H-thioxanthen-6-yl)-2-thiophenesulfonamide

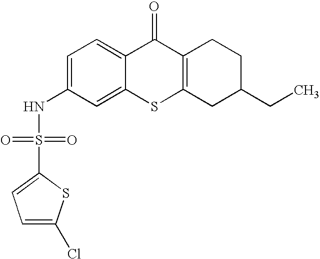

105 mg (0.24 mmol, 41% yield) of the product are obtained as a yellow microcrystalline solid from 150 mg (0.58 mmol) of 6-amino-3-ethyl-1,2,3,4-tetrahydro-9H-thioxanthen-9-one and 150 mg (0.69 mmol) of 5-chlorothiophenesulfonyl chloride in analogy to the synthesis of N-(3-ethyl-9-oxo-2,3,4,9-tetrahydro-1-thioxanthen-6-yl)-1-butanesulfonamide.

R_{f}: 0.33 (ethyl acetate/cyclohexane 2:1).

MS (ESI): 440 ([M+H]⁺).

### EXAMPLE IV-15

### N-(3-Ethyl-9-oxo-2,3,4,9-tetrahydro-1-thioxanthen-6-yl)-2-naphthalenesulfonamide

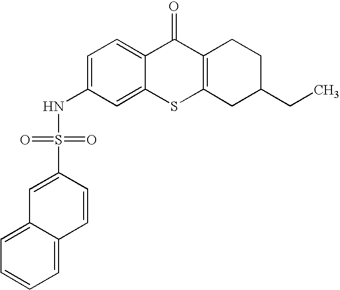

73 mg (0.16 mmol, 28% yield) of the product are obtained as a yellow microcrystalline solid from 150 mg (0.58 mmol) of 6-amino-3-ethyl-1,2,3,4-tetrahydro-9H-thioxanthen-9-one and 156 mg (0.69 mmol) of 2-naphthalenesulfonyl chloride in analogy to the synthesis of N-(3-ethyl-9-oxo-2,3,4,9-tetrahydro-1H-thioxanthen-6-yl)-1-butanesulfonamide

R_{f}: 0.41 (ethyl acetate/cyclohexane 2:1).

MS (ESI): 450 ([M+H]⁺).

### EXAMPLE IV-16

### N-(3-Ethyl-9-oxo-2,3,4,9-tetrahydro-1-thioxanthen-6-yl)-2-thiophenesulfonamide

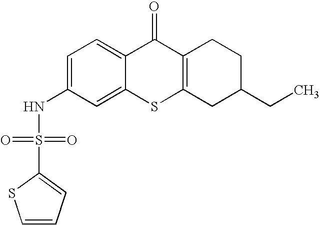

123 mg (0.30 mmol, 52% yield) of the product are obtained as a pale yellow solid from 150 mg (0.58 mmol) of 6-amino-3-ethyl-1,2,3,4-tetrahydro-9H-thioxanthen-9-one and 126 mg (0.69 mmol) of 2-thiophenesulfonyl chloride in analogy to the synthesis of N-(3-ethyl-9-oxo-2,3,4,9-tetrahydro-1H-thioxanthen-6-yl)-1-butanesulfonamide.

R_{f}: 0.36 (ethyl acetate/cyclohexane 2:1).

MS (ESI): 406 ([M+H]⁺).

### EXAMPLE IV-17

### 2-(1,3-Dioxo-1,3-dihydro-2H-isoindol-2-yl)-N-(3-ethyl-9-oxo-2,3,4,9-tetrahydro-1H-thioxanthen-6-yl)ethanesulfonamide

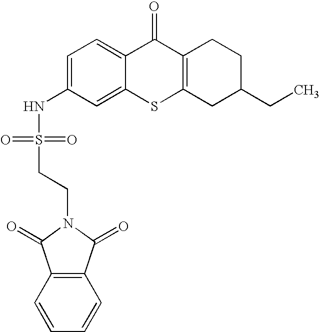

125 mg (0.25 mmol, 42% yield) of the product are obtained as a pale yellow solid from 150 mg (0.58 mmol) of 6-amino-3-ethyl-1,2,3,4-tetrahydro-9H-thioxanthen-9-one and 188 mg (0.69 mmol) of 2-(1,3-dioxo-1,3-dihydro-2H-isoindol-2-yl)ethanesulfonyl chloride in analogy to the synthesis of N-(3-ethyl-9-oxo-2,3,4,9-tetrahydro-1H-thioxanthen-6-yl)-1-butanesulfonamide.

R_{f}: 0.32 (ethyl acetate/cyclohexane 2:1).

MS (ESI): 497.1 ([M+H]⁺).

### EXEMPLARY EMBODIMENTS 4

### EXAMPLE 4-1

### 6-Amino-3-ethyl-1,2,3,4-tetrahydro-9H-thioxanthen-9-one

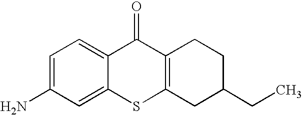

426 g of polyphosphoric acid are stirred at room temperature under argon for 15 minutes. They are then heated at about 150° C. for 5 minutes and allowed to cool, and 15.0 g (120 mmol) of 3-aminothiophenol and 21.6 g (109 mmol) of ethyl 4-ethyl-2-oxocyclohexanecarboxylate are cautiously added. The mixture is stirred at 90° C. for 2 hours and allowed to cool to room temperature. 430 ml of ice-water are added to the resulting red mixture and, after stirning for 30 minutes, it is extracted ten times with ethyl acetate. The combined organic phases are washed successively with water, saturated sodium bicarbonate solution, water and saturated sodium chloride solution and dried over sodium sulfate. After removal of the solvent, the crude product is mixed with methylene chloride, whereupon part of the product (5.46 g) precipitates as a yellow solid. The filtrate is purified by column chromatography (silica gel cyclohexane/ethyl acetate 40:1). The product fraction (6.18 g) is concentrated and dried in vacuo. A total of 11.64 g (44.9 mmol, 41% yield) of a yellow solid is obtained.

R_{f}: 0.17 (cyclohexane/ethyl acetate 2:1).

¹H-NMR (300 MHz, DMSO-d₆, δ/ppm): 7.98 (d, 1H), 6.72 (dd, 1H), 6.60 (d, 1H), 6.12 (s, 2H), 2.66 (m, 2H), 2.32 (m, 2H), 1.91 (m, 1H), 1.62 (m, 1H), 1.34 (m, 3H), 0.93 (t, 3H).

MS (EI): 259 (M⁺).

### EXAMPLE 4-2

### 3-Ethyl-6-(ethylamino)-1,2,3,4-tetrahydro-9H-thioxanthen-9-one

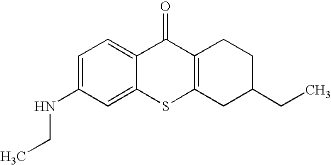

2.19 mg (57.8 mmol) of sodium borohydride are added to a solution of 1.5 g (5.8 mmol) of 6-amino-3-ethyl-1,2,3,4-tetrahydro-9H-thioxanthen-9-one in 40 ml of glacial acetic acid, and the mixture is stirred at room temperature for 2 hours. The solution is then diluted with 200 ml of water, and 2N sodium hydroxide solution is added until the pH is 9. Extraction with methylene chloride and drying on sodium sulfate are followed by evaporation to dryness. The crude product is purified by column chromatography (silica gel, methylene chloride/methanol 600:1-100:1). The product fraction is concentrated and dried in vacuo. 1.01 g (3.51 mmol, 60% yield) of a pale yellow solid are obtained. 6-(Diethylamino)-3-ethyl-1,2,3,4-tetrahydro-9H-thioxanthen-9-one is obtained as byproduct.

R_{f}: 0.74 (methylene chloride/methanol 20:1).

¹H-NMR (300 MHz, DMSO-d₆, δ/ppm): 7.99 (d, 1H), 6.76 (dd, 1H), 6.65 (t, 1H), 6.56 (d, 1H), 3.12 (dq, 2H), 2.67 (m, 2H), 2.34 (m, 2H), 1.92 (m, 1H), 1.60 (m, 1H), 1.32 (m, 3H), 1.18 (t, 3H), 0.94 (t, 3H).

MS (EI: 287 (M⁺).

### EXAMPLE 4-3

### 6-(Diethylamino)-3-ethyl-1,2,3,4-tetrahydro-9H-thioxanthen-9-one

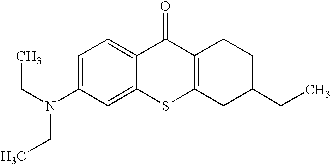

6-(Diethylamino)-3-ethyl-1,2,3,4-tetrahydro-9H-thioxanthen-9-one is obtained as byproduct in the synthesis of 3-ethyl-6-(ethylamino)-1,2,3,4-tetrahydro-9H-thioxanthen-9-one. 373 mg (1.18 mmol, 20% yield) of a pale yellow solid are isolated.

R_{f}: 0.37 (cyclohexane/ethyl acetate 20:1).

¹H-NMR (300 MHz, DMSO-d₆, δ/ppm): 8.07 (d, 1H), 6.89 (dd, 1H), 6.68 (d, 1H), 3.42 (q, 4H), 2.67 (m, 2H), 2.34 (m, 2H), 1.92 (m, 1H), 1.60 (m, 1H), 1.32 (m, 3H), 1.12 (t, 6H), 0.94 (t, 3H).

MS (EI): 315 (M⁺).

### EXAMPLE 4-4

### N-(3-Ethyl-9-oxo-2,3,4,9-tetrahydro-1H-thioxanthen-6-yl)-N-methyl-1-butanesulfonamide

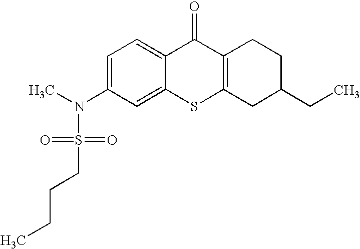

45 mg (0.32 mmol) of methyl iodide and 109 mg (0.79 mmol) of potassium carbonate are added to a solution of 30 mg (0.08 mmol) of N-(3-ethyl-9-oxo-2,3,4,9-tetrahydro-1H-thioxanthen-6-yl)-1-butanesulfonamide in 2 ml of acetone. The mixture is stirred under reflux overnight. The reaction mixture is then evaporated to dryness, and the residue is partitioned between methylene chloride and water. The organic phase is dried over sodium sulfate and concentrated. The resulting residue is fractionated by preparative HPLC (column: Kromasil 120 ODS-4HE, 10 μm, 250×20 mm; eluent: acetonitrile/water; flow rate: 25 ml/min; UV detection at 210 nm). 9 mg (0.02 mmol, 29% yield) of the product are obtained as a colorless crystalline solid.

R_{f}: 0.44 (cyclohexane/ethyl acetate 2:1).

MS (ESI): 394 ([M+H]⁺).

### EXAMPLE 4-5

### N-(3-Ethyl-9-oxo-2,3,4,9-tetrahydro-1-thioxanthen-6-yl)-N-methyi-3-pyridinesulfonamide

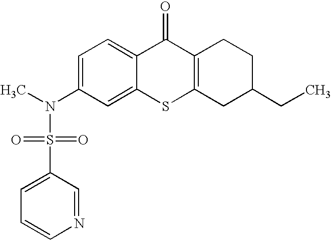

7 mg (0.02 mmol, 21% yield) of the product are obtained as a pale yellow microcrystalline solid from 30 mg (0.07 mmol) of N-(3-ethyl-9-oxo-2,3,4,9-tetrahydro-1H-thioxanthen-6-yl)-3-pyridinesulfonamide, 39 mg (0.28 mmol) of methyl iodide and 97 mg (0.70 mmol) of potassium carbonate in analogy to the synthesis of N-(3-ethyl-9-oxo-2,3,4,9-tetrahydro-1-thioxanthen-6-yl)-N-methyi-1-butanesulfonamide.

R_{f}: 0.38 (cyclohexane/ethyl acetate 2:1).

MS (ESI): 415.2 ([M+H]⁺).

### EXAMPLE 4-6

### N-(3-Ethyl-9-oxo-2,3,4,9-tetrahydro-1H-thioxanthen-6-yl)-N,3-dimethylbenzenesulfonamide

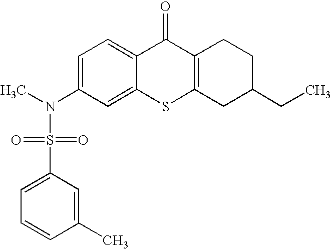

24 mg (0.06 mmol, 47% yield) of the product are obtained as a colorless microcrystalline solid from 50 mg (0.12 mmol) of N-(3-ethyl-9-oxo-2,3,4,9-tetrahydro-1H-thioxanthen-6-yl)-3-methylbenzenesulfonamide, 68 mg (0.48 mmol) of methyl iodide and 167 mg (1.21 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.46 (cyclohexane/ethyl acetate 20:1).

MS (ESI): 428.1 ([M+H]⁺).

### EXAMPLE 4-7

### 2-Cyano-N-(3-ethyl-9-oxo-2,3,4,9-tetrahydro-1H-thioxanthen-6-yl)-N-methylbenzenesulfonamide

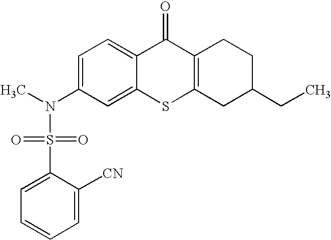

39 mg (0.09 mmol, 75% yield) of the product are obtained as a colorless microcrystalline solid from 50 mg (0.12 mmol) of 2-cyano-N-(3-ethyl-9-oxo-2,3,4,9-tetrahydro-1H-thioxanthen-6-yl)-benzenesulfonamide, 67 mg (0.47 mmol) of methyliodide and 98 mg (0.71 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.43 (cyclohexane/ethyl acetate 2:1).

¹H-NMR (300 MHz, CDCl₃, δ/ppm): 8.38 (d, 1H), 7.72 (m, 2H), 7.65 (m, 2H), 7.50 (d, 1H), 7.13 (dd, 1H), 3.48 (s, 3H), 2.89 (m, 1H), 2.70 (dd, 1H), 2.55-2.35 (m, 2H), 2.03 (m, 1H), 1.70 (m, 1H), 1.48-1.36 (m, 3H), 0.99 (t, 3H).

MS (ESI): 439.2 ([M+H]⁺).

### EXAMPLE 4-8

### 3-Cyano-N-(3-ethyl-9-oxo-2,3,4,9-tetrahydro-1-thioxanthen-6-yl)-N-methylbenzenesulfonamide

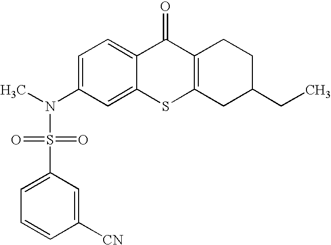

27 mg (0.06 mmol, 52% yield) of the product are obtained as a colorless microcrystalline solid from 50 mg (0.12 mmol) of 3-cyano-N-(3-ethyl-9-oxo-2,3,4,9-tetrahydro-1H-thioxanthen-6-yl)-benzenesulfonamide, 67 mg (0.47 mmol) of methyliodide and 98 mg (0.71 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.43 (cyclohexane/ethyl acetate 2:1).

¹H-NMR (300 MHz, CDCl₃, δ/ppm): 8.41 (d, 1H), 7.90 (m, 2H), 7.66 (m, 1H), 7.60 (d, 1H), 7.38 (d, 1H), 7.08 (dd, 1H), 3.27 (s, 3H), 2.91 (m, 1H), 2.72 (dd, 1H), 2.60-2.35 (m, 2H), 2.05 (m, 1H), 1.71 (m, 1H), 1.47-1.34 (m, 3H), 0.99 (t, 3H).

MS (ESI): 439.1 ([M+H]⁺).

### EXAMPLE 4-9

### 4-Cyano-N-(3-ethyl-9-oxo-2,3,4,9-tetrahydro-1-thioxanthen-6-yl)-N-methylbenzenesulfonamide

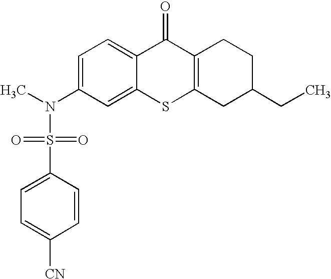

31 mg (0.07 mmol, 60% yield) of the product are obtained as a colorless amorphous solid from 50 mg (0.12 mmol) of 4-cyano-N-(3-ethyl-9-oxo-2,3,4,9-tetrahydro-1H-thioxanthen-6-yl)-benzenesulfonamide, 67 mg (0.47 mmol) of methyliodide and 163 mg (1.18 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.41 (cyclohexane/ethyl acetate 2:1).

¹H-NMR (300 MHz, CDCl₃, δ/ppm): 8.42 (d, 1H), 7.75 (d, 2H), 7.64 (d, 2H), 7.36 (d, 1H), 7.11 (dd, 1H), 3.27 (s, 3H), 2.91 (m, 1H), 2.72 (dd, 1H), 2.59-2.36 (m, 2H), 2.06 (m, 1H), 1.71 (m, 1H), 1.48-1.37 (m, 3H), 1.00 (t, 3H).

MS (ESI): 439 ([M+H]⁺).

### EXAMPLE 4-10

### N-(3-Ethyl-9-oxo-2,3,4,9-tetrahydro-1-thioxanthen-6-yl)-N-methyl-1-propanesulfonamide

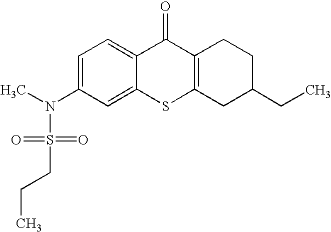

8 mg (0.02 mmol, 15% yield) of the product are obtained as a colorless microcrystalline solid from 50 mg (0.14 mmol) of N-(3-ethyl-9-oxo-2,3,4,9-tetrahydro-1H-thioxanthen-6-yl)-1-propanesulfonamide, 78 mg (0.55 mmol) of methyl iodide and 190 mg (1.38 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.29 (cyclohexane/ethyl acetate 2:1).

¹H-NMR (300 MHz, CDCl₃, δ/ppm): 8.48 (d, 1H), 7.56 (d, 1H), 7.45 (dd, 1H), 3.40 (s, 3H), 3.00 (m, 2H), 2.91 (m, 1H), 2.71 (dd, 1H), 2.58-2.35 (m, 2H), 2.06 (m, 1H), 1.82 (m, 2H), 1.71 (m, 1H), 1.48-1.36 (m, 3H), 1.02.(t, 3H), 0.99 (t, 3H).

MS (ESI): 380 ([M+H]⁺).

### EXAMPLE 4-11

### N-(3-Ethyl-9-oxo-2,3,4,9-tetrahydro-1-thioxanthen-6-yl)-N-methylcyclopropanesulfonamide

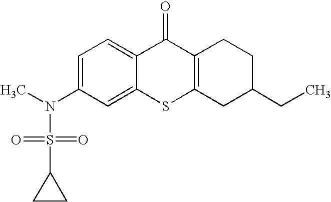

18 mg (0.05 mmol, 35% yield) of the product are obtained as a colorless amorphous solid from 50 mg (0.14 mmol) of N-(3-ethyl-9-oxo-2,3,4,9-tetrahydro-1H-thioxanthen-6-yl)cyclopropanesulfonamide, 78 mg (0.55 mmol) of methyl iodide and 190 mg (1.38 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.31 (cyclohexane/ethyl acetate 2:1).

MS (ESI): 378 ([M+H]⁺).

### EXAMPLE 4-12

### N-(3-Ethyl-9-oxo-2,3,4,9-tetrahydro-1-thioxanthen-6-yl)-4-methoxy-N-methylbenzenesulfonamide

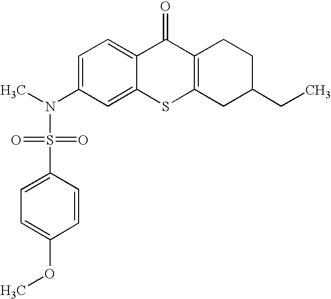

14 mg (0.03 mmol, 27% yield) of the product are obtained as a colorless amorphous solid from 50 mg (0.12 mmol) of N-(3-ethyl-9-oxo-2,3,4,9-tetrahydro-1H-thioxanthen-6-yl)-4-methoxybenzenesulfonamide, 67 mg (0.47 mmol) of methyl iodide and 160 mg (1.16 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.45 (cyclohexane/ethyl acetate 2:1).

¹H-NMR (300 MHz, CDCl₃, δ/ppm): 8.44 (d, 1H), 7.57 (t, 1H), 7.42 (m, 3H), 7.12 (dd, 1H), 3.26 (s, 3H), 2.92 (m, 1H), 2.72 (dd, 1H), 2.58-2.37 (m, 2H), 2.07 (m, 1H), 1.72 (m, 1H), 1.48-1.37 (m, 3H), 1.01 (t, 3H).

MS (ESI): 443.9 ([M+H]⁺).

### EXAMPLE 4-13

### 3-Chloro-N-(3-ethyl-9-oxo-2,3,4,9-tetrahydro-1-thioxanthen-6-yl)-N-methylbenzenesulfonamide

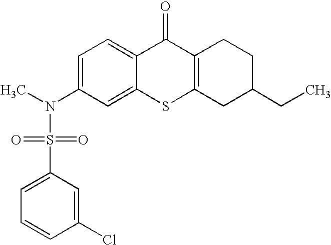

11 mg (0.02 mmol, 21% yield) of the product are obtained as a pale yellow microcrystalline solid from 50 mg (0.12 mmol) of 3-chloro-N-(3-ethyl-9-oxo-2,3,4,9-tetrahydro-1H-thioxanthen-6-yl)benzenesulfonamide, 65 mg (0.46 mmol) of methyl iodide and 159 mg (1.15 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.41 (cyclohexane/ethyl acetate 2:1).

MS (ESI): 448.0 ([M+H]⁺).

### EXAMPLE 4-14

### N-(3-Ethyl-9-oxo-2,3,4,9-tetrahydro-1H-thioxanthen-6-yl)-N,2-dimethylbenzenesulfonamide

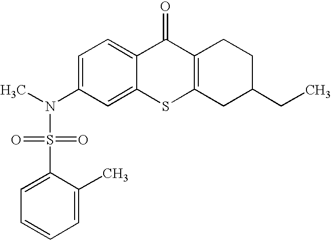

27 mg (0.06 mmol, 65% yield) of the product are obtained as a colorless amorphous solid from 40 mg (0.10 mmol) of N-(3-ethyl-9-oxo-2,3,4,9-tetrahydro-1H-thioxanthen-6-yl)-2-methylbenzenesulfonamide, 55 mg (0.39 rmol) of methyl iodide and 134 mg (0.97 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.48 (cyclohexane/ethyl acetate 2:1).

MS (ESI): 428.1 ([M+H]⁺).

### EXAMPLE 4-15

### N-(3-Ethyl-9-oxo-2,3,4,9-tetrahydro-1-thioxanthen-6-yl)-N,2,4-trimethylbenzenesulfonamide

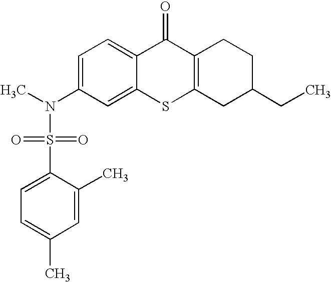

37 mg (0.08 mmol, 71% yield) of the product are obtained as a colorless amorphous solid from 50 mg (0.12 mmol) of N-(3-ethyl-9-oxo-2,3,4,9-tetrahydro-1H-thioxanthen-6-yl)-2,4-dimethylbenzenesulfonamide, 67mg (0.47rmmol) of methyl iodide and 162 mg (1.17 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.46 (cyclohexane/ethyl acetate 2:1).

MS (ESI): 442.4 ([M+H]⁺).

### EXAMPLE 4-16

### N-(3-Ethyl-9-oxo-2,3,4,9-tetrahydro-1H-thioxanthen-6-yl)-N-methyl-3-nitrobenzenesulfonamide

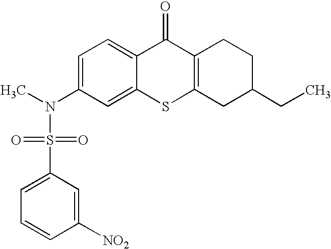

20 mg (0.04 mmol, 48% yield) of the product are obtained as a colorless amorphous solid from 40 mg (0.09 mmol) of N-(3-ethyl-9-oxo-2,3,4,9-tetrahydro-1H-thioxanthen-6-yl)-3-nitrobenzenesulfonamide, 51 mg (0.36 mmol) of methyl iodide and 124 mg (0.90 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.48 (cyclohexane/ethyl acetate 2:1).

MS (ESI): 459.2 ([M+H]⁺).

### EXAMPLE 4-17

### 5-Chloro-N-(3-ethyl-9-oxo-2,3,4,9-tetrahydro-1-thioxanthen-6-yl)-N-methyl-2-thiophenesulfonamide

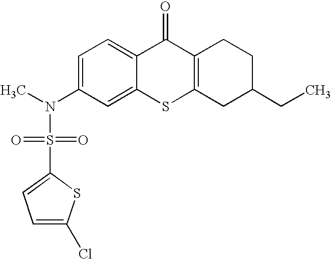

81 mg (0.18 mmol, 98% yield) of the product are obtained as a pale yellow microcrystalline solid from 80 mg (0.18 mmol) of 5-chloro-N-(3-ethyl-9-oxo-2,3,4,9-tetrahydro-1H-thioxanthen-6-yl)-2-thiophenesulfonamide, 104 mg (0.73 mmol) of methyl iodide and 252 mg (1.82 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.51 (cyclohexane/ethyl acetate 2:1).

MS (ESI): 454 ([M+H]⁺).

### EXAMPLE 4-18

### 3-Ethyl-6-(2-pyridinyl)-1,2,3,4-tetrahydro-9H-thioxanthen-9-one

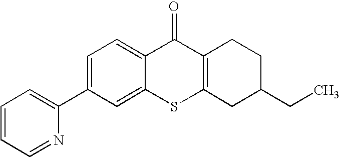

150 mg (0.46 mmol) of 6-bromo-3-ethyl-1,2,3,4-tetrahydro-9H-thioxanthen-9-one are introduced into 3 ml of dimethylformamide under an argon atmosphere. 141 mg (0.56 mmol) of bis(pinacolato)diboron, 137 mg (1.39 mmol) of potassium acetate and 10 mg (0.01 mmol) of dichloro[bis(diphenylphosphino)ferrocenyl]palladium(II) as catalyst are successively added to this solution, and the mixture is stirred at 70° C. for 4 h. Then 88 mg (0.56 mmol) of 2-bromopyridine, a further 10 mg of catalyst dissolved in 1 ml of dimethylformamide and 1 ml of 2M sodium carbonate solution are added to this solution, and the mixture is stirred at 70° C. overnight. After cooling, the solution is partitioned between ethyl acetate and water, and the organic phase is washed with water and dried over sodium sulfate. The residue obtained after concentration is fractionated by preparative HPLC (column: Kromasil 120 ODS-4 HE, 10 μm, 250×20 mm; eluent: acetonitrile/water; flow rate: 25 ml/min; UV detection at 210 nm). 21 mg (0.06 mmol, 14% yield) of the product are obtained as a colorless crystalline solid.

R_{f}: 0.62 (cyclohexane/ethyl acetate 2:1).

MS (ED): 321 (M⁺).

### EXAMPLE 4-19

### 3-Ethyl-6-(2-pyriridinyl)-1,2,3,4-tetrahydro-9H-thioxanthen-9-one

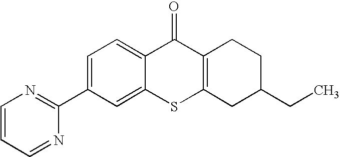

8 mg (0.02 mmol, 5% yield) of the product are obtained as a colorless mnicrocrystalline solid from 150 mg (0.46 mmol) of 6-bromo-3-ethyl-1,2,3,4-tetrahydro-9H-thioxanthen-9-one, 141 mg (0.56 mmol) of bis(pinacolato)diboron, 137 mg (1.39 mmol) of potassium acetate and 88 mg (0.56 mmol) of 2-bromopyrimidine in analogy to the synthesis of 3-ethyl-6-(2-pyridinyl)-1,2,3,4-tetrahydro-9H-thioxanthen-9-one.

R_{f}: 0.41 (cyclohexane/ethyl acetate 2:1).

MS (ESI): 323.2 ([M+H]⁺).

### EXAMPLE 4-20

### 2-(3-Ethyl-9-oxo-2,3,4,9-tetrahydro-1H-thioxanthen-6-yl)benzonitrile

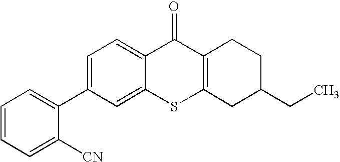

72 mg (0.21 mmol, 25% yield) of the product are obtained as a colorless microcrystalline solid from 150 mg (0.82 mmol) of 2-bromobenzonitrile, 251 mg (0.99 mmol) of bis(pinacolato)diboron, 243 mg (2.47 mmol) of potassium acetate and 320 mg (0.99 mmol) of 6-bromo-3-ethyl-1,2,3,4-tetrahydro-9H-thioxanthen-9-one in analogy to the synthesis of 3-ethyl-6-(2-pyridinyl)-1,2,3,4-tetrahydro-9H-thioxanthen-9-one.

R_{f}: 0.42 (cyclohexane/ethyl acetate 2:1).

¹H-NMR (300 MHz, DMSO-d₆, δ/ppm): 8.42 (d, 1H), 8.06 (d, 1H), 8.03 (d, 1H), 7.86 (m, 1H), 7.70 (m, 3H), 2.70 (m, 2H), 2.49 (m, 2H, overlapped by DMSO signal), 1.99 (m, 1H), 1.70 (m, 1H), 1.48-1.28 (m, 3H), 0.95 (t, 3H).

MS (EI): 345 (M⁺).

### EXAMPLE 4-21

### 3-Ethyl-6-(6-methyl-3-pyridinyl)-1,2,3,4-tetrahydro-9H-thioxanthen-9-one

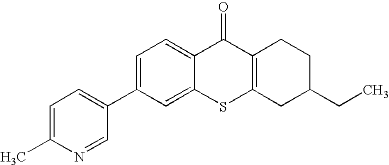

19 mg (0.06 mmol, 23% yield) of the product are obtained as a pale yellow amorphous solid from 80 mg (0.25 mmol) of 6-bromo-3-ethyl-1,2,3,4-tetrahydro-9H-thioxanthen-9-one, 76 mg (0.30 mmol) of bis(pinacolato)diboron, 73 mg (0.74 mmol) of potassium acetate and 52 mg (0.30 mmol) of 2-bromo-5-methylpyridine in analogy to the synthesis of 3-ethyl-6-(2-pyridinyl)-1,2,3,4-tetrahydro-9H-thioxanthen-9-one.

R_{f}: 0.34 (cyclohexane/ethyl acetate 2:1).

¹H-NMR (300 MHz, DMSO-d₆, δ/ppm): 8.58 (d, 1H), 8.45 (d, 1H), 8.41 (d, 1H), 8.25 (dd, 1H), 8.08 (d, 1H), 7.78 (dd, 1H), 2.69 (m, 2H), 2.44 (m, 2H, overlapped by DMSO signal), 2.38 (s, 3H), 1.98 (m, 1H), 1.68 (m, 1H), 1.46-1.25 (m, 3H), 0.94 (t, 3H).

MS (ED): 335 (M⁺).

### EXAMPLE 4-22

### 3-Ethyl-6-(6-chloro-3-pyridinyl)-1,2,3,4-tetrahydro-9H-thioxanthen-9-one

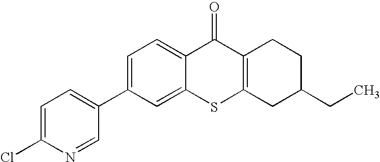

10 mg (0.03 mmol, 9% yield) of the product are obtained as a colorless amorphous solid from 100 mg (0.31 mmol) of 6-bromo-3-ethyl-1,2,3,4-tetrahydro-9H-thioxanthen-9-one, 86 mg (0.34 mmol) of bis(pinacolato)diboron, 91 mg (0.93 mmol) of potassium acetate and 71 mg (0.37 mmol) of 2-bromo-5-chloropyridine in analogy to the synthesis of 3-ethyl-6-(2-pyridinyl)-1,2,3,4-tetrahydro-9H-thioxanthen-9-one.

R_{f}: 0.38 (cyclohexane/ethyl acetate 2:1).

MS (ESI): 356 ([M+H]⁺).

### EXAMPLE 4-23

### 3-Ethyl-6-(2-methoxyphenyl)-1,2,3,4-tetrahydro-9H-thioxanthen-9-one

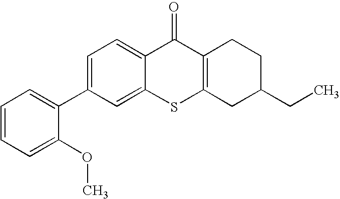

100 mg (0.31 mmol) of 6-bromo-3-ethyl-1,2,3,4-tetrahydro-9H-thioxanthen-9-one are introduced into 3 ml of dimethoxyethane under an argon atmosphere. 56 mg (0.37 mmol) of 2-methoxyphenylboronic acid, 10 mg (0.01 mmol) of dichloro[bis(triphenylphosphino)]palladium(II) as catalyst and 0.34 ml of 2M sodium carbonate solution are added successively to this solution, and the mixture is stirred at 90° C. for 2 h. After cooling, the solution is filtered through silica and washed with ethyl acetate. The residue obtained after concentration is purified by column chromatography (silica gel, methylene chloride-methylene chloride/methanol 800:1-20:1). 90 mg (0.26 mmol, 83% yield) of the product are obtained as a colorless crystalline solid.

R_{f}: 0.60 (cyclohexane/ethyl acetate 2:1).

¹H-NMR (300 MHz, CDCl₃, δ/ppm): 8.50 (d, 1H), 7.65 (m, 2H), 7.36 (m, 2H), 7.07 (d, 1H), 7.01 (d, 1H), 3.82 (s, 3H), 2.93 (dt, 1H), 2.71 (dd, 1H), 2.62-2.36 (m, 2H), 2.06 (m, 1H), 1.70 (m, 1H), 1.48-1.36 (m, 3H), 0.99 (t, 3H).

MS (EI): 350 (M⁺).

### EXAMPLE 4-24

### 4-(3-Ethyl-9-oxo-2,3,4,9-tetrahydro-1-thioxanthen-6-yl)benzaldehyde

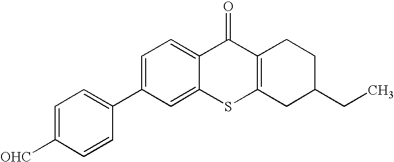

81 mg (0.23 mmol, 75% yield) of the product are obtained as a colorless crystalline solid from 100 mg (0.31 mmol) of 6-bromo-3-ethyl-1,2,3,4-tetrahydro-9H-thioxanthen-9-one and 56 mg (0.37 mmol) of 4-formylphenylboronic acid in analogy to the synthesis of 3-ethyl-6-(2-methoxyphenyl)-1,2,3,4-tetrahydro-9H-thioxanthen-9-one.

R_{f}: 0.52 (cyclohexane/ethyl acetate 2:1).

¹H-NMR (300 MHz, CDCl₃, δ/ppm): 10.10 (s, 1H), 8.60 (d, 1H), 8.00 (d, 2H), 7.82 (d, 2H), 7.75 (d, 1H), 7.72 (dd, 1H). 2.96 (dt, 1H), 2.77 (dd, 1H), 2.67-2.36 (m, 2H), 2.08 (m, 1H), 1.73 (m, 1H), 1.50-1.32 (m, 3H), 1.03 (t, 3H).

MS (ESI): 349.2 ([M+H]⁺).

### EXAMPLE 4-25

### 3-(3-Ethyl-9-oxo-2,3,4,9-tetrahydro-1-thioxanthen-6-yl)benzaldehyde

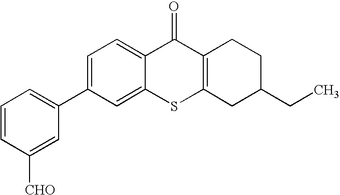

72 mg (0.21 mmol, 67% yield) of the product are obtained as a colorless crystalline solid from 100 mg (0.31 mmol) of 6-bromo-3-ethyl-1,2,3,4-tetrahydro-9H-thioxanthen-9-one and 56 mg (0.37 mmol) of 3-formylphenylboronic acid in analogy to the synthesis of 3-ethyl-6-(2-methoxyphenyl)-1,2,3,4-tetrahydro-9H-thioxanthen-9-one.

R_{f}: 0.54 (cyclohexane/ethyl acetate 2:1).

¹H-NMR (300 MHz, CDCl₃, δ/ppm): 10.12 (s, 1H), 8.59 (d, 1H), 8.16 (m, 1H), 7.92 (m, 2H), 7.75-7.63 (m, 3H), 2.96 (dt, 1H), 2.75 (dd, 1H), 2.62-2.39 (m, 2H), 2.06 (m, 1H), 1.72 (m, 1H), 1.50-1.37 (m, 3H), 1.00 (t, 3H).

MS (ESI): 349.2 ([M+H]⁺).

### EXAMPLE 4-26

### 5-(3-Ethyl-9-oxo-2,3,4,9-tetrahydro-1H-thioxanthen-6-yl)-2-thiophenecarbaldehyde

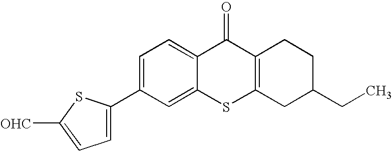

252 mg (0.71 mmol, 76% yield) of the product are obtained as a pale yellow crystalline solid from 300 mg (0.93 mmol) of 6-bromo-3-ethyl-1,2,3,4-tetrahydro-9H-thioxanthen-9-one and 173 mg (1.11 mmol) of 5-formyl-2-thienylboronic acid in analogy to the synthesis of 3-ethyl-6-(2-methoxyphenyl)-1,2,3,4-tetrahydro-9H-thioxanthen-9-one.

R_{f}: 0.38 (cyclohexane/ethyl acetate 2:1).

¹H-NMR (300 MHz, CDCl₃, δ/ppm): 9.94 (s, 1H), 8.55 (d, 1H), 7.79 (d, 1H), 7.77 (d, 1H), 7.74 (dd, 1H), 7.54 (d, 1H), 2.94 (dt, 1H), 2.74 (dd, 1H), 2.62-2.37 (m, 2H), 2.07 (m, 1H), 1.73 (m, 1H), 1.49-1.30 (m, 3H), 1.01 (t, 3H).

MS (ESI): 354.9 ([M+H]⁺).

### EXAMPLE 4-27

### 3-Ethyl-6-(4-hydroxyphenyl)-1,2,3,4-tetrahydro-9H-thioxanthen-9-one

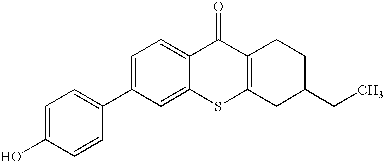

100 mg (0.31 mmol) of 3-ethyl-6-(4-methoxyphenyl)-1,2,3,4-tetrahydro-9H-thioxanthen-9-one are introduced into 20 ml of methylene chloride under an argon atmosphere. 1.43 ml of a 1M boron tribromide solution in methylene chloride are slowly added to this solution while cooling in ice, and the mixture is stirred at room temperature for 3 h. Then, while cooling in ice, 3 ml of methanol are cautiously added to the solution. The residue obtained after concentration is dissolved in methylene chloride and washed with water. Drying of the organic phase over sodium sulfate and concentration result in 90 mg (0.27 mmol, 94% yield) of the product as a pale yellow amorphous solid.

R_{f}: 0.20 (cyclohexane/ethyl acetate 2:1).

¹H-NMR (300 MHz, CDCl₃, δ/ppm): 8.52 (d, 1H), 7.68 (dd, 1H), 7.64 (d, 1H), 7.58 (d, 2H), 6.95 (d, 2H), 5.06 (s, 1H), 2.96 (dt, 1H), 2.74 (dd, 1H), 2.65-2.35 (m, 2H), 2.07 (m, 1H), 1.72 (m, 1H), 1.51-1.36 (m, 3H), 1.00 (t, 3H).

MS (EI): 336 (M⁺).

### EXAMPLE 4-28

### 3-Ethyl-6-(3-hydroxyphenyl)-1,2,3,4-tetrahydro-9H-thioxanthen-9-one

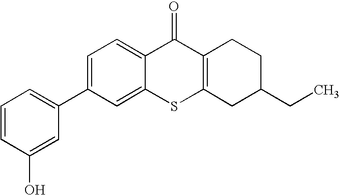

510 mg (1.46 mmol) of 3-ethyl-6-(3-methoxyphenyl)-1,2,3,4-tetrahydro-9H-thioxanthen-9-one are introduced into 40 ml of methylene chloride under an argon atmosphere. 5 ml of a 1M boron tribromide solution in methylene chloride is slowly added to this solution while cooling in ice, and the mixture is stirred at room temperature for 3 h. Then, while cooling in ice, 8 ml of methanol are cautiously added to the solution. The residue obtained after concentration is dissolved in methylene chloride and washed with water. Drying of the organic phase over sodium sulfate and concentration result in 440 mg (1.30 mmol, 90% yield) of the product as a pale yellow amorphous solid.

R_{f}: 0.24 (cyclohexane/ethyl acetate 2:1).

¹H-NMR (300 MHz, CDCl₃, δ/ppm): 8.49 (d, 1H), 7.69 (d, 1H), 7.66 (dd, 1H), 7.27 (d, 1H), 7.13 (m, 2H), 6.89 (m, 1H), 4.87 (s, 1H), 2.92 (dt, 1H), 2.76 (dd, 1H), 2.60-2.38 (m, 2H), 2.08 (m, 1H), 1.73 (m, 1H), 1.53-1.25 (m, 3H), 1.02 (t, 3H).

MS (ESI): 337.3 (M⁺).

### EXAMPLE 4-29

### 3-Ethyl-6-[4-(4-morpholinylmethyl)phenyl]-1,2,3,4-tetrahydro-9H-thioxanthen-9-one

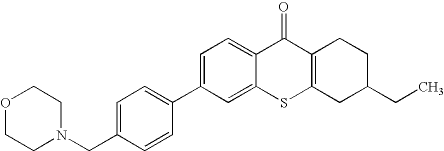

30 mg (0.09 mmol) of 4-(3-ethyl-9-oxo-2,3,4,9-tetrahydro-1H-thioxanthen-6-yl)benzaldehyde are dissolved in 5 ml of 1,2-dichloroethane under an argon atmosphere and, while stirring, 8 mg (0.09 mmol) of morpholine and 28 mg (0.13 mmol) of sodium(triacetoxyborohydride) are added. After 1 h, 5 μl of glacial acetic acid are added to this solution, and the mixture is stirred overnight. This solution is then partitioned between methylene chloride and saturated sodium bicarbonate solution, and the organic phase is washed with water and dried over sodium sulfate. Concentration results in 32 mg (0.08 mmol, 85% yield) of the product as a colorless amorphous solid.

R_{f}: 0.19 (cyclohexane/ethyl acetate 2:1).

¹H-NMR (300 MHz, CDCl₃, δ/ppm): 8.56 (d, 1H), 7.68 (m, 2H), 7.62 (d, 2H), 7.44 (d, 2H), 3.72 (t, 4H), 3.57 (s, 2H), 2.95 (dt, 1H), 2.76 (dd, 1H), 2.66-2.35 (m+t, 6H), 2.07 (m, 1H), 1.72 (m, 1H), 1.52-1.36 (m, 3H), 1.01 (t, 3H).

MS (ESI): 420.4 ([M+H]⁺).

### EXAMPLE 4-30

### N-(3-Ethyl-9-oxo-2,3,4,9-tetrahydro-1-thioxanthen-6-yl)-N-methyl-2-naphthalenesulfonamide

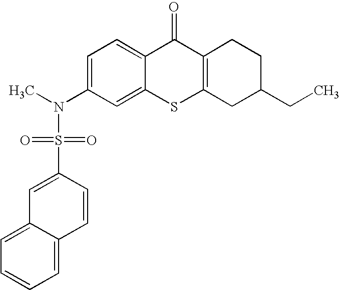

10 mg (0.02 mmol, 15% yield) of the product are obtained as a pale yellow microcrystalline solid from 58 mg (0.13 mmol) of N-(3-ethyl-9-oxo-2,3,4,9-tetrahydro-1H-thioxanthen-6-yl)-2-naphthalenesulfonamide, 75 mg (0.53 mmol) of methyl iodide and 183 mg (1.33 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.56 (cyclohexane/ethyl acetate 2:1).

MS (ESI): 464.5 ([M+H]⁺).

### EXAMPLE 4-31

### N-(3-ethyl-9-oxo-2,3,4,9-tetrahydro-1H-thioxanthen-6-yl)-N-methyl-2-thiophenesulfonamide

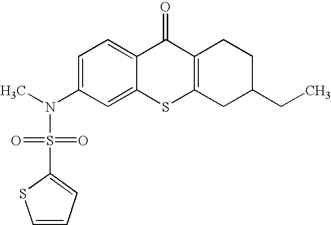

23 mg (0.05 mmol, 82% yield) of the product are obtained as a pale yellow microcrystalline solid from 28 mg (0.07 mmol) of N-(3-ethyl-9-oxo-2,3,4,9-tetrahydro-1H-thioxanthen-6-yl)-2-thiophenesulfonamide, 40 mg (0.28 mmol) of methyl iodide and 95 mg (0.69 mmol) of potassium carbonate in analogy to the synthesis of N-(3-ethyl-9-oxo-2,3,4,9-tetrahydro-1-thioxanthen-6-yl)-N-methyl-1-butanesulfonamide.

R_{f}: 0.53 (cyclohexane/ethyl acetate 2:1).

MS (ESI): 420.4 ([M+H]⁺).

### EXAMPLE 4-32

### 2-(1,3-Dioxo-1,3-dihydro-2H-isoindol-2-yl)-N-(3-ethyl-9-oxo-2,3,4,9-tetrahydro-1H-thioxanthen-6-yl)-N-methylsulfonamide

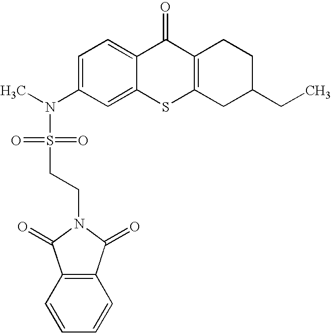

15 mg (0.03 mmol, 35% yield) of the product are obtained as a pale yellow solid from 39 mg (0.08 mmol) of 2-(1,3-dioxo-1,3-dihydro-2H-isoindol-2-yl)-N-(3-ethyl-9-oxo-2,3,4,9-tetrahydro-1H-thioxanthen-6-yl)ethanesulfonamide, 48 mg (0.34 mmol) of methyl iodide and 117 mg (0.85 mmol) of potassium carbonate in analogy to the synthesis of N-(3-ethyl-9-oxo-2,3,4,9-tetrahydro-1H-thioxanthen-6-yl)-N-methyl-1-butanesulfonamide.

R_{f}: 0.41 (cyclohexane/ethyl acetate 2:1).

MS (ESI): 511.2 ([M+H]⁺).

### EXAMPLE 4-33

### 3,3-Dimethyl-6-(4-methyl-3-pyridinyl)-1,2,3,4-tetrahydro-9H-thioxanthen-9-one

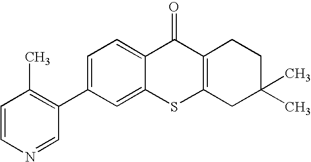

49 mg (0.15 mmol, 31% yield) of the product are obtained as a colorless crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 103 mg (0.60 mmol) of 3-bromo-4-methylpyridine in analogy to the synthesis of 3-ethyl-6-(2-pyridinyl)-1,2,3,4-tetrahydro-9H-thioxanthen-9-one.

R_{f}: 0.48 (cyclohexane/ethyl acetate 2:1).

MS (ESD: 336 ([M+H]⁺).

### EXAMPLE 4-34

### 6-(6-Methoxy-3-pyridinyl)-3,3-dimethyl-1,2,3,4-tetrahydro-9H-thioxanthen-9-one

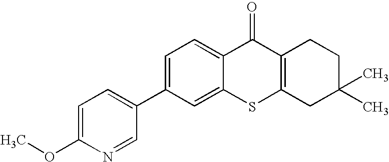

68 mg (0.19 mmol, 42% yield) of the product are obtained as a pale yellow crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 113 mg (0.60 mmol) of 3-bromo-6-methoxypyridine in analogy to the synthesis of 3-ethyl-6-(2-pyridinyl)-1,2,3,4-tetrahydro-9H-thioxanthen-9-one.

R_{f}: 0.42 (cyclohexane/ethyl acetate 2:1).

¹H-NMR (300 MHz, CDCl₃, δ/ppm): 8.58 (d, 1H), 8.45 (d, 1H), 7.84 (dd, 1H), 7.63 (m, 2H), 6.86 (d, 1H), 4.01 (s, 3H), 2.73 (t, 2H), 2.49 (s, 2H), 1.64 (t, 2H) 1.06 (s, 6H).

MS (ESI): 352 ([M+H]⁺).

### EXAMPLE 4-35

### 6-(4-Methoxy-5-pyrimidinyl)-3,3-dimethyl-1,2,3,4-tetrahydro-9H-thioxanthen-9-one

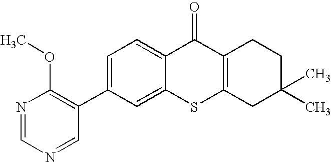

62 mg (0.18 mmol, 43% yield) of the product are obtained as a pale yellow crystalline solid from 132 mg (0.41 mmol) of 6-bromo-3,3-dimethyl-1,2,3,4-tetrahydro-9H-thioxanthen-9-one, 124 mg (0.49 mmol) of bis(pinacolato)diboron, 119 mg (1.22 mmol) of potassium acetate and 100 mg (0.53 mmol) of 3-bromo-4-methoxypyrimidine in analogy to the synthesis of 3-ethyl-6-(2-pyridinyl)-1,2,3,4-tetrahydro-9H-thioxanthen-9-one.

R_{f}: 0.22 (cyclohexane/ethyl acetate 2:1).

¹H-NMR (300 MHz, CDCl₃, δ/ppm): 8.80 (s, 1H), 8.58 (d, 1H), 8.53 (s, 1H), 7.69 (d, 1H), 7.63 (dd, 1H), 4.07 (s, 3H), 2.73 (t, 2H), 2.49 (s, 2H), 1.63 (t, 2H), 1.04 (s, 6H).

MS (ESI): 353 ([M+H]⁺).

### EXAMPLE 4-36

### 3,3-Dimethyl-6-(5-methyl-3-pyridinyl)-1,2,3,4-tetrahydro-9H-thioxanthen-9-one

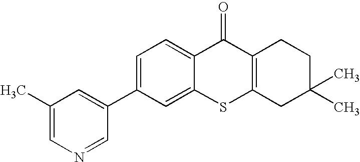

37 mg (0.11 mmol, 24% yield) of the product are obtained as a colorless crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 102 mg (0.60 mmol) of 3-bromo-5-methylpyridine in analogy to the synthesis of 3-ethyl-6-(2-pyridinyl)-1,2,3,4-tetrahydro-9H-thioxanthen-9-one.

R_{f}: 0.39 (cyclohexane/ethyl acetate 2:1).

MS (ESI): 336 ([M+H]⁺).

### EXAMPLE 4-37

### 6-(5-Acetyl-3-pyridinyl)-3,3-dimethyl-1,2,3,4-tettahydro-9H-thioxanthen-9-one

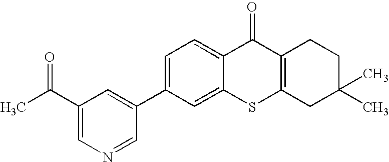

34 mg (0.09 mmol, 20% yield) of the product are obtained as a colorless crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 120 mg (0.60 mmol) of 3-bromo-5-acetylpyridine in analogy to the synthesis of 3-ethyl-6-(2-pyridinyl)-1,2,3,4-tetrahydro-9H-thioxanthen-9-one.

R_{f}: 0.42 (cyclohexane/ethyl acetate 2:1).

¹H-NMR (300 MHz, CDCl₃, δ/ppm): 9.19 (d, 1H), 9.05 (d, 1H), 8.62 (d, 1H), 8.47 (t, 1H), 7.71 (m, 2H), 2.77 (t, 2H), 2.72 (s, 3H), 2.51 (s, 2H), 1.65 (t, 2H), 1.06 (s, 6H).

MS (ESI): 364 ([M+H]⁺).

### EXAMPLE 4-38

### Methyl 5-(3,3-dimethyl-9-oxo-2,3,4,9-tetrahydro-1-thioxanthen-6-yl)nicotinate

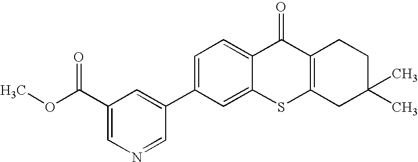

104 mg (0.27 mmol, 59% yield) of the product are obtained as a colorless crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 130 mg (0.60 mmol) of methyl 3-bromonicotinate in analogy to the synthesis of 3-ethyl-6-(2-pyridinyl)-1,2,3,4-tetrahydro-9H-thioxanthen-9-one.

R_{f}: 0.40 (cyclohexane/ethyl acetate 2:1).

¹H-NMR (300 MHz, CDCl₃, δ/ppm): 9.26 (d, 1H), 9.05 (d, 1H), 8.62 (d, 1H), 8.58 (t, 1H), 7.72 (m, 2H), 4.01 (s, 3H), 2.75 (t, 2H), 2.51 (s, 2H), 1.66 (t, 2H), 1.05 (s, 6H).

MS (ESI): 380 ([M+H]⁺).

### EXAMPLE 4-39

### 6-(5,6-Dimethyl-3-pyridinyl)-3,3-dimethyl-1,2,3,4-tetrahydro-9H-thioxanthen-9-one

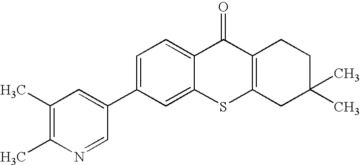

61 mg (0.17 mmol, 38% yield) of the product areobtained as a colorless crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 112 mg (0.60 mmol) of 3-bromo-5,6-dimethylpyridine in analogy to the synthesis of 3-ethyl-6-(2-pyridinyl)-1,2,3,4-tetrahydro-9H-thioxanthen-9-one.

R_{f}: 0.37 (cyclohexane/ethyl acetate 2:1).

MS (ESI): 349 ([M]⁺).

### EXAMPLE 4-40

### 6-(4,6-Dimethoxy-5-pyrimidinyl)-3,3-dimethyl-1,2,3,4-tetrahydro-9H-thioxanthen-9-one

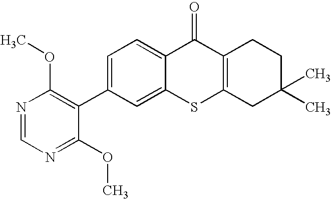

104 mg (0.27 mmol, 59% yield) of the product are obtained as a colorless crystalline solid from 150 mg (0.46 mmol) of 6-bromo-3,3-dimethyl-1,2,3,4-tetrahydro-9H-thioxanthen-9-one, 142 mg (0.56 mmol) of bis(pinacolato)diboron, 136 mg (1.39 mmol) of potassium acetate and 131 mg (0.60 mmol) of 3-bromo-4,6-dimethoxypyrimidine in analogy to the synthesis of 3-ethyl-6-(2-pyridinyl)-1,2,3,4-tetrahydro-9H-thioxanthen-9-one.

R_{f}: 0.25 (cyclohexane/ethyl acetate 2:1).

¹H-NMR (300 MHz, CDCl₃, δ/ppm): 8.52 (d, 1H), 8.46 (s, 1H), 7.56 (d, 1H), 7.50 (dd, 1H), 3.95 (s, 6H), 2.73 (t, 2H), 2.48 (s, 2H), 1.63 (t, 2H), 1.03 (s, 6H).

MS (ESI): 383 ([M+H]⁺).

### EXAMPLE 4-41

### 3-(3-Ethyl-9-oxo-2,3,4,9-tetrahydro-1-thioxanthen-6-yl)-2-thiophenecarbaldehyde

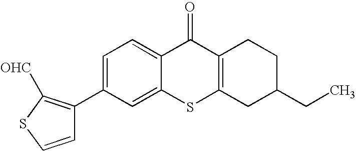

210 mg (0.59 mmol, 63% yield) of the product are obtained as a pale yellow crystalline solid from 300 mg (0.93 mmol) of 6-bromo-3-ethyl-1,2,3,4-tetrahydro-9H-thioxanthen-9-one and 173 mg (1.11 mmol) of 2-formylthiophene-3-boronic acid in analogy to the synthesis of 3-ethyl-6-(2-methoxyphenyl)-1,2,3,4-tetrahydro-9H-thioxanthen-9-one.

R_{f}: 0.41 (cyclohexane/ethyl acetate 2:1).

MS (ESI): 355.2 ([M+H]⁺).

### EXAMPLE 4-42

### 3,3-Dimethyl-6-(5-pyrinmidinyl)-1,2,3,4-tetrahydro-9H-thioxanthen-9-one

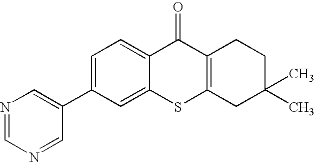

0.9 g (2.79 mmol, 90% yield) of the product is obtained as a colorless crystalline solid from 1.00 g (3.09 mmol) of 6-bromo-3,3-dimethyl-1,2,3,4-tetrahydro-9H-thioxanthen-9-one and 1.02 g (4.95 mmol) of 5-(4,4,5,5-tetramethyl-1,3,2-dioxaborolan-2-yl)pyrimidine with additional use of potassium phosphate as base and dichloro[bis(diphenylphosphino)ferrocenyl]palladium(II) as catalyst in analogy to the synthesis of 3-ethyl-6-(2-methoxyphenyl)-1,2,3,4-tetrahydro-9H-thioxanthen-9-one.

R_{f}: 0.18 (cyclohexane/ethyl acetate 2:1).

¹H-NMR (300 MHz, CDCl₃, δ/ppm): 9.29 (s, 1H), 9.02 (s, 2H), 8.65 (d, 1H), 7.69 (m, 2H), 2.75 (t, 2H), 2.51 (s, 2H), 1.66 (t, 2H), 1.05 (s, 6H).

MS (ESI): 323 ([M+H]⁺).

### EXAMPLE 4-43

### 6-(4-Methoxy-3-pyridinyl)-3,3-dimethyl-1,2,3,4-tetrahydro-9H-thioxanthen-9-one

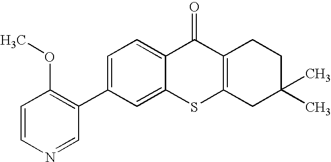

60 mg (0.17 mmol, 22% yield) of the product are obtained as a colorless solid from 240 mg (0.75 mmol) of 6-bromo-3,3-dimethyl-1,2,3,4-tetrahydro-9H-thioxanthen-9-one and 150 mg (0.98 mmol) of 4-methoxy-3-pyridineboronic acid in analogy to the synthesis of 3-ethyl-6-(2-methoxyphenyl)-1,2,3,4-tetrahydro-9H-thioxanthen-9-one.

R_{f}: 0.38 (cyclohexane/ethyl acetate 2:1).

MS (ESI): 351 ([M]⁺).

### EXAMPLE 4-44

### 3-Ethyl-6-[5-(4-morpholinylmethyl)-2-thienyl]-1,2,3,4-tetrahydro-9H-thioxanthen-9-one

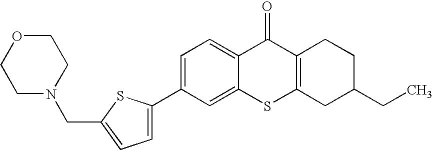

13 mg (0.03 mmol, 15% yield) of the product are obtained as a colorless solid from 71 mg (0.20 mmol) of 5-(3-ethyl-9-oxo-2,3,4,9-tetrahydro-1H-thioxanthen-6-yl)-2-thiophenecarbaldehyde, 19 mg (0.22 mmol) of morpholine, 63 mg (0.30 mmol) of sodium(triacetoxyborohydride) and 20 μl of glacial acetic acid in analogy to the synthesis of 3-ethyl-6-[4-(4-morpholinylmethyl)phenyl]-1,2,3,4-tetrahydro-9H-thioxanthen-9-one.

R_{f}: 0.21 (cyclohexane/ethyl acetate 2:1).

MS (ESI): 425 ([M]⁺).

1. A compound of the formula (I), 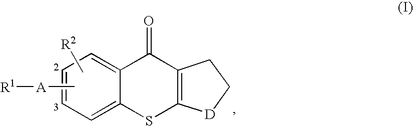
in which
the radical R¹-A- is located at either of positions 2 or 3 of the thiochromenone ring,
R¹ is (C₆-C₁₀)-aryl or 5- to 10-membered heteroaryl, where aryl and heteroaryl are optionally substituted identically or differently by radicals selected from the group of halogen, formyl, carbamoyl, cyano, hydroxyl, trifluoromethoxy, nitro, —NR³R⁴, tetrazolyl, (C₁-C₆)-alkoxycarbonyl and optionally hydroxyl-, morpholinyl-, (C₁-C₆)-acyloxy- or halogen-substituted (C₁-C₆)-alkyl, (C₁-C₆)-alkoxy, (C₁-C₆)-acyl and (C₁-C₆)-alkylthio,
in which R³ and R⁴ are, independently of one another, hydrogen, (C₁-C₆)-alkyl or (C₁-C₆)-acyl, is 3- to 12-membered carbocyclyl or 4- to 12-membered heterocyclyl, where carbocyclyl and heterocyclyl are optionally substituted identically or differently by radicals selected from the group of (C₁-C₆)-alkyl, (C₁-C₆)-alkoxy, (C₁-C₆)-acyl, (C₁-C₆)-alkoxycarbonyl or oxo,
or
is a group of the formula R⁵-E-,
in which
E is optionally unsaturated (C₁-C₁₀)-alkanediyl, and
R⁵ is hydrogen, carbamoyl, halogen, hydroxyl, nitro, trifluoromethyl, amino, mono-(C₁-C₆)-alkylamino, di-(C₁-C₆)-alkylamino, (C₁-C₆)-alkoxy, (C₆-C₁₀)-aryl, 5- to 10-membered heteroaryl or 4- to 10-membered, optionally oxo- and/or (C₁-C₆)-alkyl-substituted, optionally benzo-fused heterocyclyl, where aryl, heteroaryl and benzo in turn may be substituted by radicals selected from the group of halogen, cyano, trifluoromethyl, trifluoromethoxy, nitro and (C₁-C₆)-alkyl,
A is a bond or a group of the formula O, S, NR⁶, CO, SO, SO₂, SO₂—O, CO—NR⁷, SO₂—NR⁸, O—SO₂, NR⁹—CO, NR¹⁰—SO₂, NR¹¹—SO₂—O, NR¹²—SO₂—NR¹³ or NR¹⁴—CO—NR¹⁵,
in which R⁶ R⁷, R⁸, R⁹, R¹⁰, R¹¹, R¹², R¹³, R¹⁴ and R¹⁵ are (C₃-C₈)-cycloalkyl or optionally unsaturated (C₁-C₆)-alkyl which is optionally substituted by hydroxyl, phenyl, (C₁-C₆)-alkoxy, (C₁-C₆)-alkoxycarbonyl or (C₃-C₈)-cycloalkyl, where phenyl in turn may be substituted by halogen or (C₁-C₄)-alkyl, or
in which R⁶, R⁷, R⁹, R¹⁰, R¹¹, R¹², R¹⁴ and R¹⁵ are hydrogen,
or
the radical R¹-A- is hydrogen or amino,
R² is hydrogen, halogen or (C₁-C₆)-alkyl or (C₁-C₆)-alkoxy, where alkyl and alkoxy are optionally substituted up to twice-identically or differently by radicals selected from the group of hydroxyl, (C₁-C₆)-alkoxy, mono- and di-(C₁-C₆)-alkylamino, and
D is an optionally fluorine-substituted, divalent hydrocarbon radical having 3 to 10 carbon atoms,
and the salts, hydrates and/or solvates thereof,
with the exception of 2-chloro-6,7,8,9,10,10a-hexahydrocyclohepta[b]thio-chromen-11(5aH)-one.

2. A compound of the formula (I) as claimed in claim 1, where
the radical R¹-A- is located at position 3 of the thiochromenone ring,
R¹ is (C₆-C₁₀)-aryl or 5- to 10-membered heteroaryl, where aryl and heteroaryl are optionally substituted identically or differently up to twice by radicals selected from the group of halogen, formyl, cyano, hydroxyl, hydroxymethyl, (C₁-C₆)-alkyl, is 4- to 10-membered heterocyclyl, where heterocyclyl are optionally substituted identically or differently by radicals selected from the group of (C₁-C₆)-alkyl, (C₁-C₆)-alkoxy, (C₁-C₆)-alkoxycarbonyl or oxo,
A is a bond or a group of the formula NR⁶, CO—NR⁷, SO₂—NR⁸ or NR⁹—CO,
in which R⁶, R⁷, R⁸ and R⁹ are optionally unsaturated (C₁-C₆)-alkyl which is optionally substituted up to twice, identically or differently, by hydroxyl or methoxy, or
in which R⁶, R⁷ and R⁹ are hydrogen,
R² is hydrogen, and
D is a group of the formula (CH₂)ₘ—CR¹⁶R¹⁷—(CH₂)ₙ,
in which
the total number of carbon atoms is 3 to 10,
m and n are identical or different and are a natural number from the series 0 to 6,
and
R¹⁶ and R¹⁷ are identical or different and are hydrogen or (C₁-C₆)-alkyl which is optionally substituted identically or differently by (C₃-C₅)-cycloalkyl or halogen,
or
CR¹⁶R¹⁷ is (C₃-C₆)-cycloalkane-1,1-diyl,
and the salts, hydrates and/or solvates thereof.

3. A compound of the formula (I) as claimed in claim 1, where
the radical R¹-A- is located at position 3 of the thiochromenone ring,
R¹ is phenyl or 5- to 6-membered heteroaryl, where phenyl and heteroaryl are optionally substituted identically or differently up to twice by radicals selected from the group of halogen, cyano, (C₁-C₃)-alkyl, is 5- to 7-membered heterocyclyl, where heterocyclyl are optionally substituted identically or differently by radicals selected from the group of (C₁-C₃)-alkyl or Oxo,
A is a bond or a group of the formula NR⁶, SO₂—NR⁸ or NR⁹—CO,
in which R⁶, R⁸ and R⁹ are optionally unsaturated (C₁-C₃)-alkyl which is optionally substituted up to twice, identically or differently, by hydroxyl or methoxy, and in which R⁶, R⁸ and R⁹ are hydrogen,
R² is hydrogen, and
D is a group of the formula (CH₂)ₘ—CR¹⁶R¹⁷—(CH₂)ₙ,
in which
the total number of carbon atoms is 3 to 6,
m and n are identical or different and are a natural number from the series 0 to 2,
and
R¹⁶ and R¹⁷ are identical or different and are hydrogen or (C₁-C₃)-alkyl,
or
CR¹⁶R¹⁷ is (C₃-C₆)-cycloalkane-1,1-diyl,
and the salts, hydrates and/or solvates thereof.

4. A compound of the formula (I) as claimed in claim 1 for the treatment and/or prophylaxis of diseases.

5. A medicament comprising at least one of the compounds of the formula (I) as claimed in any of claims 1 to 3 mixed with at least one pharmaceutically acceptable, essentially non-toxic carrier or excipient.

6. The use of compounds of the formula (I) as claimed in any of claims 1 to 3 for producing a medicament for the treatment and/or prophylaxis of states of pain and/or neurodegeinerative disorders.
